SEQUENCE LISTING (11 sequences)

SEQ ID NO: 1 - protein, 5 aa, Artificial Sequence
  Synthetic peptide
YIGSR

SEQ ID NO: 2 - protein, 10 aa, Artificial Sequence
  Synthetic peptide
RNIAEIIKDI

SEQ ID NO: 3 - protein, 11 aa, Artificial Sequence
  Synthetic peptide
VGKKKKKKKKG

SEQ ID NO: 4 - protein, 15 aa, Artificial Sequence
  Synthetic peptide
  MISC_FEATURE: The GAGVP are each repeated, and the entire peptide is repeated 12 times.
GAGVPGYGVPGAGVP

SEQ ID NO: 5 - protein, 15 aa, Artificial Sequence
  Synthetic peptide
  MISC_FEATURE: The GAGVP are each repeated, and the entire peptide is repeated 24 times
GAGVPGYGVPGAGVP

SEQ ID NO: 6 - protein, 16 aa, Artificial Sequence
  Synthetic peptide
YIGSRVGKKKKKKKKG

SEQ ID NO: 7 - protein, 15 aa, Artificial Sequence
  Synthentic peptide
  MISC_FEATURE: X are any amino acid except proline
  misc_feature (1)..(2): Xaa can be any naturally occurring amino acid
  misc_feature (4)..(4): Xaa can be any naturally occurring amino acid
  misc_feature (6)..(7): Xaa can be any naturally occurring amino acid
  misc_feature (9)..(9): Xaa can be any naturally occurring amino acid
  misc_feature (11)..(12): Xaa can be any naturally occurring amino acid
  misc_feature (14)..(14): Xaa can be any naturally occurring amino acid
XXGXPXXGXPXXGXP

SEQ ID NO: 8 - protein, 5 aa, Artificial Sequence
  Synthetic peptide
  MISC_FEATURE: X are any amino acid except proline
  MISC_FEATURE: X are any amino acid except proline
  misc_feature (1)..(2): Xaa can be any naturally occurring amino acid
  misc_feature (4)..(4): Xaa can be any naturally occurring amino acid
XXGXP

SEQ ID NO: 9 - protein, 10 aa, Artificial Sequence
  Synthetic peptide
  MISC_FEATURE: X are any amino acid except proline
  misc_feature (1)..(2): Xaa can be any naturally occurring amino acid
  misc_feature (4)..(4): Xaa can be any naturally occurring amino acid
  misc_feature (6)..(7): Xaa can be any naturally occurring amino acid
  misc_feature (9)..(9): Xaa can be any naturally occurring amino acid
XXGXPXXGXP

SEQ ID NO: 10 - protein, 17 aa, Artificial Sequence
  Synthetic peptide
  MISC_FEATURE: X are any amino acid except proline
  misc_feature (1)..(2): Xaa can be any naturally occurring amino acid
  misc_feature (4)..(4): Xaa can be any naturally occurring amino acid
  misc_feature (7)..(8): Xaa can be any naturally occurring amino acid
  misc_feature (10)..(10): Xaa can be any naturally occurring amino acid
  misc_feature (13)..(14): Xaa can be any naturally occurring amino acid
  misc_feature (16)..(16): Xaa can be any naturally occurring amino acid
XXGXPNXXGXPNXXGXP

SEQ ID NO: 11 - protein, 17 aa, Artificial Sequence
  Synthetic peptide
  MISC_FEATURE: X are any amino acids except proline
  misc_feature (1)..(2): Xaa can be any naturally occurring amino acid
  misc_feature (4)..(4): Xaa can be any naturally occurring amino acid
  misc_feature (7)..(8): Xaa can be any naturally occurring amino acid
  misc_feature (10)..(10): Xaa can be any naturally occurring amino acid
  misc_feature (13)..(14): Xaa can be any naturally occurring amino acid
  misc_feature (16)..(16): Xaa can be any naturally occurring amino acid
XXGXPNXXGXPNXXGXP

(19) United States
(12) Patent Application Publication
(10) Pub. No.: US 2021/0230552 A1
(43) Pub. Date: Jul. 29, 2021

(54) Biomaterials for 3D Cell Growth and Differentiation

(71) Applicant: Texas Tech University System, Lubbock, TX (US)

(72) Inventors: Harvinder Singh Gill, Lubbock, TX (US); Chang Hyun Lee, Lubbock, TX (US)

(21) Appl. No.: 17/231,495

(22) Filed: Apr. 15, 2021

Related U.S. Application Data
(63) Continuation-in-part of application No. PCT/US2019/056580, filed on Oct. 16, 2019.
(60) Provisional application No. 62/746,064, filed on Oct. 16, 2018.

(51) Int. Cl. C12N 5/077; C12N 5/00; C07K 7/06; C07K 14/52; C07K 14/475

(52) U.S. Cl. CPC C12N 5/0657; C12N 5/0062; C07K 7/06; C12N 2513/00; C07K 14/475; C12N 2501/998; C07K 2319/00; C07K 14/52

(57) ABSTRACT

The present invention includes a polypeptide for use in a three dimensional (3D) culture system for the growth of cells comprising one or more repeats of a sequence n₁-(X₁X₂GXP)-n₂ (SEQ ID NO:8), wherein X₁ and X₂ are any amino acids except proline, wherein X₁ and X2 can be the same or different amino acid in solution or coated on a substrate, wherein n₁ and n₂ are equal to or greater than one, and wherein X is an aliphatic amino acid.

### CROSS-REFERENCE TO RELATED APPLICATIONS

This application claims priority to and is a continuation-in-part application of National Stage of International Application No. of PCT/US2019/056580, filed on Oct. 16, 2019, and U.S. Provisional Application No. 62/746,064, filed on Oct. 16, 2018, the content of each of which is incorporated by reference herein.

### TECHNICAL FIELD OF THE INVENTION

The present invention relates in general to the field of biomaterials for cell culture, and more particularly, to novel biomaterials for 3D cell growth and differentiation.

### STATEMENT OF FEDERALLY FUNDED RESEARCH

None.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on Mar. 9, 2021, is named TECH2133WO_SeqLst.txt and is 6 kilobytes in size.

### BACKGROUND OF THE INVENTION

Without limiting the scope of the invention, its background is described in connection with systems and biomaterials for cell culture.

One such system is taught in U.S. Pat. No. 9,694,107, issued to Nakamura, et al., entitled “Scaffold-free self-organized 3D synthetic tissue”. These inventors teach a synthetic tissue or complex produced by culture that is said to have a high level of differentiation ability. These inventors culture cells under specific culture conditions such that medium contains an extracellular matrix synthesis promoting agent, the cells are organized and are easily detached from a culture dish. These inventors are also said to teach a method for producing an implantable synthetic tissue that does not require a plurality of monolayer cell sheets assembled to form a three-dimensionally structured synthetic tissue.

Another such system is taught in U.S. Pat. No. 9,604,407, issued to Leighton, et al., and entitled, “3D printing techniques for creating tissue engineering scaffolds”. Briefly, these inventors are said to teach a three-dimensional tissue scaffold in which a first layer of scaffold fiber is printed with a printer onto a base gel substrate and disposing a first gel layer over the printed first layer. In an alternative embodiment, these inventors are said to teach printing a first and second sacrificial fiber with a printer onto a base gel substrate, printing a first scaffold fiber between the first and second sacrificial fiber to form a printed first layer, and disposing a first gel layer over the printed first layer.

Another such system is taught in U.S. Pat. No. 7,452,718, issued to Gold, et al., and entitled “Direct differentiation method for making cardiomyocytes from human embryonic stem cells”. Briefly, these inventors are said to teach a procedure for generating cells of cardiomyocyte lineage from embryonic stem cells for use in regenerative medicine by differentiating by way of embryoid body formation in which serum is no longer required. Instead, these inventors are said to teach plating stem cells on a solid substrate, and differentiated the stem cells in the presence of select factors and morphogens.

However, a need remains for improved matrices for growing and differentiating cells in tissue culture that provides a high yield and in which the cells more closely resemble cells in tissues.

### SUMMARY OF THE INVENTION

In one embodiment, the present invention includes a polypeptide for use in a three dimensional (3D) culture system for the growth of cells comprising: one or more repeats of a sequence n₁-(X₁X₂GXP)-n₂ (SEQ ID NO:8), wherein X, X₁, X₂ are any amino acid, wherein X, X₁, and X₂ can be the same or different amino acid, wherein n₁ and n₂ are equal to or greater than one. In one aspect, the polypeptide has the sequence selected from at least one of [(X₁X₂GXP)(X₃X₄GXP)]ₙ₁, [(X₁X₂GXP)ₙ₁(X₃X₄GXP)ₙ₂], or [(X₁X₂GXP)ₙ₁(X₃X₄GXP)ₙ₂(X₁X₂GXP)ₙ₁], wherein X, X₁, X₂, X₃, and X₄ are any amino acid, and n₁ and n₂ are greater than or equal to one; wherein X₁ and X₂ can be the same or different from each other, and X₃ and X₄ can be the same or different from each other; or wherein at least one of X₁ or X₂ is different from X₃ or X₄, or wherein X is valine, or X₁=G, X₂=Y and A (in 1:4 ratio) and X=V. In another aspect, the polypeptide further comprises attaching to, or forming a fusion protein with, the polypeptide and at least a portion of an extracellular matrix component selected from at least one of: glycosaminoglycans (GAGs), proteoglycans, and/or proteins such as but not limited to laminin, fibronectin, vitronectin, collagen, elastin, fibrillin, fibulin, tenascin, perlecan, versican, aggrecan, neurocan, brevican, keratan, hyaluronic acid, heparan, or chondroitin, and wherein the fusion protein can be at an amino, a carboxy, or both the amino and carboxy ends of the polypeptide. In another aspect, the polypeptide further comprises attaching to, or forming a fusion protein with, the polypeptide and at least a portion of a growth factor or cytokine selected from at least one of: leukemia inhibitory factor, insulin, insulin like growth factors, epidermal growth factor, fibroblast growth factors including basic fibroblast growth factor, vascular endothelial growth factor, transforming growth factor-β, platelet-derived growth factor, neurotrophic factors, interleukin-2, stem cell factor, Fms-like tyrosine kinase 3/fetal liver kinase-2, granulocyte-macrophage colony-stimulating factor, interleukin 1 alpha, or granulocyte colony-stimulating factor, and wherein the fusion protein can be at an amino, a carboxy, or both the amino and carboxy ends of the polypeptide. In another aspect, the polypeptide further comprises attaching to, or forming a fusion protein with, the polypeptide and at least a portion of a growth factor/cytokine and an extracellular matrix component, wherein the fusion proteins can be at an amino, a carboxy, or both the amino and carboxy ends of the polypeptide. In another aspect, the polypeptide is provided in solution, attached to a substrate, or both. In another aspect, the polypeptide is a fusion protein with an amino-terminal end that comprises a laminin domain and a carboxy-terminal end comprises an elastin domain. In another aspect, the polypeptide comprises at least one of: (1) a laminin domain comprising one or more VGKKKKKKKKG motifs (SEQ ID NO: 3); (2) one or more YIGSRVGKKKKKKKKG motifs (SEQ ID NO: 6); (3) one or more RNAIAEIIKDI motifs (SEQ ID NO: 2); (4) an elastin domain comprising one or more [(GAGVP)₂(GYGVP)(GAGVP)₂]₁₂ motifs (SEQ ID NO: 4); (5) [(GAGVP)₂(GYGVP)(GAGVP)₂]₂₄ motifs (SEQ ID NO: 5); (6) SEQ ID NOS: 1 and 4, (7) SEQ ID NOS: 1 and 5; (8) SEQ ID NOS: 3 and 4; (9) SEQ ID NOS: 3 and 5; (10) SEQ ID NOS: 2 and 4, or (6) SEQ ID NOS: 2 and 5; (11) any combination of SEQ ID NOS: 1, 2, 3, 4, or 5, wherein the polypeptide has the sequence selected from at least one of [(X₁X₂GXP)(X₃X₄GXP)]ₙ₁, [(X₁X₂GXP)ₙ₁(X₃X₄GXP)₂]; (12) [(X₁X₂GXP)ₙ₁(X₃X₄GXP)ₙ₂(X₁X₂GXP)ₙ₁], wherein X₁, X₂, X₃, X₄, and X are any amino acid except proline; (13) [(X₁X₂GXP)(X₃X₄GXP)]ₙ₁, [(X₁X₂GXP)ₙ₁(X₃X₄GXP)ₙ₂]; or (14) [(X₁X₂GXP)ₙ₁(X₃X₄GXP)ₙ₂(X₁X₂GXP)ₙ₁], wherein X₁, X₂, X₃, X₄ is any amino acid and X is an aliphatic amino acid.

In one embodiment, the present invention includes a nucleic acid that encodes a polypeptide that comprises one or more repeats of a sequence n₁-(X₁X₂GXP)-n₂ (SEQ ID NO:8), wherein X, X₁, X₂ are any amino acid, wherein X, X₁, and X₂ can be the same or different amino acid, wherein n₁ and n₂ are equal to or greater than one. In one aspect, the polypeptide has the sequence selected from at least one of [(X₁X₂GXP)(X₃X₄GXP)]ₙ₁, [(X₁X₂GXP)ₙ₁(X₃X₄GXP)ₙ₂], or [(X₁X₂GXP)ₙ₁(X₃X₄GXP)ₙ₂(X₁X₂GXP)ₙ₁], wherein X, X₁, X₂, X₃, and X₄ are any amino acid, and n₁ and n₂ are greater than or equal to one; wherein X₁ and X₂ can be the same or different from each other, and X₃ and X₄ can be the same or different from each other; or wherein at least one of X₁ or X₂ is different from X₃ or X₄, or wherein X is valine, or X₁32 G, X₂=Y and A (in 1:4 ratio) and X=V. In another aspect, the polypeptide further comprises attaching to, or forming a fusion protein with, the polypeptide and at least a portion of an extracellular matrix component selected from at least one of: glycosaminoglycans (GAGs), proteoglycans, and/or proteins such as but not limited to laminin, fibronectin, vitronectin, collagen, elastin, fibrillin, fibulin, tenascin, perlecan, versican, aggrecan, neurocan, brevican, keratan, hyaluronic acid, heparan, or chondroitin, and wherein the fusion protein can be at an amino, a carboxy, or both the amino and carboxy ends of the polypeptide. In another aspect, the polypeptide further comprises attaching to, or forming a fusion protein with, the polypeptide and at least a portion of a growth factor or cytokine selected from at least one of: leukemia inhibitory factor, insulin, insulin like growth factors, epidermal growth factor, fibroblast growth factors including basic fibroblast growth factor, vascular endothelial growth factor, transforming growth factor-β, platelet-derived growth factor, neurotrophic factors, interleukin-2, stem cell factor, Fms-like tyrosine kinase 3/fetal liver kinase-2, granulocyte-macrophage colony-stimulating factor, interleukin 1 alpha, or granulocyte colony-stimulating factor, and wherein the fusion protein can be at an amino, a carboxy, or both the amino and carboxy ends of the polypeptide. In another aspect, the polypeptide further comprises attaching to, or forming a fusion protein with, the polypeptide and at least a portion of a growth factor/cytokine and an extracellular matrix component, wherein the fusion proteins can be at an amino, a carboxy, or both the amino and carboxy ends of the polypeptide. In another aspect, the polypeptide is provided in solution, attached to a substrate, or both. In another aspect, the polypeptide is a fusion protein with an amino-terminal end that comprises a laminin domain and a carboxy-terminal end comprises an elastin domain. In another aspect, the polypeptide comprises at least one of: (1) a laminin domain comprising one or more VGKKKKKKKKG motifs (SEQ ID NO: 3); (2) one or more YIGSRVGKKKKKKKKG motifs (SEQ ID NO: 6); (3) one or more RNAIAEIIKDI motifs (SEQ ID NO: 2); (4) an elastin domain comprising one or more [(GAGVP)₂(GYGVP)(GAGVP)₂]₁₂ motifs (SEQ ID NO: 4); (5) [(GAGVP)₂(GYGVP)(GAGVP)₂]₂₄ motifs (SEQ ID NO: 5); (6) SEQ ID NOS: 1 and 4, (7) SEQ ID NOS: 1 and 5; (8) SEQ ID NOS: 3 and 4; (9) SEQ ID NOS: 3 and 5; (10) SEQ ID NOS: 2 and 4, or (6) SEQ ID NOS: 2 and 5; (11) any combination of SEQ ID NOS: 1, 2, 3, 4, or 5, wherein the polypeptide has the sequence selected from at least one of [(X₁X₂GXP)(X₃X₄GXP)]ₙ₁, [(X₁X₂GXP)ₙ₁(X₃X₄GXP)ₙ₂]; (12) [(X₁X₂GXP)ₙ₁(X₃X₄GXP)ₙ₂(X₁X₂GXP)ₙ₁], wherein X₁, X₂, X₃, X₄, and X are any amino acid except proline; (13) [(X₁X₂GXP)(X₃X₄GXP)]ₙ₁, [(X₁X₂GXP)ₙ₁(X₃X₄GXP)ₙ₂]; or (14) [(X₁X₂GXP)ₙ₁(X₃X₄GXP)ₙ₂(X₁X₂GXP)ₙ₁], wherein X₁, X₂, X₃, X₄ is any amino acid and X is an aliphatic amino acid.

In one embodiment, the present invention includes a nucleic acid vector that encodes a polypeptide that comprises one or more repeats of a sequence n₁-(X₁X₂GXP)-n₂ (SEQ ID NO:8), wherein X, X₁, X₂ are any amino acid, wherein X, X₁, and X₂ can be the same or different amino acid, wherein n₁ and n₂ are equal to or greater than one. In one aspect, the polypeptide has the sequence selected from at least one of [(X₁X₂GXP)(X₃X₄GXP)]ₙ₁, [(X₁X₂GXP)ₙ₁(X₃X₄GXP)ₙ₂], or [(X₁X₂GXP)ₙ₁(X₃X₄GXP)ₙ₂(X₁X₂GXP)ₙ₁], wherein X, X₁, X₂, X₃, and X₄ are any amino acid, and nₙ₁ and n₂ are greater than or equal to one; wherein X₁ and X₂ can be the same or different from each other, and X₃ and X₄ can be the same or different from each other; or wherein at least one of X₁ or X₂ is different from X₃ or X₄, or wherein X is valine, or X₁=G, X₂=Y and A (in 1:4 ratio) and X=V. In another aspect, the polypeptide further comprises attaching to, or forming a fusion protein with, the polypeptide and at least a portion of an extracellular matrix component selected from at least one of: glycosaminoglycans (GAGs), proteoglycans, and/or proteins such as but not limited to laminin, fibronectin, vitronectin, collagen, elastin, fibrillin, fibulin, tenascin, perlecan, versican, aggrecan, neurocan, brevican, keratan, hyaluronic acid, heparan, or chondroitin, and wherein the fusion protein can be at an amino, a carboxy, or both the amino and carboxy ends of the polypeptide. In another aspect, the polypeptide further comprises attaching to, or forming a fusion protein with, the polypeptide and at least a portion of a growth factor or cytokine selected from at least one of: leukemia inhibitory factor, insulin, insulin like growth factors, epidermal growth factor, fibroblast growth factors including basic fibroblast growth factor, vascular endothelial growth factor, transforming growth factor-β, platelet-derived growth factor, neurotrophic factors, interleukin-2, stem cell factor, Fms-like tyrosine kinase 3/fetal liver kinase-2, granulocyte-macrophage colony-stimulating factor, interleukin 1 alpha, or granulocyte colony-stimulating factor, and wherein the fusion protein can be at an amino, a carboxy, or both the amino and carboxy ends of the polypeptide. In another aspect, the polypeptide further comprises attaching to, or forming a fusion protein with, the polypeptide and at least a portion of a growth factor/cytokine and an extracellular matrix component, wherein the fusion proteins can be at an amino, a carboxy, or both the amino and carboxy ends of the polypeptide. In another aspect, the polypeptide is provided in solution, attached to a substrate, or both. In another aspect, the polypeptide is a fusion protein with an amino-terminal end that comprises a laminin domain and a carboxy-terminal end comprises an elastin domain. In another aspect, the polypeptide comprises at least one of: (1) a laminin domain comprising one or more VGKKKKKKKKG motifs (SEQ ID NO: 3); (2) one or more YIGSRVGKKKKKKKKG motifs (SEQ ID NO: 6); (3) one or more RNAIAEIIKDI motifs (SEQ ID NO: 2); (4) an elastin domain comprising one or more [(GAGVP)₂(GYGVP)(GAGVP)₂]₁₂ motifs (SEQ ID NO: 4); (5) [(GAGVP)₂(GYGVP)(GAGVP)₂]₂₄ motifs (SEQ ID NO: 5); (6) SEQ ID NOS: 1 and 4, (7) SEQ ID NOS: 1 and 5; (8) SEQ ID NOS: 3 and 4; (9) SEQ ID NOS: 3 and 5; (10) SEQ ID NOS: 2 and 4, or (6) SEQ ID NOS: 2 and 5; (11) any combination of SEQ ID NOS: 1, 2, 3, 4, or 5, wherein the polypeptide has the sequence selected from at least one of [(X₁X₂GXP)(X₃X₄GXP)]ₙ₁, [(X₁X₂GXP)ₙ₁(X₃X₄GXP)ₙ₂]; (12) [(X₁X₂GXP)ₙ₁(X₃X₄GXP)ₙ₂(X₁X₂GXP)ₙ₁], wherein X₁, X₂, X₃, X₄, and X are any amino acid except proline; (13) [(X₁X₂GXP)(X₃X₄GXP)]ₙ₁, [(X₁X₂GXP)ₙ₁(X₃X₄GXP)ₙ₂]; or (14) [(X₁X₂GXP)ₙ₁(X₃X₄GXP)ₙ₂(X₁X₂GXP)ₙ₁], wherein X₁, X₂, X₃, X₄ is any amino acid and X is an aliphatic amino acid.

In another embodiment, the present invention includes a host cell that comprises a nucleic acid vector that encodes a polypeptide that comprises one or more repeats of a sequence n₁-(X₁X₂GXP)-n₂ (SEQ ID NO:8), wherein X, X₁, X₂ are any amino acid, wherein X, X₁, and X₂ can be the same or different amino acid, wherein n₁ and n₂ are equal to or greater than one. In one aspect, the host cell expresses or secretes the polypeptide.

In another embodiment, the present invention includes a method of making a fusion protein comprising: providing a host cell with a nucleic acid vector that expresses a polypeptide that comprises one or more repeats of a sequence n₁-(X₁X₂GXP)-n₂ (SEQ ID NO:8), wherein X, X₁, X₂ are any amino acid, wherein X, X₁, and X₂ can be the same or different amino acid, wherein n₁ and n₂ are equal to or greater than one, and wherein X is an aliphatic amino acid; and isolating the polypeptide. In one aspect, the polypeptide has the sequence selected from at least one of [(X₁X₂GXP)(X₃X₄GXP)]ₙ₁, [(X₁X₂GXP)ₙ₁(X₃X₄GXP)ₙ₂], or [(X₁X₂GXP)ₙ₁(X₃X₄GXP)ₙ₂(X₁X₂GXP)ₙ₁], wherein X, X₁, X₂, X₃, and X₄ are any amino acid, and n₁ and n₂ are greater than or equal to one; wherein X₁ and X₂ can be the same or different from each other, and X₃ and X₄ can be the same or different from each other; or wherein at least one of X₁ or X₂ is different from X₃ or X₄, or wherein X is valine, or X₁=G, X₂=Y and A (in 1:4 ratio) and X=V. In another aspect, the polypeptide further comprises attaching to, or forming a fusion protein with, the polypeptide and at least a portion of an extracellular matrix component selected from at least one of: glycosaminoglycans (GAGs), proteoglycans, and/or proteins such as but not limited to laminin, fibronectin, vitronectin, collagen, elastin, fibrillin, fibulin, tenascin, perlecan, versican, aggrecan, neurocan, brevican, keratan, hyaluronic acid, heparan, or chondroitin, and wherein the fusion protein can be at an amino, a carboxy, or both the amino and carboxy ends of the polypeptide. In another aspect, the polypeptide further comprises attaching to, or forming a fusion protein with, the polypeptide and at least a portion of a growth factor or cytokine selected from at least one of: leukemia inhibitory factor, insulin, insulin like growth factors, epidermal growth factor, fibroblast growth factors including basic fibroblast growth factor, vascular endothelial growth factor, transforming growth factor-β, platelet-derived growth factor, neurotrophic factors, interleukin-2, stem cell factor, Fms-like tyrosine kinase 3/fetal liver kinase-2, granulocyte-macrophage colony-stimulating factor, interleukin 1 alpha, or granulocyte colony-stimulating factor, and wherein the fusion protein can be at an amino, a carboxy, or both the amino and carboxy ends of the polypeptide. In another aspect, the polypeptide further comprises attaching to, or forming a fusion protein with, the polypeptide and at least a portion of a growth factor/cytokine and an extracellular matrix component, wherein the fusion proteins can be at an amino, a carboxy, or both the amino and carboxy ends of the polypeptide. In another aspect, the polypeptide is provided in solution, attached to a substrate, or both. In another aspect, the polypeptide is a fusion protein with an amino-terminal end that comprises a laminin domain and a carboxy-terminal end comprises an elastin domain. In another aspect, the polypeptide comprises at least one of: (1) a laminin domain comprising one or more VGKKKKKKKKG motifs (SEQ ID NO: 3); (2) one or more YIGSRVGKKKKKKKKG motifs (SEQ ID NO: 6); (3) one or more RNAIAEIIKDI motifs (SEQ ID NO: 2); (4) an elastin domain comprising one or more [(GAGVP)₂(GYGVP)(GAGVP)₂]₁₂ motifs (SEQ ID NO: 4); (5) [(GAGVP)₂(GYGVP)(GAGVP)₂]₂₄ motifs (SEQ ID NO: 5); (6) SEQ ID NOS: 1 and 4, (7) SEQ ID NOS: 1 and 5; (8) SEQ ID NOS: 3 and 4; (9) SEQ ID NOS: 3 and 5; (10) SEQ ID NOS: 2 and 4, or (6) SEQ ID NOS: 2 and 5; (11) any combination of SEQ ID NOS: 1, 2, 3, 4, or 5, wherein the polypeptide has the sequence selected from at least one of [(X₁X₂GXP)(X₃X₄GXP)]ₙ₁ (SEQ ID NO:68), [(X₁X₂GXP)ₙ₁(X₃X₄GXP)ₙ₂] (SEQ ID NO:69); (12) [(X₁X₂GXP)ₙ₁(X₃X₄GXP)ₙ₂(X₁X₂GXP)ₙ₁] (SEQ ID NO:70), wherein X₁, X₂, X₃, X₄, and X are any amino acid except proline; (13) [(X₁X₂GXP)(X₃X₄GXP)]ₙ₁ (SEQ ID NO:71), [(X₁X₂GXP)ₙ₁(X₃X₄GXP)ₙ₂] (SEQ ID NO:72); or (14) [(X₁X₂GXP)ₙ₁(X₃X₄GXP)ₙ₂(X₁X₂GXP)ₙ₁] (SEQ ID NO:73), wherein X₁, X₂, X₃, X₄ is any amino acid and X is an aliphatic amino acid. The method of claim 12, further comprising the step of forming a 3D cell culture system, wherein the polypeptide creates a 3D scaffold for cell growth. In another aspect, the polypeptide is dissolved at a temperature below Tₜ before use. In another aspect, the polypeptide is a recycled laminin-elastin motif protein (LEMP) prepared by: cycling the temperature of the LEMP above and below Tₜ such that the LEMP is at least one of (i) precipitated, (ii) washed, (iii) redissolved, and optionally steps (i) to (iii) can be repeated to remove impurities.

In another embodiment, the present invention includes a method of making cardiomyocytes comprising: seeding stem cells and incubating in a media that comprise a polypeptide that comprises one or more repeats of a sequence n₁-(X₁X₂GXP)-n₂ (SEQ ID NO:8), wherein X, X₁, X₂ are any amino acid, wherein X₁, X₂ and X can be the same or different amino acid, wherein n₁ and n₂ are equal to or greater than one, in stem cell media or coated on a surface of a substrate; culturing the stem cells without an anti-differentiation factor; changing the media to cardiac differentiation media; and isolating beating cardiomyocytes. In one aspect, the method further comprises attaching to, or forming a fusion protein with, the polypeptide and at least a portion of an extracellular matrix component selected from at least one of: glycosaminoglycans (GAGs), proteoglycans, and/or proteins such as but not limited to laminin, fibronectin, vitronectin, collagen, elastin, fibrillin, fibulin, tenascin, perlecan, versican, aggrecan, neurocan, brevican, keratan, hyaluronic acid, heparan, or chondroitin, and wherein the fusion protein can be at an amino, a carboxy, or both the amino and carboxy ends of the polypeptide. In another aspect, the method further comprises attaching to, or forming a fusion protein with, the polypeptide and at least a portion of a growth factor or cytokine selected from at least one of: leukemia inhibitory factor, insulin, insulin like growth factors, epidermal growth factor, fibroblast growth factors including basic fibroblast growth factor, vascular endothelial growth factor, transforming growth factor-β, platelet-derived growth factor, neurotrophic factors, interleukin-2, stem cell factor, Fms-like tyrosine kinase 3/fetal liver kinase-2, granulocyte-macrophage colony-stimulating factor, interleukin 1 alpha, or granulocyte colony-stimulating factor, and wherein the fusion protein can be at an amino, a carboxy, or both the amino and carboxy ends of the polypeptide. In another aspect, the method further comprises attaching to, or forming a fusion protein with, the polypeptide and at least a portion of a growth factor/cytokine and an extracellular matrix component, wherein the fusion proteins can be at an amino, a carboxy, or both the amino and carboxy ends of the polypeptide. In another aspect, the polypeptide is provided in solution, attached to a substrate, or both. In another aspect, the polypeptide comprises at least one of: (1) a laminin domain comprising one or more VGKKKKKKKKG motifs (SEQ ID NO: 3); (2) one or more YIGSRVGKKKKKKKKG motifs (SEQ ID NO: 6); (3) one or more RNAIAEIIKDI motifs (SEQ ID NO: 2); (4) an elastin domain comprising one or more [(GAGVP)₂(GYGVP)(GAGVP)₂]₁₂ motifs (SEQ ID NO: 4); (5) [(GAGVP)₂(GYGVP)(GAGVP)₂]₂₄ motifs (SEQ ID NO: 5); (6) SEQ ID NOS: 1 and 4, (7) SEQ ID NOS: 1 and 5; (8) SEQ ID NOS: 3 and 4; (9) SEQ ID NOS: 3 and 5; (10) SEQ ID NOS: 2 and 4, or (6) SEQ ID NOS: 2 and 5; (11) any combination of SEQ ID NOS: 1, 2, 3, 4, or 5, wherein the polypeptide has the sequence selected from at least one of [(X₁X₂GXP)(X₃X₄GXP)]ₙ₁, [(X₁X₂GXP)ₙ₁(X₃X₄GXP)ₙ₂]; (12) [(X₁X₂GXP)ₙ₁(X₃X₄GXP)ₙ₂(X₁X₂GXP)ₙ₁], wherein X₁, X₂, X₃, X₄, and X are any amino acid except proline; (13) [(X₁X₂GXP)(X₃X₄GXP)]ₙ₁, [(X₁X₂GXP)ₙ₁(X₃X₄GXP)ₙ₂]; or (14) [(X₁X₂GXP)ₙ₁(X₃X₄GXP)ₙ₂(X₁X₂GXP)ₙ₁], wherein X₁, X₂, X₃, X₄ is any amino acid and X is an aliphatic amino acid. In another aspect, the cardiac differentiation media does not include differentiation factors. In another aspect, the polypeptide is provided in a media at the same time as cells to be grown in the media or on a substrate. In another aspect, the cells for growth in a 3D culture system are primary cells, cell clones, cell lines, immortal cells, totipotent cells, multipotent cells, pluripotent cells, unipotent cells, stem cells, differentiated cells, or terminally differentiated cells. In another aspect, the cells are human cells. In another aspect, a substrate is a cell culture plate that comprises 1, 2, 4, 6, 8, 12, 16, 24, 32, 36, 48, 96, 192, or 384-well plates. In another aspect, the cardiac differentiation media comprises at least one of: RA (retinoic acid); AA (Ascorbic acid); FGF8 (Fibroblast growth factor 8); SHH (Sonic hedgehog); bFGF (basic Fibroblast growth factor); BDNF (Brain-derived neurotrophic factor); GDNF (Glial cell-derived neurotrophic factor; CHIR99021 (Glycogen synthase kinase 3(GSK-3) Inhibitor); or cAMP (Cyclic adenosine monophosphate).

In another embodiment, the present invention includes a beating cardiomyocyte made by a method comprising: seeding embryonic stem cells in a media comprising a polypeptide that comprises one or more repeats of a sequence n₁-(X₁X₂GXP)-n₂ (SEQ ID NO:8), wherein X, X₁, X₂ are any amino acid, wherein X₁, X₂ and X can be the same or different amino acid, wherein n₁ and n₂ are equal to or greater than one, in embryonic stem cell media; culturing the stem cells without an anti-differentiation factor; changing the media to cardiac differentiation media; and isolating beating cardiomyocytes.

In another embodiment, the present invention includes a method of making a 3D cell culture comprising: seeding cells and incubating in a media that comprises a polypeptide that comprises one or more repeats of a sequence n₁-(X₁X₂GXP)-n₂ (SEQ ID NO:8), wherein X, X₁, X₂ are any amino acid, wherein X₁, X₂ and X can be the same or different amino acid, wherein n₁ and n₂ are equal to or greater than one, in cell media or coated on the surface of culture substrate; culturing the stem cells with one or more growth factors; changing the media; and isolating the cells. In one aspect, the cells for growth in the 3D system are primary cells, cell clones, cell lines, immortal cells, cancer cells, totipotent cells, multipotent cells, pluripotent cells, unipotent cells, stem cells, differentiated cells, or terminally differentiated cells. In another aspect, the cells are human cells. In another aspect, the cells are viruses, bacterial cells, fungal cells, mammalian cells, insect cells, or plant cells. In another aspect, the polypeptide comprising a sequence (X₁X₂GVP)ₙ as a building block, where X₁ and X₂ are any amino acids except proline, and wherein X₁ and X₂ can be the same or different amino acids and wherein n is equal to or greater than one, wherein the polypeptide promotes cell growth in three dimensions. In another aspect, the method further comprises attaching to, or forming a fusion protein with, the polypeptide and at least a portion of an extracellular matrix component selected from at least one of: glycosaminoglycans (GAGs), proteoglycans, and/or proteins such as but not limited to laminin, fibronectin, vitronectin, collagen, elastin, fibrillin, fibulin, tenascin, perlecan, versican, aggrecan, neurocan, brevican, keratan, hyaluronic acid, heparan, or chondroitin, and wherein the fusion protein can be at an amino, a carboxy, or both the amino and carboxy ends of the polypeptide. In another aspect, the method further comprises attaching to, or forming a fusion protein with, the polypeptide and at least a portion of a growth factor or cytokine selected from at least one of: leukemia inhibitory factor, insulin, insulin like growth factors, epidermal growth factor, fibroblast growth factors including basic fibroblast growth factor, vascular endothelial growth factor, transforming growth factor-β, platelet-derived growth factor, neurotrophic factors, interleukin-2, stem cell factor, Fms-like tyrosine kinase 3/fetal liver kinase-2, granulocyte-macrophage colony-stimulating factor, interleukin 1 alpha, or granulocyte colony-stimulating factor, and wherein the fusion protein can be at an amino, a carboxy, or both the amino and carboxy ends of the polypeptide. In another aspect, the method further comprises attaching to, or forming a fusion protein with, the polypeptide and at least a portion of a growth factor/cytokine and an extracellular matrix component, wherein the fusion proteins can be at an amino, a carboxy, or both the amino and carboxy ends of the polypeptide. In another aspect, the one or more growth factors are selected from at least one of: RA (retinoic acid); BMP4 (Bone morphogenetic protein; Activin A; bFGF (basic Fibroblast growth factor); VEGF (Vascular endothelial growth factor); AA (Ascorbic acid); CHIR99021 (Glycogen synthase kinase 3(GSK-3) Inhibitor); or DKK1 (Dickkopf-related protein 1).

In another embodiment, the present invention includes a 3D cell culture system comprising: a substrate; and a polypeptide that comprises one or more repeats of a sequence n₁-(X₁X₂GXP)-n₂ (SEQ ID NO:8), wherein X, X₁, X₂ are any amino acid, wherein X₁, X₂ and X can be the same or different amino acid, wherein n₁ and n₂ are equal to or greater than one, wherein the polypeptide promotes cell growth in three dimensions. In one aspect, the polypeptide comprises a sequence (X₁X₂GVP)ₙ as a building block, where X₁ and X₂ are any amino acids except proline, and wherein X₁ and X₂ can be the same or different amino acids and wherein n is equal to or greater than 1. In another aspect, the polypeptide is mixed in a media or attached or adhered to the substrate. In another aspect, the polypeptide promotes totipotency, pluripotency, multipotency, or unipotency. In another aspect, the substrate is a gelatin-coated dish. In another aspect, the polypeptide is provided in a media at the same time as cells to be grown in the system. In another aspect, the one or more cells for growth in the 3D system are primary cells, cell clones, cell lines, immortal cells, cancer cells, totipotent cells, multipotent cells, pluripotent cells, unipotent cells, stem cells, differentiated cells, or terminally differentiated cells. In another aspect, the cells grown in three dimensions are human cells. In another aspect, the substrate is a cell culture plate that comprises 1, 2, 4, 6, 8, 12, 16, 24, 32, 36, 48, 96, 192, or 384-well plates. In another aspect, the substrate is charged with a positive or negative charge. In another aspect, the substrate is selected from at least one of polystyrene, polypropylene, polymethyl methacrylate, polyvinyl chloride, polymethyl pentene, polyethylene, polycarbonate, polysulfone, polystyrene, fluoropolymers, polyamides, or silicones. In another aspect, the system further comprises a thixotropic agent. In another aspect, a single building block sequence is used, that is the sequence of polypeptide is (X₁X₂GVP)ₙ, and n is greater than or equal to zero. In another aspect, the more than one different type of building block is joined in any order to construct the polypeptide comprising [(X₁X₂GVP)(X₃X₄GVP)]ₙ₁, [(X₁X₂GVP)ₙ₁(X₃X₄GVP)ₙ₂], or [(X₁X₂GVP)ₙ₁(X₃X₄GVP)ₙ₂(X₁X₂GVP)ₙ₁], wherein X₁, X₂, X₃, and X₄ are any amino acid except proline, and n₁ and n₂ are greater than or equal to one, or X₁=G, X₂=Y and A (in 1:4 ratio) and X=V. In another aspect, X₁ and X₂ can be the same or different from each other, and X₃ and X₄ can be the same or different from each other, however, at least one of X₁ or X₂ is different from X₃ or X₄ to obtain different building blocks. In another aspect, the polypeptide is attached to or a fusion protein with an extracellular matrix component selected from at least one of: glycosaminoglycans (GAGs), proteoglycans, or proteins. In another aspect, the system further comprises attaching to, or forming a fusion protein with, the polypeptide and at least a portion of an extracellular matrix component selected from at least one of: glycosaminoglycans (GAGs), proteoglycans, and/or proteins such as but not limited to laminin, fibronectin, vitronectin, collagen, elastin, fibrillin, fibulin, tenascin, perlecan, versican, aggrecan, neurocan, brevican, keratan, hyaluronic acid, heparan, or chondroitin, and wherein the fusion protein can be at an amino, a carboxy, or both the amino and carboxy ends of the polypeptide. In another aspect, the system further comprises attaching to, or forming a fusion protein with, the polypeptide and at least a portion of a growth factor or cytokine selected from at least one of: leukemia inhibitory factor, insulin, insulin like growth factors, epidermal growth factor, fibroblast growth factors including basic fibroblast growth factor, vascular endothelial growth factor, transforming growth factor-β, platelet-derived growth factor, neurotrophic factors, interleukin-2, stem cell factor, Fms-like tyrosine kinase 3/fetal liver kinase-2, granulocyte-macrophage colony-stimulating factor, interleukin 1 alpha, or granulocyte colony-stimulating factor, and wherein the fusion protein can be at an amino, a carboxy, or both the amino and carboxy ends of the polypeptide. In another aspect, the system further comprises attaching to, or forming a fusion protein with, the polypeptide and at least a portion of a growth factor/cytokine and an extracellular matrix component, wherein the fusion proteins can be at an amino, a carboxy, or both the amino and carboxy ends of the polypeptide.

### BRIEF DESCRIPTION OF THE DRAWINGS

For a more complete understanding of the features and advantages of the present invention, reference is now made to the detailed description of the invention along with the accompanying figures and in which:

FIGS. 1A-B. Show the concept of a using suspended extracellular matrix (ECM) blocks to support the growth of 3D cell cultures. ECM blocks should have a degree of flexibility to accommodate cell growth and be easy to separate from the cells.

FIGS. 2A-B. Laminin and elastin motifs used to make laminin-elastin motif proteins (LEMPs). (FIG. 2A) is a schematic showing LEMP design. Details of motifs that the present inventors have selected are given in the table. Motifs YIGSR^{27 28} (SEQ ID NO:1) and RNIAEIIKDI²⁹ (SEQ ID NO:2) have been shown to help in cell attachment and neurite growth. VGKKKKKKKKG (SEQ ID NO:3) was designed because polylysine has been shown to enhance cell attachment of many different cell types³⁸. (FIG. 2B) The transition temperatures (Tt) of the different LEMPs are given. E24 based LEMPs have Tt less than 37 C and are expected to form visible aggregates. Tt is the temperature where the optical density (O.D.) suddenly begins to increase rapidly.

FIG. 3. Comparison of gelatin-coated and LEMP-coated dishes for mouse embryonic stem cell (mESC) 2D culture. Coating of R E12 on a culture dish (5 μM, 37° C., 1 hr, washing twice, followed by addition of single mESCs) leads to 2D stem cell culture similar to a gelatin-coated surface at day 4 with leukemia inhibitory factor (LIF). In the absence of LIF, as expected, mESCs grown on both gelatin and R E12 coated dish start to differentiate.

FIGS. 4A-D. Successful 3D culture of mESCs and maintenance of pluripotency markers. (FIG. 4A) Morphology of mESCs in 3D culture system (passage #5) using different LEMPs. Note that while imaging the cells are brought out of 37° C. environment. As a result the temperature of the culture starts to drop, and R E24 for which Tt=28° C., it starts to change phase from solid aggregated state to dissolved state. (FIG. 4B) Quantitative real time PCR (qRT-PCR) analysis of Oct4 and Nanog expression in mESCs. Oct4 and Nanog, which are markers of SC pluripotency were detected at passage #1 and #10 to evaluate long term maintenance of mESC pluripotency when cultured in 3D. All data shown are mean±SD from the values of three replicates. (FIG. 4C) Immunocytochemistry of protein expression of pluripotency marker Oct4 of mESCs grown in 3D culture system (passage #7). Nuclei were stained with DAPI. Scale bars, 25 μm. (FIG. 4D) Flow cytometric analysis (FACS) of the pluripotency surface marker SSEA-1 for the mESCs (passage #8) grown in the LEMP 3D culture. FACS analysis shows that more than 95% of the cells grown in 3D culture groups examined are strongly positive for SSEA1.

FIG. 5. When no LEMPs are added to the culture media, mESCs after several passages exhibit big aggregates of ESCs and morphology is not spheroidal.

FIG. 6. LEMPs attach to ESCs and directly interact with SC spheroids. The present inventors imaged 3D SC spheroids under white light microscope. In the case of LEMPs based on elastin motif E12 (called LEMP 12 here) the LEMPs can be seen (left image) at the bottom of the dish and are harder to visualize on spheroid surfaces. However, LEMPs based on elastin motif E24 (called LEMP 24 here) can be easily visualized and can be seen attached (arrow in middle and right images) to the ESC spheroids.

FIGS. 7A-B. LEMP R E12 enables differentiation of mESCs into motor neurons by simple addition to media without use of laminin coated dishes. (FIG. 7A) Immunocytochemistry of Tuj1 neural marker protein expression was done. The differentiated mESCs were stained using specific antibodies against the marker Tuj1. A large number of cells showing neuronal morphology (Tuj1) were detected in R E12 addition group. Importantly a semi-3D (spheroids attached to plate surface) was seen in LEMP group. (FIG. 7B) Quantitative real time PCR analysis of neural marker gene expression for Nestin and Tuj1 showed that R E12 LEMP at different concentrations induced significantly higher expression for both Nestin and Tuj1 as compared to conventional ‘laminin-coating’ differentiation protocol. **: p<0.01. Method details: A published protocol was followed⁴³. Laminin-coated protocol: Briefly, single cell mESCs were added to laminin-coated plates in neuronal induction medium consisting of DMEM/F12, supplemented with growth factors (GFs) for 5 days. On day 5, retinoic acid (1 μM) and 500 ng/mL sonic hedgehog were added from days 5 to 12. On day 12, neuronal progenitors were cultured in neurobasal medium with GFs. After 14 more days of culture cells were either stained or subjected to qRT-PCR. LEMP protocol: Uncoated dishes were used. The same workflow and media as described for laminin-coatings was used, except LEMP (R E12 or R E24) was added at the time of media change, which was done every 2 days.

FIGS. 8A-D. LEMPs enable differentiation of mESCs into dopaminergic neurons as semi-3D spheroids. (FIGS. 8A, 8B) Total RNA was extracted from each LEMP treated group and control (laminin coated dish), and quantitative real time PCR analysis of neural (Tuj1) and dopaminergic neuron (Tyrosine hydroxylase=TH) marker gene expression was done after 20 days of differentiation. (FIG. 8C) Immunocytochemistry for protein expression of dopaminergic neuron marker (TH, Red) and neurons (Tuj1, green) was done at day 20 after differentiation. FIG. 8D: PROTOCOL DETAILS: The present inventors followed the method described previously(44). Briefly, embryoid body (EB) was formed. On the 4th day, the EBs were collected, dissociated, and either (i) plated on 0.1% gelatin-coated dishes (control), or (ii) plated on LEMP-coated dishes (10 μM, 37° C., 1h), or (iii) added to uncoated dishes without any coating but with LEMPs (5 μM) LEMP-addition groups. To initiate neural differentiation, cells were cultured in DMEM/F-12 media containing neural N2 supplement for 7-9 days with media replacement every 1-2 days. For the LEMP-addition group fresh LEMP was added during these media changes. Next, cells were detached from plates of control (gelatin-coated) and LEMP-coated groups and plated onto a dish coated with laminin (for control group) or respective LEMP (for LEMP-coated group) at a density of 75,000 cells per cm2. For LEMP-addition groups the cells were continually cultured in the same dish without dissociation. After 24 hours, these neural cells were expanded further by changing to the DMEM/F12 medium supplemented with B27 supplement and several other factors such as bFGF, Sonic hedgehog, basic fibroblast growth factor 8b for 4 days. Terminal differentiation into dopaminergic neurons was performed by culturing these expanded neural cells in neuronal-expansion media (DMEM/F12 media containing ascorbic acid instead of bFGF) for 8-10 days. After 20 days of terminal differentiation, the present inventors performed analysis with qRT-PCR and Immunocytochemistry.

FIG. 9. Semi-3D spheroids of dopaminergic neurons are formed with use of LEMPs. With laminin-coated dish protocol, dopaminergic neurons largely exist in a planar format with some raised morphologies. In contrast, with LEMPs more and larger raised spheroidal morphologies were formed and these spheroids contained dopaminergic neurons in the internal volume as seen by confocal sectioning of the spheroid following immunostaining for neuronal marker Tuj-1 and dopaminergic neuron TH.

FIG. 10. LEMPs enable 3D culture of human ESCs. 4×10⁵ single H9 hESCs were seeded in non-adherent dishes (60 mm, 5 ml mTeSR™1 Medium), different LEMPs (8 μM) were added, and allowed to culture for 4 days. Cells were passaged as described for mESCs by first washing with PBS at room temperature, treating with accutase at 37° C. to dissociate 3D spheroids into single cells, which were then passaged. The present inventors examined the (top) size of spheroids, and (bottom) their morphology at passage #2.

FIG. 11 shows a comparison of a ‘general’ protocol of the prior art (top), compared to the ‘LEMP’ protocol for differentiation of the present invention (bottom).

FIGS. 12A and 12B show a differentiation protocol of cardiomyocytes from mESCs. (FIG. 12A) Schematic of EB-based cardiac differentiation. FIG. 12B Scheme of direct differentiation of mESCs into cardiomyocyte without EB formation.

FIG. 13 shows the MALDI-TOF spectra of the Y₁₂ ELP, with the calculated molecular weight.

FIGS. 14A to 14C show ELP characterization and cardiomyocyte differentiation rate from crosslinked ELP coated dishes. (FIG. 14A) Turbidity and Tts for 25 μM solutions of Y₁₂ and Y₂₄ ELPs (FIG. 14B) Cell viability of Y₁₂ and Y₂₄ ELPs at different concentration (microgram/ml). (FIG. 14C) Cardiomyocyte beating colony formation from EB based and direct differentiation protocol. Y₁₂ and Y₂₄ ELP was crosslinked overnight by tyrosinase before cell seeding. As comparison, non-crosslinked Y₁₂ and Y₂₄ were also used. Gelatin coated dish was used as a control. Effect of AA was also studied.

FIGS. 15A to 15D show the characterization of cardiomyocytes grown on the crosslinked ELP coated dishes. (FIG. 15A) Morphology. (FIG. 15B) Beating rate of cardiomyocytes on the crosslinked Y₁₂ ELP coated dishes. (FIG. 15C). SEM image of the crosslinked Y₁₂ and Y₂₄ ELP coated dishes. (FIG. 15D)Visualization of myocardial cell contraction using the calcium indicator Fluo-4. It is a representative image of resting and contracting cardiomyocytes that have taken up calcium inflow during beating. The mean of the contraction interval was determined by the time between low Fluo-4 fluorescence and high Fluo-4 fluorescence.

FIGS. 16A and 16B show immunostaining of cardiomyocytes. (FIG. 16A) Morphology of cardiomyocyte differentiation as time lapse (FIG. 16B) immunofluorescent staining of differentiated cardiomyocytes for troponin T cardiac isoform (cTnT2) and smooth muscle actin (SMA) at14 days after differentiation. Cell nuclei are stained with DAPI; D3 ES cells were seeded in gelatin coated dish as a control or crosslinked Y₁₂ ELP (75 μg/ml) .

FIGS. 17A to 17C show a microarray analysis of the cardiomyocytes of the present invention.

FIG. 18 shows the validation of microarray analysis. qRT-PCR analysis of each developmental stage cardiomyocyte marker expression. Mesoderm (MESP1) , cardiac progenitor (GATA4, ISL1, NKX_2.5, Mef2c and TBX5) and mature cardiomyocyte (cTNT2, Mlc2v, NPPA, NPPB, WT1 and TBX₁₈).

FIGS. 19A to 19E shows the effect of AA on cardiomyocytes differentiation. (FIG. 19A) beating rate of cardiomyocytes treated with AA in crosslinked Y₁₂ ELP coated dishes. (FIG. 19B) qRT-PCR analysis of cardiomyocyte marker gene expression of cTNT2 in each concentration of crosslinked Y₁₂ ELP in the presence of AA. (FIG. 19C, FIG. 19D) other lineage marker expression of each concentration of crosslinked Y₁₂ ELP coated dishes. (FIG. 19E) Immunostaining of cTNT2 protein expression in the AA treated Y₁₂ ELP crosslinked dish.

FIGS. 20A to 20E show the direct differentiation of mouse induced pluripotent stem cell line (derived from mouse embryonic fibroblast by the inventors and the cell line is named IPS#1) in crosslinked Y₁₂ ELP. (FIG. 20A) Beating colony fraction obtained from D3 (mouse ES cell line), and IPS #1 (mouse induced pluripotent cell) lines differentiated on the crosslinked Y₁₂ELP coated dishes. (FIG. 20B) Beating rate per minute of cardiomyocytes obtained from D3, and from IPS #1 cell lines differentiated on the crosslinked Y₁₂ ELP coated dishes. (FIG. 20C) Representative gene expression assays at each developmental stage. (FIG. 20D) Immunocytochemistry of cTnT2 expression in cardiomyocytes differentiated from D3 and from IPS #1 cells in cross-linked Y₁₂ ELP coated dishes. (FIG. 20E) FACS analysis of cTnT2 expression in cardiomyocytes obtained from D3 and from IPS #1 cells differentiated in cross-linked Y₁₂ ELP coated dishes.

FIGS. 21A and 21B. Schematic showing the protocol for direct differentiation of (FIG. 21A) mESCs and miPSCs, and (FIG. 21B) hESCs, into cardiomyocytes using crosslinked ELPs.

FIGS. 22A to 22D. ELP characterization and cardiomyocyte differentiation using D3 mESCs. (FIG. 22A) Turbidity and Tₜ for 25 μM solutions of crosslinked and non-crosslinked Y₁₂ and Y₂₄ ELPs. (FIG. 22B) Cell viability of D3 mESCs on crosslinked Y₁₂ and Y₂₄ coated dishes. (FIG. 22C) Proportion of cardiomyocyte beating colonies on non-crosslinked and crosslinked Y₁₂ and Y₂₄ ELP coated surfaces. Gelatin coated dish and non-coated (No coat) dishes were used as a control. Data are mean±SEM, n=3. ****: p<0.0001, **: p<0.05. (FIG. 22D) SEM image of surface of tissue culture plate coated Y₁₂ ELP with and without crosslinking. CL: crosslinked, N-CL: non-crosslinked

FIGS. 23A to 23G. Characterization of cardiomyocytes generated on crosslinked Y₁₂ (CL_Y₁₂) coated dishes using D3 mESCs. (FIG. 23A) Cell morphology of D3 mESCs on day 2 of culture. Scale bar=100 μm. (FIG. 23B) Beating rate of cardiomyocytes. The beating rate is presented in total counts of beating in cardiomyocytes per minute. BPM: beats per minute. Data are mean±SEM, n=5. n.s: not significant. (FIG. 23C) qRT-PCR gene expression pattern of cTNT2 (cardiomyocytes-specific marker), Tuj1 (ectoderm) and AFP (endoderm). (FIG. 23D) Confocal time lapse images of cardiomyocytes contraction on day 9 using the Ca²⁺ indicator Fluo-4 AM. The interval between the Ca²⁺ influx time was measured by Fluo-4 AM intensity for 30 sec and analyzed. sec: second. Scale bar=100 μm. (FIG. 23E) Average of Ca²⁺ peak time of cardiomyocytes based on the Fluo-4 AM intensity Green: Fluo-4 AM. Data are mean±SEM, n=3. (FIG. 23F) Immunofluorescent staining of differentiated cardiomyocytes for cTNT2 and SMA expression on day 14 after differentiation on LN521 and CL_Y₁₂(75 μg/ml) coated dishes without AA. Scale bar=100 82 m. Cell nuclei are stained with DAPI. Scale bar=25 μm. (FIG. 23G) FACS analysis of cTNT2 expression in cardiomyocytes differentiated from D3 mESCs on CL_Y₁₂ (75 μg/ml) coated dishes without AA. gray color: isotype control. *: p<0.05.

FIGS. 24A to 24E. Gene analysis of cardiomyocytes differentiated from D3 mESCs without addition of exogenous factors. (FIG. 24A) Microarray analysis. A scatterplot of log 2 differentially expressed genes (DEGs) at day 9 and day 14 of cardiac differentiation. (FIG. 24B) GO and KEGG enrichment (FDR 0.05) analyses of genes upregulated at day 9 and 14 of cardiac differentiation, identifying heart development, focal/cell adhesion, and ECM genes among the top pathways. (FIG. 24C) Heatmap of fold-changes in the expression of cardiomyocyte marker genes at day 9 and 14 of cardiomyocytes induction (FDR 0.05). (FIG. 24D) Gene set enrichment analysis (GSEA) shows positive correlation of cardiomyocyte (left) and ECM (right) genes at day 9 or 14 of differentiation. (FIG. 24E) Heatmap of fold-changes in the expression of ECM marker genes on CL_Y₁₂ coated dish in comparison to mESCs at day 9 and 14 of cardiomyocyte induction (FDR 0.05). FDR: False Discovery Rate

FIGS. 25A to 25D. qRT-PCR analysis of cardiomyocytes differentiated from D3 mESCs on CL_Y₁₂ at day 9 and 14 after cardiac differentiation. (FIG. 25A) mESCs markers: Oct4 and Nanog; (FIG. 25B) Mesoderm marker: Mesp1; (FIG. 25C) cardiac progenitor markers: Gata4, Isl1, Nkx2.5, Mef2c and Tbx5; and (FIG. 25D) cardiomyocyte maturation markers: cTNT2, Mlc2v, Nppa, Nppb, Wt1 and Tbx18. Data are normalized to glyceraldehyde 3-phosphate dehydrogenase gene (GAPDH). *: p<0.05, **: p<0.01.

FIGS. 26A to 26E. Effect of AA on cardiac differentiation of mESCs. D3 mESCs were differentiated in the absence (AA(−)) or presence of 100 μM AA (AA(+)) on CL_Y₁₂ELPs coated dishes on day 14, and resulting cardiomyocytes were characterized. (FIG. 26A) Beating rate. BPM: beats per minute. (FIG. 26B) Gene expression of cTNT2 (cardiomyocyte maturation marker) using qRT-PCR. (FIG. 26C, FIG. 26D) Gene expression of other lineage markers (Tuj1: ectoderm and AFP: endoderm) using qRT-PCR. (FIG. 26E) Immunostaining of cTNT2 protein expression in cardiomyocytes obtained from CL_Y₁₂ (75 μg/ml) coated dishes in the presence of AA. Scale bar=50 μm and 25 μm for low and high magnification, respectively. n.s: not significant, *: p<0.05.

FIGS. 27A to 27E. Direct differentiation of miPSCs on CL_Y₁₂ at 75 μg/ml. miPSC#1 and D3 mESCs were differentiated into cardiomyocytes on CL_Y₁₂. D3 mESCs were differentiated into cardiomyocytes as a control. (FIG. 27A) Percentage of beating colonies on day 9. (FIG. 27B) Beating rate of cardiomyocytes on day 9. BPM: beats per minute. (FIG. 27C) Gene expression of Mesp1: Mesoderm marker; cTNT2, TBX₁₈: cardiomyocyte maturation markers on day 14. #1: miPSCs #1, n.s: not significant, *: p<0.05. (FIG. 27D) Immunostaining of cTNT2 protein expression in cardiomyocytes (Green color) on day 14. Blue color: DAPI. Scale bar=25 μm. (FIG. 27E) FACS analysis of cTNT2 expression in cardiomyocytes. AA(−): without AA, AA(+): with 100 μM AA, Blue color: isotype control.

FIGS. 28A to 28E. Direct differentiation of H9 hESCs on CL_Y₁₂ at 75 μg/ml and Matrigel. (FIG. 28A) Change in morphology of hESCs at day 2, 4, 6 and 8 after commencement of differentiation. Scale bar=100 μm. (FIG. 28B) Beating rate of cardiomyocytes on day 9. BPM: beats per minute. (FIG. 28C) Gene expression of cTNT2, a cardiomyocyte maturation marker on day 14. (FIG. 28D) Immunostaining of cTNT2 and Actinin in cardiomyocytes differentiated from hESCs on day 14. Scale bar=25 μm. (FIG. 28E) Average of Ca2+ peak time of cardiomyocytes based on the Fluo-4 AM intensity Green: Fluo-4 AM. Data are mean±SEM, n=3. n.s: not significant.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Lack of a simple and reproducible method of generating either large quantities of cell clones, cells lines, primary cells, totipotent stem cells, pluripotent stem cells, multipotent stem cells, unipotent stem cells, collectively “stem cells” (SCs), or differentiated cells (collectively “cells”), such as, progenitor cells or somatic stem cells, is a major obstacle holding back the use of cell-based therapies, pluripotent-cell therapies, SC-based therapies, anti-cancer treatments, control over immune responses, tissue replacements, etc. In certain aspects, the cells are primary cells, cell clones, cell lines, immortal cells, cancer cells, totipotent cells, multipotent cells, pluripotent cells, unipotent cells, stem cells, differentiated cells, or terminally differentiated cells. For example, it has been estimated that 10⁹ cardiomyocytes are required to treat a patient with myocardial infarction, and 10¹⁰ SCs are required to screen a million molecules in a drug library. A 3D culture system is more suitable for growing large quantities of cells because in a 2D platform an enormous surface area would be required. Further, 3D cultures recapitulate the natural 3D niche of cells leading to improved cell growth and functionality. However, a simple 3D culture system for cells remains a major unmet need. To address this need, the present inventors have developed a novel biomaterial for 3D culture of cells, primary or immortalized. There is also a need for the development of, e.g., 3D scaffolds for pluripotent or stem cell growth substrates in which these cells are able to differentiate into different lineages by simply adding specific growth factors, etc., into the culture medium. By use of this biomaterial the present inventors have eliminated the cumbersome need to coat cell culture surfaces with laminin, matrigel or other biomaterials. This biomaterial was designed by recognizing that the extracellular matrix (ECM) components such as laminin, collagen, and elastin are critical for the growth of the embryo. Laminin is already being successfully used as a coating material during the differentiation stage of SCs. The present inventors postulated that to grow 3D cells, e.g., spheroids or even structured tissues, the ECM must be available in the 3D space so that it can interact with the spheroids, and it should be pliable to respond to the changing environment from continuous growth of the spheroids. The present inventors used elastin as a framework for the scaffold in the form of a novel fusion protein. The present invention uses a unique class of biopolymers called elastin-like proteins (ELPs). ELP's include motifs derived from the elastin sequence, which are repeated to form ELPs. An important property of ELP's is that they aggregate when their solution is heated, and the temperature at which they aggregate can be tuned by modifying the ELP design. The present inventors used suspended ELP aggregates as ECM scaffolds. The present inventors combined laminin motifs with ELPs to engineer a fusion protein, which the present inventors call laminin-elastin motif protein (LEMP). The present inventors shows that, (i) addition of LEMP to the culture media leads to a 3D culture for both mouse ESCs (mESCs) and human ESCs (hESCs), and (ii) addition of LEMP to the differentiation media for neuronal lineage forms motor neurons and dopaminergic neurons without the use of coatings. Thus, the LEMP-based 3D culture system developed allows for long term cell growth. In one non-limiting example, the LEMP-based 3D culture system allows for self-renewal of SCs and for their differentiation into the neuronal lineage with high yield.

### EXAMPLE 1

### Development of LEMP-Based 3D hESC Culture System

Development of LEMP-based 3D hESC culture system and characterize LEMP interaction with SCs. Different LEMP designs are screened to select candidate LEMP(s) that can enable long term 3D culture of hESCs (at least 50 passages) without causing their differentiation. The selected LEMPs are used to grow H9 hESC 3D cultures. Non-limiting examples of measures and assays that are used to optimize the LEMP-based 3D culture system include, e.g., cell viability, total SC yield, spheroid colony size, pluripotency markers (via immunocytochemistry and FACS), karyotyping, and in vivo teratoma formation are performed on these 3D cultures to further select lead LEMP candidates. Optimized methods are confirmed in one more hESC and one hiPSC line. To understand how LEMPs enable SC 3D cultures, but not a limitation of the present invention, it is possible to characterize the spheroid-LEMP system by fixing them and taking electron and light microscopy images. Microarray gene expression analysis and single-cell RNA sequencing of SCs cultured with or without LEMPs are performed to identify any changes induced in SCs by LEMPs. Energy metabolism (oxygen consumption rate and extracellular acidification rate) of 2D and 3D hESC cultures are compared to understand bioenergetics and mitochondrial activity, bioenergetics and other functions.

Development of LEMP-based system for hESCs differentiation into dopaminergic neurons. First, different LEMP designs are compared in their ability to generate dopaminergic neurons. Dopaminergic neuronal markers, cell yield, the amount of dopamine released, and in vitro electrophysiological recordings are used as criteria to select lead LEMP candidates. The selected LEMPs are further optimized for dose. Traditional 2D-derived and 3D cultured dopaminergic neurons are compared in vitro, especially for dopaminergic functionality, electrophysiology recordings, genomic stability (karyotyping), and mitochondrial bioenergetics, function, biogenesis and synaptic activity.

Assessment of the efficacy of LEMP-derived dopaminergic neurons in a Parkinson's disease model and evaluate if co-delivery of LEMP can enhance efficacy. Dopaminergic neurons derived from LEMP-based 3D differentiation method are injected in rat brain to assess their survival. Dopaminergic neurons derived from 2D protocol are used as a control. Brains are collected for immunohistochemistry of dopaminergic neuronal markers to determine identity of cells and to quantify dopaminergic cells per unit area. To test efficacy, 3D and 2D dopaminergic neurons will also be injected in a Parkinson's disease rat model. Rotational behavior test are done to evaluate treatment efficacy. Because LEMPs provide a nurturing environment for dopaminergic neurons in vitro, the present inventors can test if their co-delivery with dopaminergic neurons can provide the same growth stimulus and thus increase the therapeutic efficacy. Electrophysiology on brain slices are done to compare 2D and 3D cultured dopaminergic neurons.

A 3D cell culture system for PSCs. Large number of parent PSCs are required for in vivo therapy. PSCs have tremendous potential in cell-based therapies and tissue regeneration¹, drug discovery and toxicity², and organoid formation for use in basic research and finding treatments³. Already multiple companies are investigating human PSCs to develop treatments¹. However, large number of PSCs are required for these applications. For example, about 1-2×10⁹ cardiomyocytes are required to treat myocardial infarction (MI) in an adult weighing 50-100 kg⁴, about 1×10¹⁰ hepatocytes are required for hepatic failure⁵, and 1×10⁵ dopaminergic neurons are required for Parkinson's disease (PD) treatment⁶. These numbers are for one patient, and for millions of patients the numbers are staggering. It has been estimated that just for US patients with PD or MI, 2D surfaces in the order of 1-16 km² are required to grow the dopaminergic neurons and cardiomyocytes, not including the surface needed to grow the parent PSCs. A 3D culture on the other hand can achieve the same feat in a much smaller volume.

3D better simulates the natural in vivo niche and tissue environment. The natural environment of cells is 3D. PSCs are even more contact dependent, and they have been shown to exhibit improved qualities when grown in 3D. For example, pluripotency and osteoblast differentiation of mouse PSCs was found to be better in a 3D scaffold as compared to 2D culture⁷. In another example chondrogenesis of ESCs was better when cells were cultured in 3D embryoid bodies as compared to monolayer culture⁷. Thus, a 3D culture system is not only important to expand PSCs, but it is also important for their differentiation.

Current state of 3D culture systems for, for example, SCs. Materials such as atelocollagen⁷, hyaluronic acid⁸, thermoresponsive PNIPAAm-PEG polymer⁹, alginate^{10,11} have been used to create 3D scaffolds for culture of SCs. In other approaches bioreactors with hollow fiber capillary membrane system¹², stirred tank reactors^{13,14}, microcarriers^{15,16}, or even suspension cultures without microcarriers¹⁷ have been evaluated for 3D expansion of SCs. Despite successes, problems reported with these systems include formation of aggregates that reduce nutrient diffusion into and waste removal from the core leading to necrosis even when microcarriers are used¹⁵. Continuous stirring¹⁸ can be used to keep the size of 3D spheroids small, however, shear from agitation can reduce cell viability^{19,20}. With scaffolds, often times the difficulty arises when cells have to be recovered from the scaffolds. For example to dissolve alginate scaffolds, ethylenediaminetetraacetic acid (EDTA) was used¹¹. These additional cell-recovery steps introduce more unknowns that require optimization. Further, uncontrolled differentiation is also reported in the scaffolds²¹. Thus, there is need for a chemically-defined, simple, scalable, robust, and low-labor 3D cell culture expansion system.

3D PSC culture systems. Extracellular matrix is a key player in embryo development and stem cell culture. The extracellular matrix (ECM) plays a critical role in the development of the embryo²². Laminin, collagen, elastin, and fibronectin are some of the major components of the ECM. Their importance becomes self-evident if the present inventors focus on the loss-of-function phenotypes for these ECM components. For example, loss of β1 component of laminin is lethal to the embryo²³, loss of β2 of laminin leads to growth arrest and neuromuscular defects²⁴, and loss of elastin leads to postnatal death in 4 days²⁵. Matrigel®, which is now widely used as a support for SC culture is rich in laminin, collagen and other ECM proteins. Additionally, the ECM proteins, especially laminin has been shown to be a key regulator in stem cell pluripotency²⁶. Thus, clearly the ECM plays a significant role in stem cell renewal and differentiation.

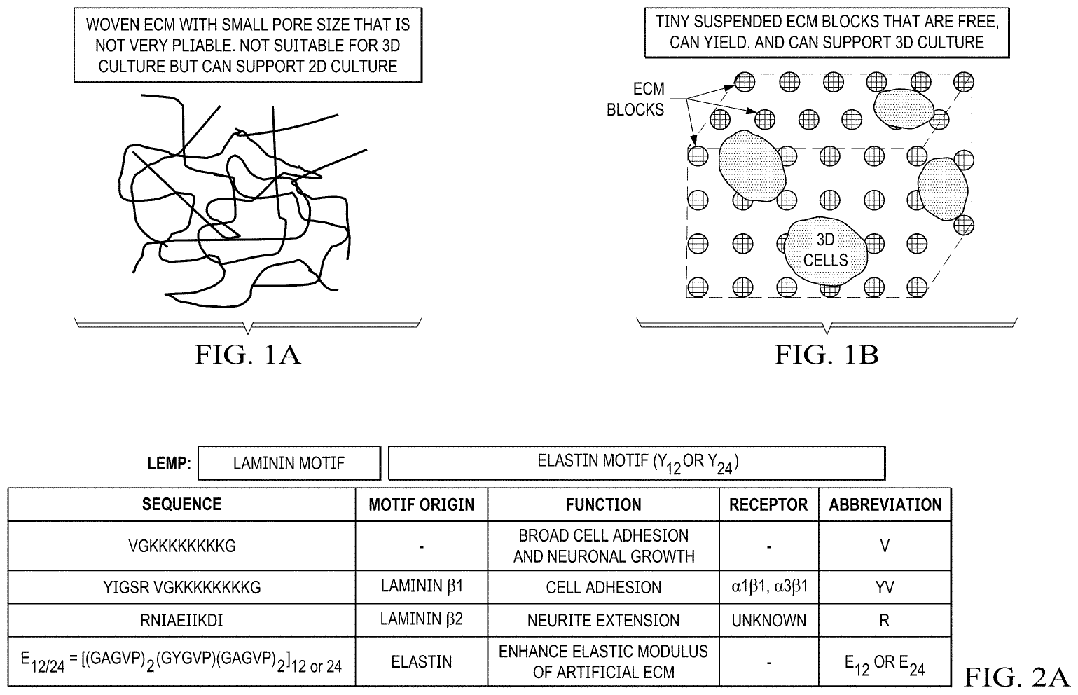

Suspended ECM blocks as a basis to support 3D cell culture. As shown in FIGS. 1A-B, show the woven ECM is suitable for 2D cell culture, but it is difficult to engineer a mesh that can fill the 3D space and can also yield to make room for the growing mass of 3D cells. In contrast, if ECM blocks were free, it is possible to fill the 3D space with them to support 3D cell growth. It is important however, that these ECM building blocks be biocompatible, and be easy to separate from the 3D culture when needed.

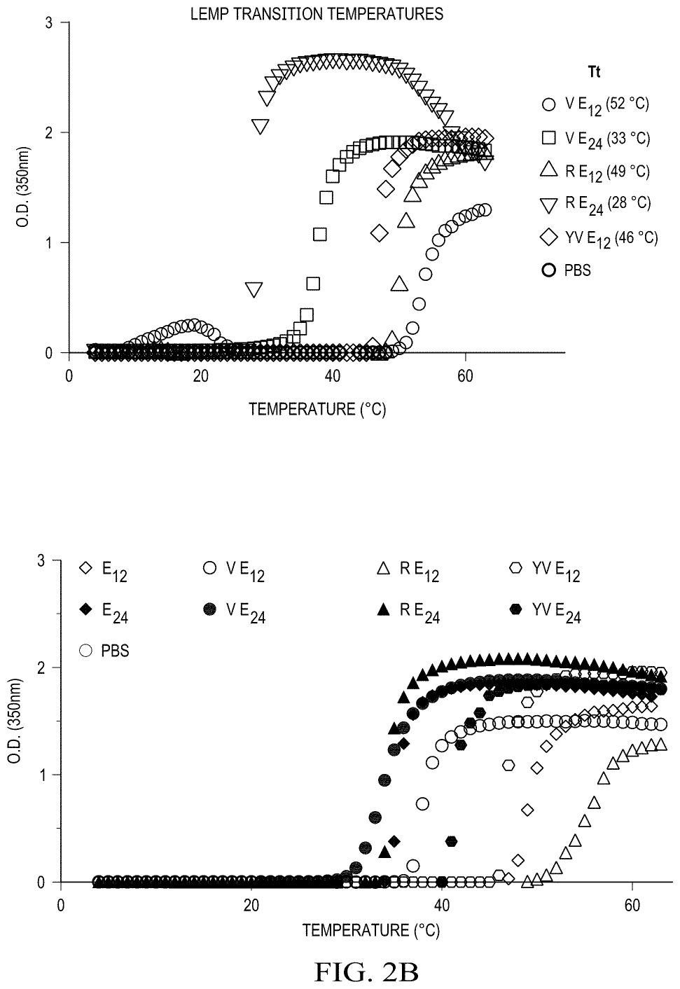

The design of the suspended ECM blocks: Laminin-Elastin Motif Protein (LEMP). The present inventors made a chimeric molecule or fusion protein that contains motifs from ECM components that can phase separate to form blocks. As shown in FIG. 2A, the designed molecule contains laminin and elastin motifs, and so the present inventors call it laminin-elastin motif protein (LEMP). The molecule is precisely defined and is made from biocompatible domains.

The present inventors searched the literature and identified laminin motifs that have previously been shown to help in cell growth and thus selected the laminin motifs YIGSR^{27 28} (SEQ ID NO:1) and RNIAEIIKDI²⁹ (SEQ ID NO:2). The amino acid (AA) motif ‘GXGX′P’ (X=any amino acid other than P:proline, and X′ is any aliphatic amino acid, but in some cases X′ is valine) (SEQ ID NO:4) is repeated 27 times in the 786 AA long human elastin molecule (UniProt: P15502), thus forming about 17% of the protein (5×27/786). It has been shown that when (GXGX′P)ₙ^{30,31} (SEQ ID NO:4) is repeated to form a large molecule, which is called elastin like protein (ELP), it shows unusual thermal properties. It is soluble in water below a certain temperature dubbed as the inverse transition temperature (Tₜ), but precipitates when the temperature is raised above Tₜ^{30,32}. The identity of ‘X’ in GXGX′P (SEQ ID NO:4) and the number of times GXGX′P (SEQ ID NO:4) is repeated (basically the length of ELP) plays an important role in determining the Tₜ³¹. ELPs are biocompatible and biodegradable, and have thus attracted much attention for drug delivery and tissue engineering applications^{30,32-36}. The thermal transition property allows it to be easily purified by thermal cycling to perform steps of precipitation, spinning, washing, and resolubilizing it to remove impurities³⁷. The present inventors designed the ELP so that it could phase separate below 37° C. Accordingly the present inventors selected ‘X’ to be hydrophobic and the repeating block was Y_{12 or 24}=[(GAGVP)₂(GYGVP)(GAGVP)₂]_{12 or 24} (see FIG. 2A) (SEQ ID NO:5). The Tₜ of the different LEMPs is shown in FIG. 2B. It can be seen that LEMPs based on Y₂₄ have Tₜ lower than 37° C. The present inventors also selected VGKKKKKKKKG (SEQ ID NO:2) as a motif because polylysine has been shown to enable attachment of multiple cell types. The present inventors hypothesized that this might help during neuronal differentiation.

Thus, a polypeptide for use in a three dimensional (3D) culture system for the growth of cells comprising one or more repeats of a sequence n₁-(X₁X₂GXP)-n₂, (SEQ ID NO:8) wherein X₁ and X₂ are any amino acids except proline, wherein X₁ and X₂ can be the same or different amino acid in solution or coated on a substrate, wherein n₁ and n₂ are equal to or greater than one, and wherein X is an aliphatic amino acid. In one specific example, X₁=G, X₂=Y and A (in 1:4 ratio) and X=V.

As used herein, the term “aliphatic amino acid” refers to glycine, alanine, valine, leucine or isoleucine, or equivalents thereof, including D and L-amino acids or amino acids that are, e.g., hydroxylated or acetylated.

Unique ECM blocks suspended in media to support 3D cell culture. The idea of using ECM blocks that are not crosslinked but are also not soluble is novel. By keeping the ECM blocks in a solid state as opposed to adding them as soluble molecules recapitulates the in vivo ECM state where it is in a solid state. By not crosslinking the blocks, the present inventors have allowed the ECM to yield and make space for the growing 3D spheroids.

LEMPs: chimeras that are easy to purify for synthesis, and easy to remove from cell culture. The present inventors have used elastin motifs, which are the basis of the ELP technology to create the unique ECM blocks. The present inventors have selected laminin motifs previously shown to be beneficial for cell culture and fused them to ELP motifs to create chimeras, which the present inventors call LEMPs (laminin-elastin motif proteins). Because ELPs have a unique ability to aggregate at temperatures higher than their transition temperatures (Tₜ), the present inventors have engineered the ELP motif to have a Tₜ<37° C. This causes spontaneous formation of LEMP aggregates at 37° C. Upon washing the cell culture with media cooler than Tₜ, the LEMPs redissolve and can be removed. Likewise, during production of LEMPs, cycling the temperature of the impure LEMP solution above and below Tₜ allows LEMP to be (i) precipitated, (ii) washed, (iii) redissolved, and steps (i) to (iii) can be repeated to remove impurities.

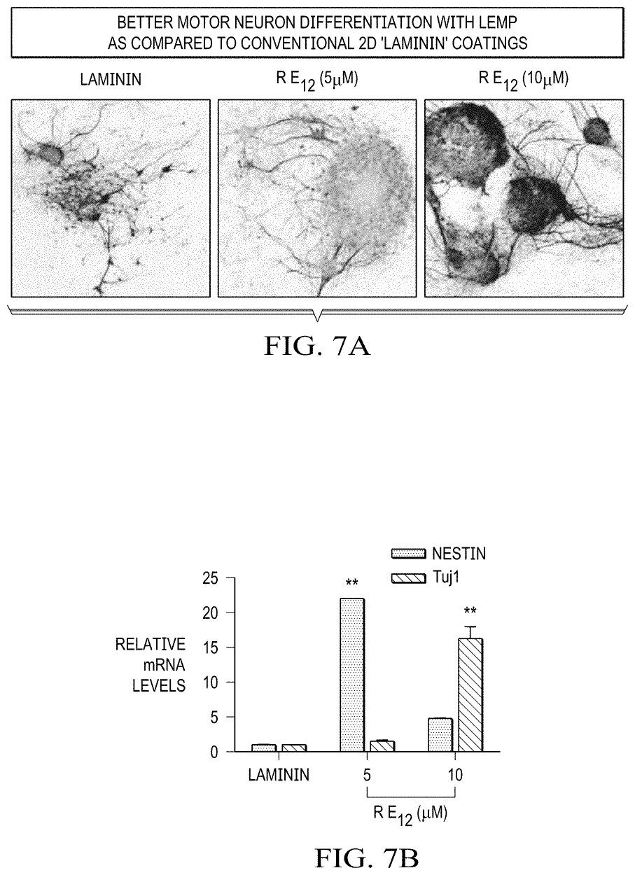

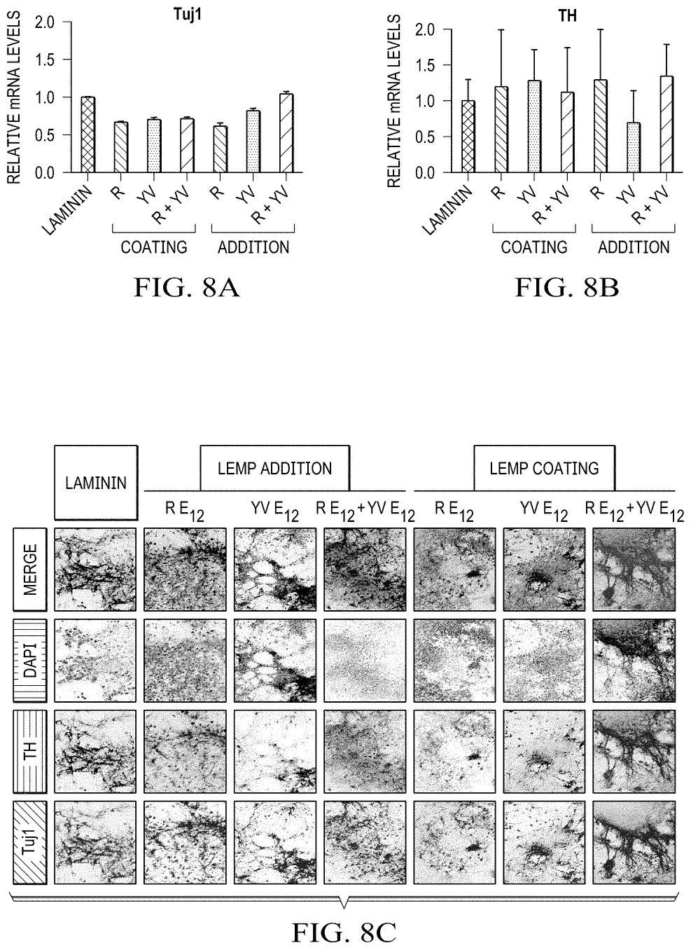

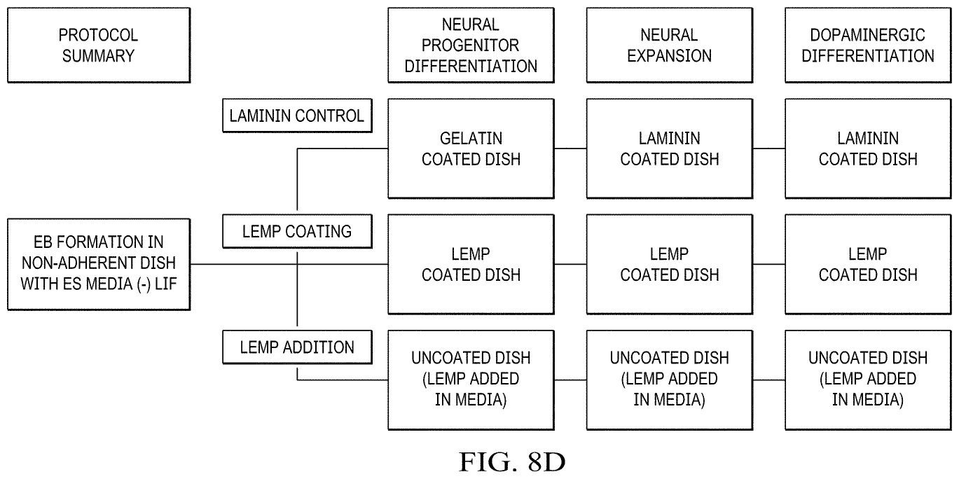

Extremely simple, well defined, and broadly applicable system for both 3D growth and differentiation of SCs. LEMPs have a precisely defined chemical formula, making it easy for use in GMP protocols. To use LEMPs no complicated steps are involved. LEMP is simply added to the culture dish/well after SCs and media have been added. The LEMP system works for all kinds of culture and differentiation media (at least for the ones the present inventors have tried so far including motor neurons: FIGS. 7A-B, dopaminergic neurons: FIGS. 8A-D, and cardiomyocytes. All of these differentiations are done in non-coated dishes, and no surface coatings (gelatin or laminin or matrigel) are required. Any working differentiation protocol can be easily adapted for use with LEMPs. In one embodiment this is done by foregoing the step that requires coating of dishes with materials such as laminin, and instead adding LEMPs to the culture media without any other change.

No agitation or shaking is required because 3D spheroids do not grow to very large sizes. The present inventors use static cell culture conditions and the present inventors have not observed formation of very large spheroids. Thus, shear forces due to excessive shaking are not required. Gentle and slow rocking could be incorporated to enhance nutrient uptake into spheroids during scale up.

Design and synthesis of LEMPs. Laminin and ELP motifs are shown in FIG. 2A. In order, the sequences have SEQ ID NOS: 1 to 5, specifically, the E₁₂ portion of elastin is SEQ ID NO: 6 (having 12 repeats), and the E₂₄ version is SEQ ID NO: 7 (having 24 repeats). The original backbones of pET-24a(+)-E₁₂ and E₂₄ were used from the previous published work³⁹. Custom oligonucleotides coding for laminin and VGKKKKKKKKG (SEQ ID NO:2) motifs were synthesized by Integrated DNA Technologies Inc. (IA, USA). These motif sequences were then inserted into the pET-24a(+)-E₁₂ and E₂₄ plasmid so that they would be translated at the N terminal of the LEMP. This was done according to the protocol from Chilkolti's lab⁴⁰. DNA sequence was confirmed after ligation (3130 Genetic analyzers, Applied Biosystems, Center for Biotechnology and Genomics, Texas Tech University, TX, USA). In addition to the laminin motif, the present inventors have also included a polylysine motif postulating that it might help in cell attachment of a broad cell type and their ability to guide neuronal outgrowth^{38,41}. This LEMP design might be of particular importance during in vivo transplantation because it could increase survival rates of transplanted dopaminergic or other neurons. LEMPs were purified based on thermal cycling of the impure LEMP protein mixture from 4° C. to 37° C. and back to 4° C. with a washing step in between. This cycling was done 6-8 times. Any residual endotoxins were removed as described before. To confirm the molecular weights of LEMPs, MALDI analysis and SDS PAGE gels were run as described earlier (data not shown). The transition temperature of these LEMPs were identified by taking 25 μM solutions of each LEMP and measuring the optical density (OD) at 350 nm as a function of temperature (Cary 300, Varian Instruments) (FIG. 2B). The data shows that LEMPs based on E₂₄ have Tₜ less than 37° C.

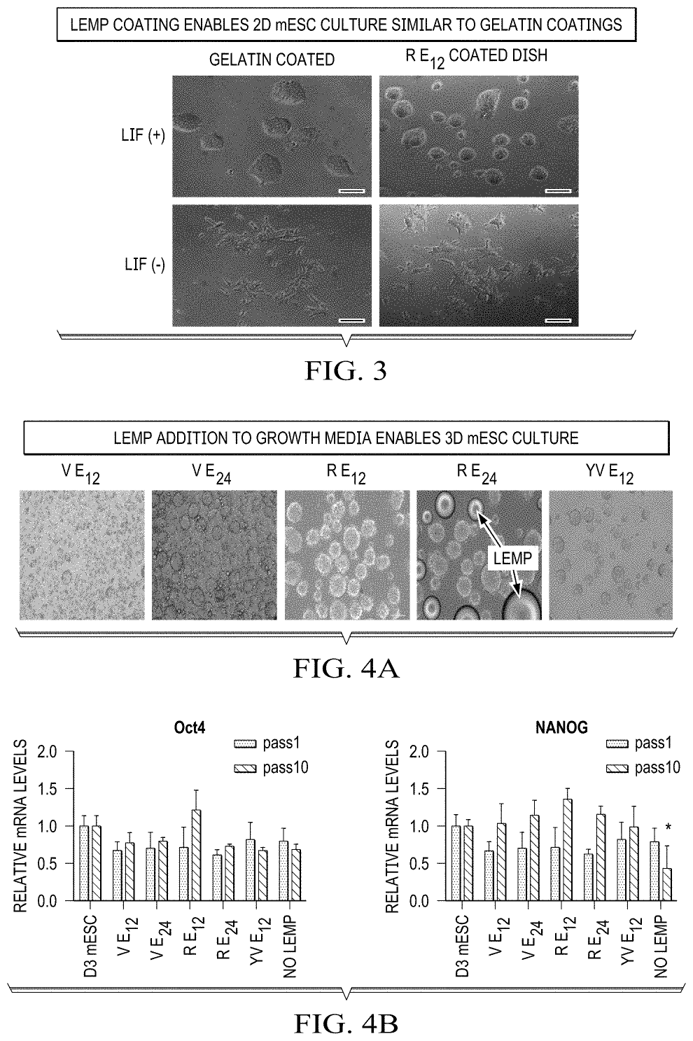

Coating of LEMPs on a culture dish leads to 2D SC culture similar to gelatin-coated surfaces. The present inventors first evaluated whether LEMPs can function as a cell culture support system in 2D by coating them on culture dishes. Different LEMPs were incubated for 1 h at 37° C. in the plates and washed with PBS also at 37° C. Next mESCs were added for culture either with or without leukemia inhibitory factor (LIF). As a control the commonly used approach of gelatin coated dish was used. After 3 days, the morphology of mESCs on LEMP-coated (R E₁₂ as representative example) and gelatin-coated dishes were similar in the presence of LIF (FIG. 3), demonstrating that LEMPs have the potential to help propagate ES cells. As expected, when LIF was not added, mESCs spontaneously differentiated for both LEMP- and gelatin-coated dishes.

Simple addition of LEMPs to the culture media leads to long-term 3D growth of mESCs. To test that suspended ECM-blocks in the form of LEMPs can support 3D cell growth the present inventors cultured D3 mESCs in the presence of different LEMPs in nonadherent dishes by simply adding the respective LEMPs into the culture media. Briefly, SC culture media with D3 mESCs (1×10⁵/ml) was added in to nonadherent dishes and LEMP (804) was then directly added into the culture dish. Cells were allowed to grow for 4 days and passaged by first washing with PBS at room temperature, treating with accutase at 37° C. to dissociate 3D spheroids into single cells, which were then passaged. A total of 10 passages were done, and at different passages, separate assays were done to confirm pluripotency of the cells being passaged.

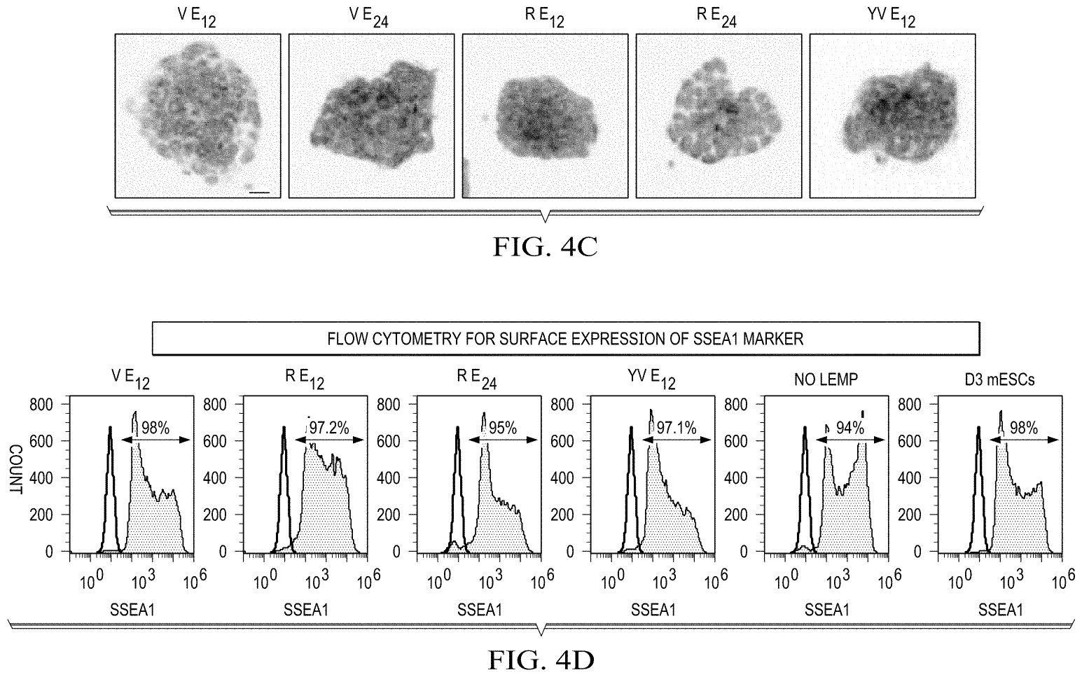

As seen in FIG. 4A, all LEMPs when added to the media helped to grow mESCs in 3D as a suspended mass of cells with a good spheroidal shape and a diameter ranging from 50 to 300 μm. However, for V E₁₂ the spheroids were small. It should be noted that V E₁₂ has the highest (52° C.) Tₜ amongst the LEMPs that the present inventors have created, and has a net positive charge as compared to other LEMPs, which could explain the small diameter of the spheroids formed. It is also important to note that R E₁₂ also has a high Tₜ of 49° C., but it was still able to induce formation of good-sized spheroids, suggesting that it is not just the Tₜ that is important, and thus more investigation is needed to understand the mechanism of how LEMPs sustain 3D culture of SCs. And this further investigation is part of the proposed Aims. To further confirm self-renewal capability, the present inventors performed quantitative real time polymerase chain reaction (qRT-PCR), immunocytotochemistry, and fluorescence-activated cell sorting (FACS) analysis at different passages from 1 to 10 examining the pluripotency markers. For this, at the step of single cell generation for passaging, part of the single cell suspensions were used for passaging and the remaining were used for analysis. Octamer-binding transcription factor 4 (Oct4) and Nanog self-renewal marker gene expression (qRT-PCR) was compared at passages 1 and 10 (FIG. 4B). Nanog gene expression was low at early passage #1 as compared to control D3 mESC grown on 2D surface, but as the cells adapted to the 3D culture at passage #10 this level increased to levels similar to control D3 mESCs grown on 2D surface. The exception was when no LEMP was added in which case a significant drop in Nanog gene expression was seen (p=0.004, FIG. 4B, right panel). Oct4 protein expression was reconfirmed by immunocytochemistry at passage #5 (FIG. 4C), while SSEA1 was confirmed using FACs (FIG. 4D). FIG. 4D shows that greater than 95% percent of the cells in each group were positive for SSEA1. Based on trypan blue staining greater than 95% live cells were seen. Further, mESCs from the 3D cultures were used to make embryoid bodies (EBs) using the conventional 4−/4+ retinoic acid protocol and then plated on to gelatin coated dishes, which led to the development of all three germ layers on day 14 (data not shown due to limited space).

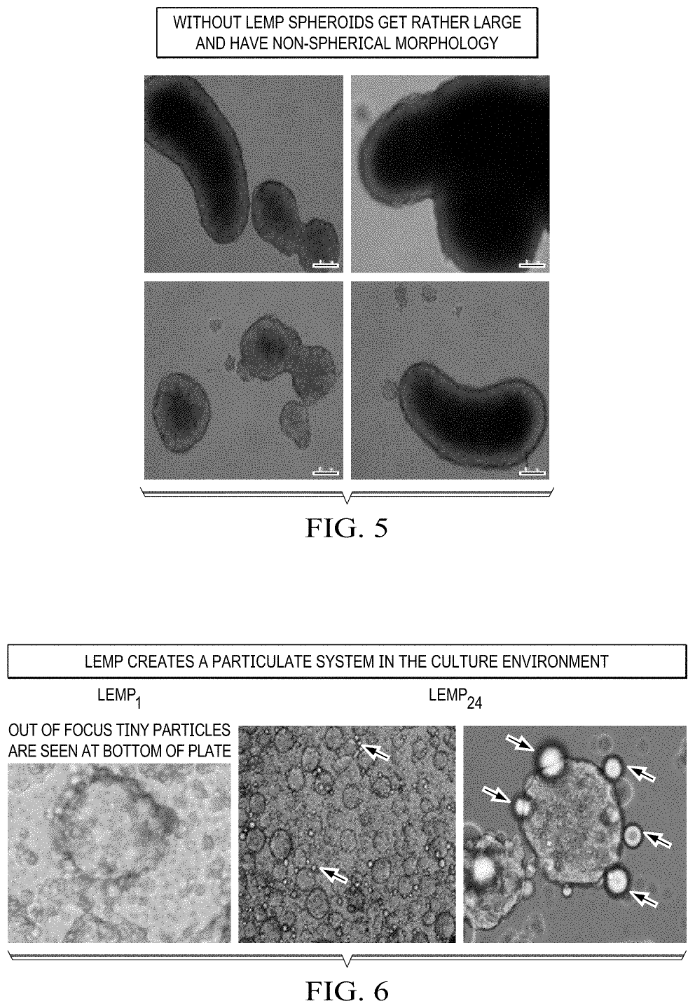

The importance of LEMPs is visually shown in FIG. 5, which shows that in the absence of any LEMP the morphology of 3D cells at late passage numbers is no longer spheroidal and they form large and irregular shaped bodies. Overall these experiments show that LEMPs when added to mESCs allow high number of passages while maintaining self-renewal capability without forming large spheroids.

Physical state of LEMPs on 3D spheroids. To get a better understanding of how LEMPs arrange themselves in the 3D culture system, the present inventors performed light microscopy imaging. As seen in FIG. 6 LEMPs (based on both E₁₂ and E₂₄ motifs called LEMP₁₂ and LEMP₂₄, respectively in the figure) can be seen to form particles that are widely distributed in the culture volume. The particles are however, larger in the LEMP₂₄-based system, likely due to lower Tₜ. Thus, LEMPs are able to form a suspension of ECM-blocks, which can interact with the 3D cell mass throughout the volume of the culture medium.

LEMPs help to differentiate mESCs into motor neurons. After demonstrating that LEMPs can be used to grow mESCs in 3D the present inventors proceeded to determine if they can also be used to differentiate SCs. The present inventors selected two protocols (i) motor neuron differentiation, and (ii) dopaminergic neuron. For the motor neuron differentiation the present inventors compared the conventional laminin-coated dish protocol as described before⁴³ with the LEMP-addition protocol. The present inventors used single cell suspension of mESCs in both protocols. Brief protocol details are given in the legend for FIGS. 7A-B. For the LEMP-addition protocol the same growth media conditions were used as for the laminin-coated protocol, with the notable differences that (i) the present inventors did not use laminin coated dishes but used non-coated dishes, and (ii) added the LEMP R-E₁₂ at two different concentrations (5 and 10 μM) into the media every two days at the time of media changes. On day 14 after neural differentiation, the present inventors analyzed neural protein and gene expression. Immunocytochemistry for the neuronal marker, Neuron-specific Class III β-tubulin (Tuj1), shows that in the LEMP protocol larger 3D like neuronal structures were formed as compared to the laminin-coating protocol (FIG. 7A). Furthermore, higher expression of Nestin (neural progenitor marker) and Tuj1 (neural marker) was seen in the LEMP protocol as compared to the control laminin group (qRT-PCR, FIG. 7B). A concentration dependent effect of R-E₁₂ was also seen. At a higher concentration of RE₁₂ the expression of Tuj1 was higher, while at a lower R-E₁₂ concentration the expression of Nestin-1 was higher. This demonstrates the ability of differentiating D3 mESCs into motor neurons by simply adding LEMP into the respective media without the use of laminin-coated dishes.

(6) LEMPs help to differentiate mESCs into dopaminergic (DA) neurons. To test if the LEMP system can be used in other differentiation protocols, the present inventors next proceeded to determine the potential of LEMPs to differentiate mESCs into dopaminergic neurons. For this the present inventors used a previously described⁴⁴ protocol. Briefly the mESCs were first induced in neural specification medium into midbrain-specified progenitor cells, which were then expanded, and then terminally differentiated into mature dopaminergic neurons in DA maturation medium. The entire differentiation workflow takes 30-35 days. The different groups were: (i) Control laminin-coating group, where laminin coated surfaces were used for differentiation; (ii) LEMP-coating group, where LEMP coated surfaces were used for differentiation; and (iii) LEMP-addition group, where uncoated surfaces were used for differentiation but LEMP was added into the culture/differentiation medium every time media was changed. Dopaminergic neurons were characterized by qRT-PCR and immunocytochemistry. Based on qRT-PCR gene expression analysis there was no difference in neural marker (Tuj1, FIG. 8A) expression between the LEMP groups (both coating and adding) versus the control group (laminin coated dish). However, midbrain dopaminergic marker, Tyrosine Hydroxylase (TH), FIG. 8B) expression showed slight dependence (not statistically significant) on treatment groups, and YV E₁₂ showed slight decrease. However, when R E₁₂ was mixed with YV E₁₂ the gene expression was seen to increase. Although these are qualitative trends, this does suggests that some synergy might be expected by mixing different LEMPs. Immunocytochemistry (FIG. 8C) demonstrated that for every group, the protein expression of Tuj1 and TH was similar. Thus, these results shows that the simple addition of the LEMPs of the present invention has the same effect as the more cumbersome laminin-coating approach for differentiation of mESCs into dopaminergic neurons. FIG. 8D: Protocol Details: The present inventors followed the method described previously⁴⁴. Briefly, embryoid body (EB) was formed. On the 4th day, the EBs were collected, dissociated, and either (i) plated on 0.1% gelatin-coated dishes (control), or (ii) plated on LEMP-coated dishes (10 μM, 37° C., 1h), or (iii) added to uncoated dishes without any coating but with LEMPs (5 μM) LEMP-addition groups. To initiate neural differentiation, cells were cultured in DMEM/F-12 media containing neural N2 supplement for 7-9 days with media replacement every 1-2 days. For the LEMP-addition group fresh LEMP was added during these media changes. Next, cells were detached from plates of control (gelatin-coated) and LEMP-coated groups and plated onto a dish coated with laminin (for control group) or respective LEMP (for LEMP-coated group) at a density of 75,000 cells per cm2. For LEMP-addition groups the cells were continually cultured in the same dish without dissociation. After 24 hours, these neural cells were expanded further by changing to the DMEM/F12 medium supplemented with B27 supplement and several other factors such as bFGF, Sonic hedgehog, basic fibroblast growth factor 8b for 4 days. Terminal differentiation into dopaminergic neurons was performed by culturing these expanded neural cells in neuronal-expansion media (DMEM/F12 media containing ascorbic acid instead of bFGF) for 8-10 days. After 20 days of terminal differentiation, the present inventors performed analysis with qRT-PCR and Immunocytochemistry.

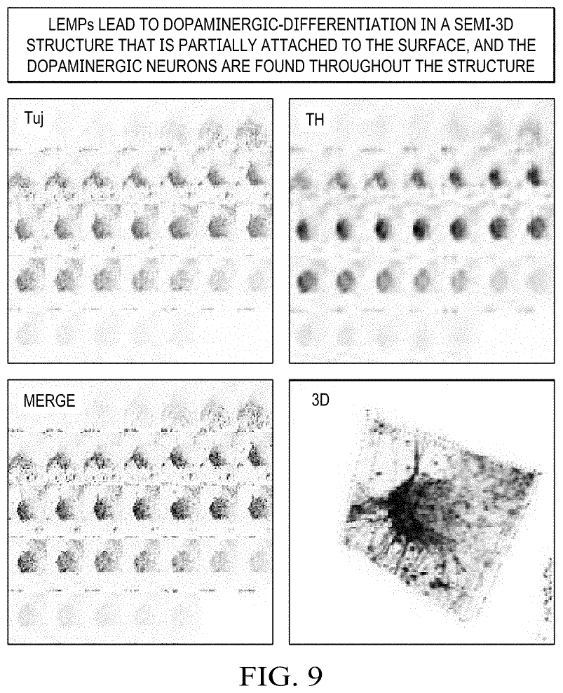

The present inventors also noticed that with LEMP-based differentiation, many nodules that were attached to the plate were formed. These nodules were larger and more in number in the LEMP protocol versus the laminin-coated protocol. The present inventors immunostained these nodules for Tuj1 and TH, and performed confocal sectioning. The present inventors found that the Tuj1 and TH was localized even in the interior of the nodules (FIG. 9). This suggests that LEMPs can allow for a more 3D-like differentiation rather than simply 2D planar differentiation.

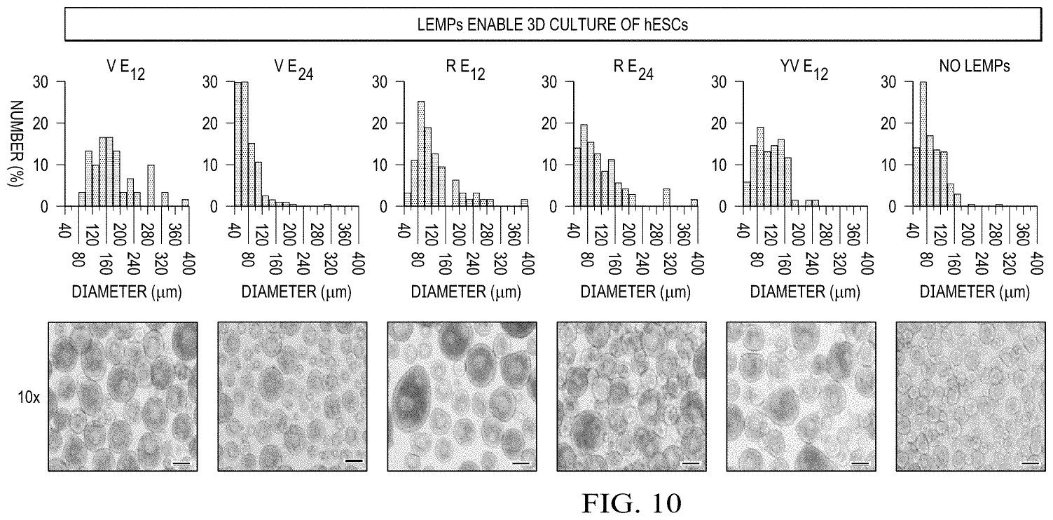

Human ES cells can be grown in 3D cultures in the presence of LEMPs The present inventors next evaluated the ability of LEMPs to grow 3D cultures of human ESCs. Thus, the present inventors used the H9 human ES cell line and followed the same approach of culture as the present inventors had followed for D3 mouse ESCs. Briefly, single cell suspensions of hESCs were made and plated in nonadherent dishes, into which different LEMPs were added at a concentration of 8 μM, and the cells were cultured for 4 days in static culture. hECS morphology was checked at passage #2 and diameter of the 3D spheroids was measured. FIG. 10 shows that LEMP treated cells in general have larger spheroid diameters as compared to cells not treated with LEMP, and all groups show general ES-like morphology demonstrating that treatment with LEMP does not negatively affect the human ESCs. At the time of writing this grant the present inventors are still continuing the passages. As seen in mESCs, the present inventors expect that at longer passages, when no LEMP is added, the hESCs become irregular in shape as did mESCs (FIG. 5).

Summary of the studies with the LEMP of the present invention. These data shows that the present inventors have engineered a novel biomaterial, which the present inventors call LEMP, and the present inventors have successfully applied it towards 3D SC culture and differentiation of mouse ESCs. LEMPs have shown the ability to readily substitute steps involving laminin-coated dishes for both motor and dopaminergic neuronal differentiation protocols. The LEMP protocol is extremely convenient and easy to implement. It involves non-adherent dishes and LEMP is simply added to the growth or the differentiation media containing the ESCs; and the culture is allowed to continue until media change is required, at which point LEMP is again added along with the fresh media. This approach has also been successful with hESCs as an example of cells.

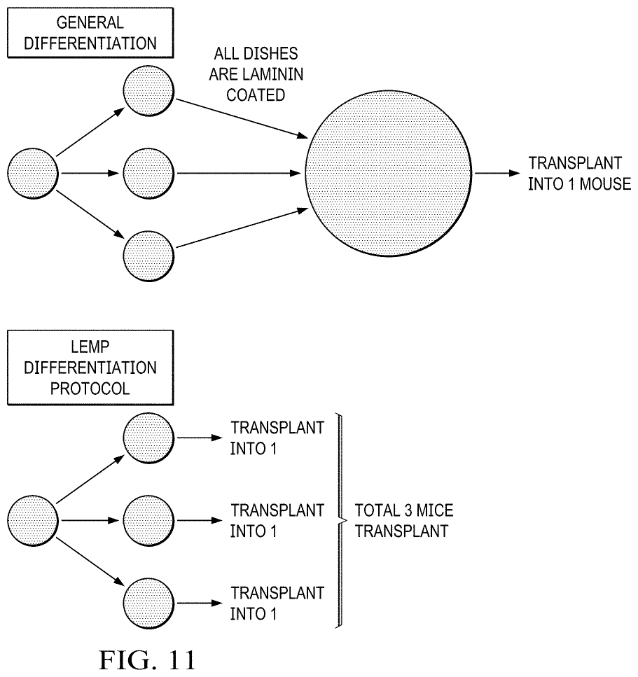

FIG. 11 shows a comparison of a ‘general’ protocol of the prior art (top), compared to the ‘LEMP’ protocol for differentiation of the present invention (bottom). The advantages of the present invention over the general protocol of the prior art include: (1) more cells obtained from same starting cell number, (2) more subjects can be treated; (3) cells are available sooner for transplantation; (4) cost saving because laminin coatings are not required; (5) cells grow in 3D state and differentiate in 3D like state; (6) saving time by not coating dishes with laminin and faster differentiation, which cuts final time to about 14-16 days; and/or (7) Laminin coating has variability, which is eliminated through LEMP protocol. The traditional method of cardiomyocyte generation requires a complex process and many growth factors.

### EXAMPLE 2

### Characterization of ELP

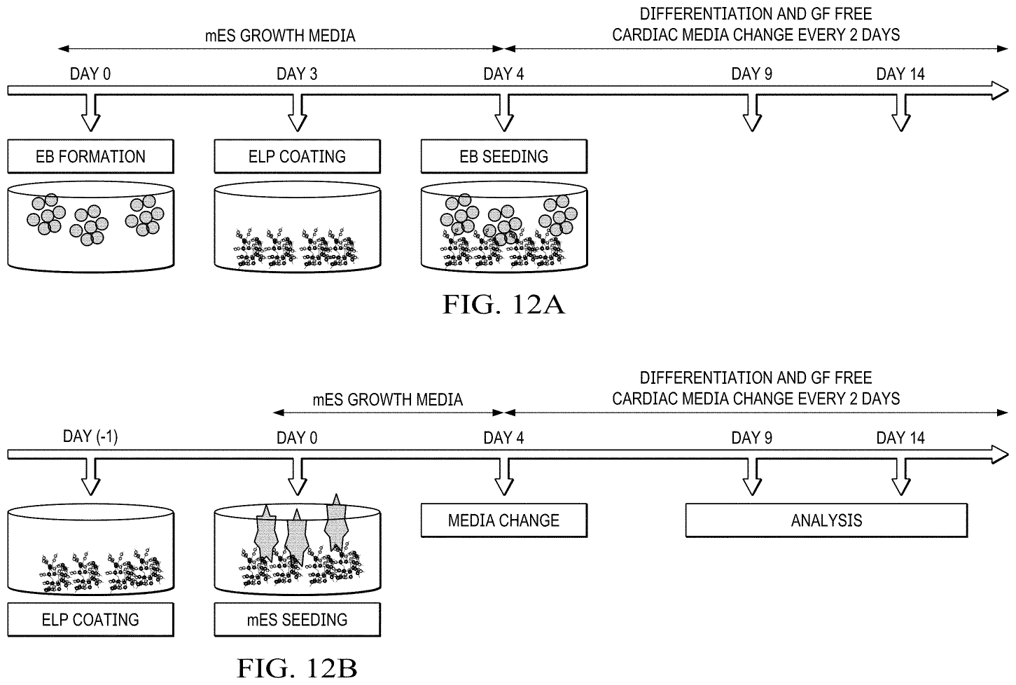

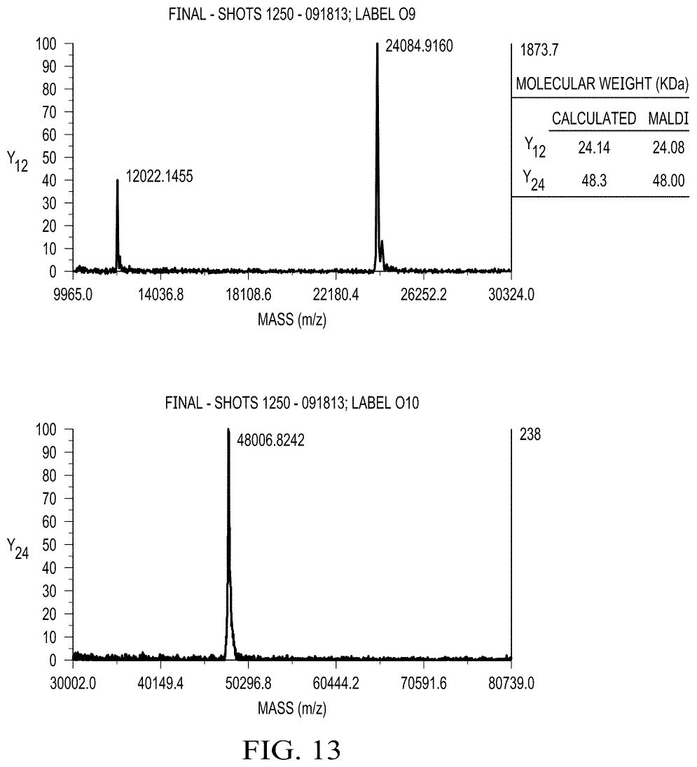

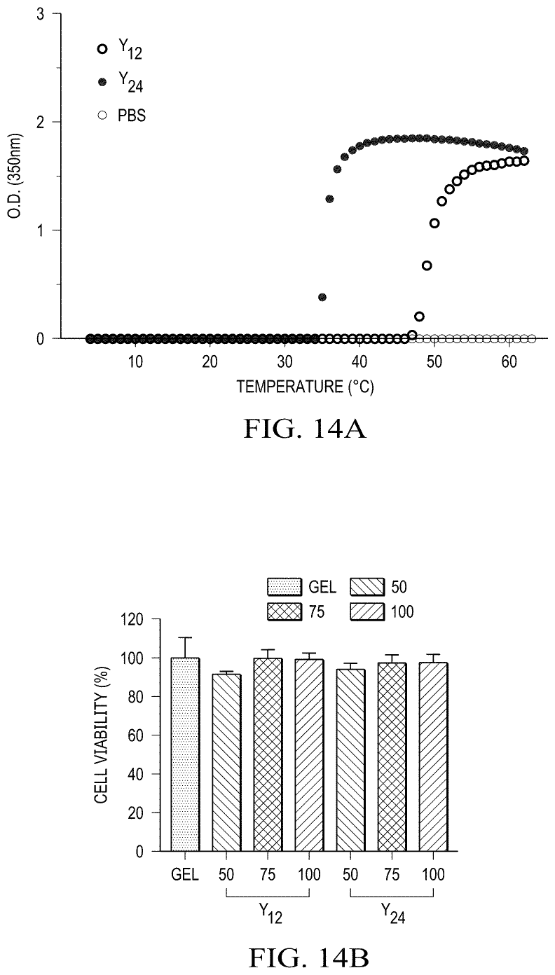

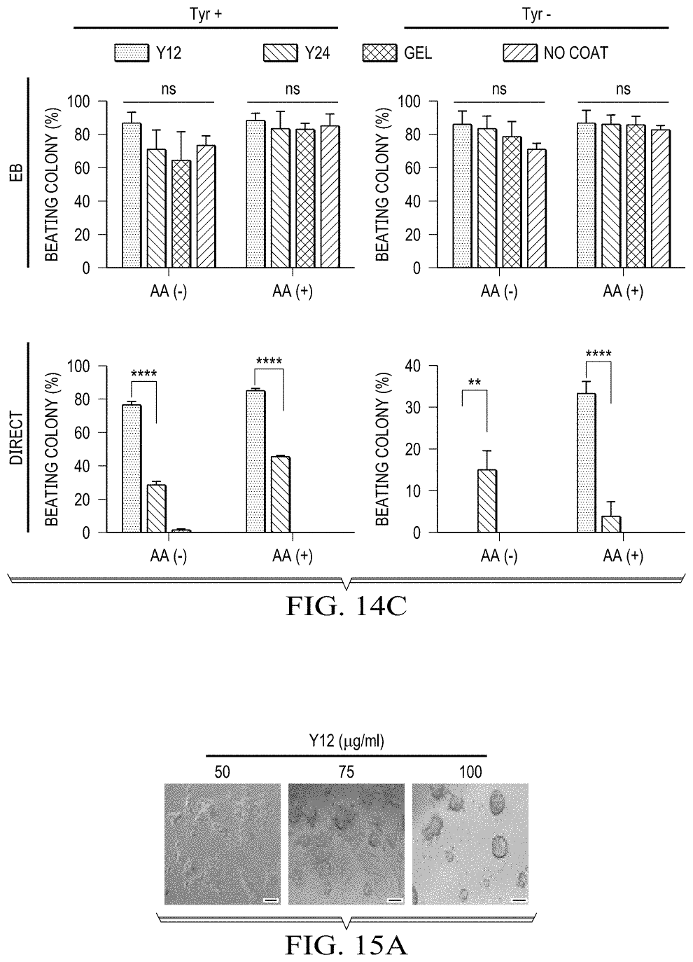

Characterization of ELP. For in vitro cardiac differentiation of the mESCs, the inventors used both embryonic body and direct differentiation method based on several protocol with some modifications (FIGS. 12A and 12B). The inventors combined tyrosine and alanine to make the hydrophobic ELP sequence and the repeat block is Y₁₂ or Y₂₄=[(GAGVP)₂(GYGVP)(GAGVP)₂]_{12 or 24} where 12 or 24=number of repeats of ELP monomers. The ELPs were confirmed by analyzing their molecular weights using MALDI-TOF (FIG. 13). The Tt of these ELPs was confirmed by taking a 25 μM solution of each ELP and measuring the optical density (OD) at 350 nm using a UV-vis spectrophotometer (FIG. 14A; Cary 300, Varian Instruments). The data shows that Tt of Y₁₂ ELP was in range of 48° C. Changes in hydrophobic amino acids such as tyrosine and alanine in the ELP pentapeptide repeat at Y₂₄ further reduced the Tt to 38.67° C. MALDI and SDS PAGE gels analysis were performed as described in previous report [42] to determine the molecular weight of the ELP (FIG. 13). To compare the effect of ELP in inducing cardiac differentiation of D3 mESCs, various conditions depending on presence or absence of tyrosine crosslinker and cardiac differentiation factor or growth factor were performed.

Cell viability of cross-linked Y₁₂ ELP. In order to investigate the cytotoxicity of crosslinked Y₁₂ and Y₂₄ ELP, cell viability was tested after growing D3 mESCs for 2 days at concentrations of crosslinked 50, 75 and 100 μg/ml. As shown in FIG. 14B, Y₁₂ ELP and Y₂₄ ELP showed similar cell viability as the control at all concentrations. First, the inventors did not observe significant changes in cell viability at various concentrations of ELP, and overall, 75 μg/ml of ELP concentrations were the most optimal. Therefore, all the experiments were carried out at this concentration.

Determination of differentiation method for cardiomyocytes. For cardiac differentiation, the inventors examined the possibility of using EB formation methods using 75 μg/ml of Y₁₂ and Y₂₄ ELP cross-linked coated dish. respectively. After 4 days of EB formation, the inventors seeded the EB to the each cross-linked ELP coated dish and then checked the beating rate at 9 days. A 0.1% gelatin coated dish was used as a control. EB began to adhere for 2-3 hours after seeding and was fully attached to the dish within 24 hours. By day 6, the EB was fully enlarged in an outwardly extended flat shape and the middle portion increased slightly. After 7 days of cardiomyocyte differentiation, beating EB began to appear, and most EB began to spontaneously beat at different sizes.

To investigate the effect of AA to induce cardiac differentiation of stem cells, the inventors compared the cardiomyocytes differentiation efficiency in ELP coated dishes with AA (FIG. 14C). As expected, AA treated group showed a generally better beating rate than the AA-free group, but there was no statistically significant difference between groups.

Direct differentiation methods showed a beating colony ratio close to 85% in crosslinked Y₁₂ ELP coated plates treated with AA and less than 70% in non-AA treated controls. In particular, Y₂₄ showed lower overall differentiation rate than Y₁₂ regardless of AA treatment (FIG. 14C). Finally, Y₁₂ ELP showed the maximum beating colony rate in all EB and direct differentiation methods. In contrast, only a few beating colonies were observed in the control study using gelatin-coated dishes.

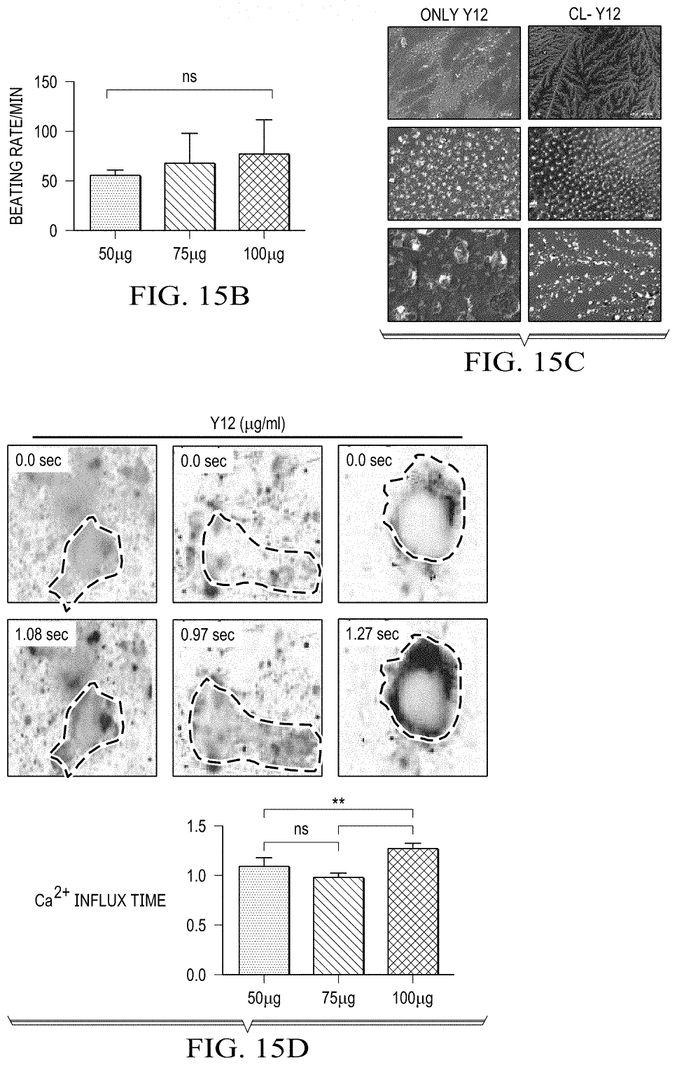

Next, the inventors tested the effects of Y₁₂ ELP on cardiomyocyte differentiation of mESCs. After cross-linking Y₁₂ ELP for 2 days at each concentration, the ES morphology showed a slight monolayer formation at 50 μg/ml, whereas at 75 μg/ml, many colony morphologies similar to the 3-dimensional structure were observed (FIG. 15A). On the 9th day after seeding, a total beating colony was counted. Most of the beating frequencies were between 70 and 90 times/min in each concentration of ELP (FIG. 15B). SEM analysis was performed to compare surface differences of the culture dishes after cross-linking coating. As shown in FIG. 15C, it was observed that the crosslinked Y₁₂ ELP was coated with a more uniform size pattern of particles than the non-crosslinked groups.

Ca²⁺ influx of cardiomyocytes in cross-linked ELP coated dish. To characterize the occurrence of Ca²⁺ during cardiomyocyte beating, the inventors used a fluorescent intracellular calcium sensor, Fluo-4, during cardiomyocytes beating. Confocal line-scan recordings were performed in 50, 75 and 100 μg/ml concentration Y₁₂ ELP coated dish. FIG. 15D shows an image taken from a low-speed video that captures calcium influx during three-dimensional shrinkage. Average values of time-to-peak of the distribution of cardiomyocytes during 30 second were calculated at 50, 75 and 100 μg/ml. Fast peak of cardiomyocytes was present in both groups (50, 75 μg/ml), depending on the duration of the peak. However, the duration of the shrinkage peak was significantly longer as the concentration of ELP became higher.

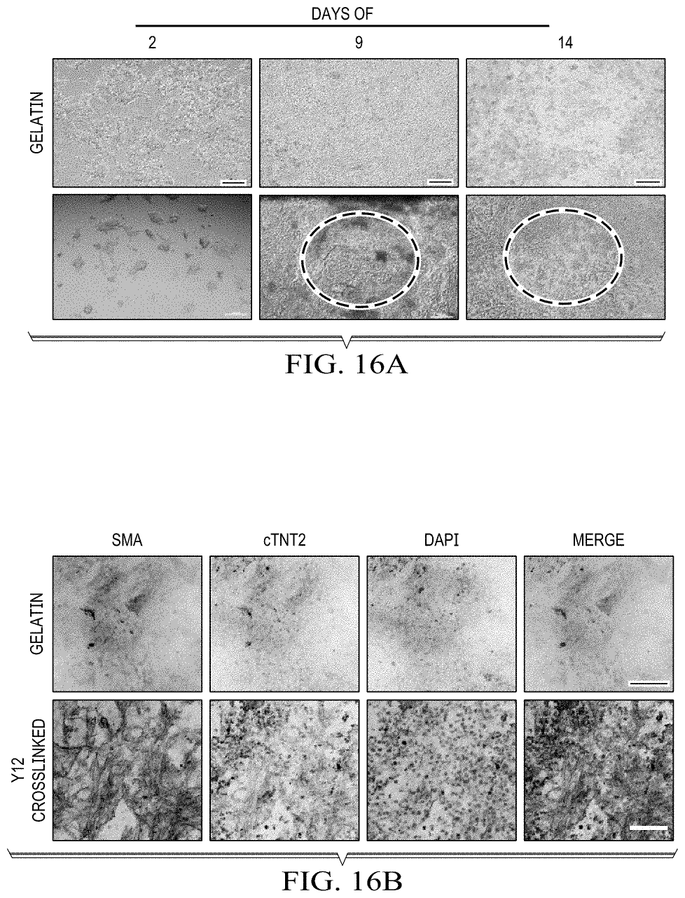

Immunostaining of cardiomyocytes grown in cross-linked Y₁₂ ELP. After D3 mESCs were induced to differentiate into cardiomyocytes in a cross-linked Y₁₂ ELP coated dish without AA, they were stained for cardiomyocytes-specific marker cardiac troponin t (cTNT2) and smooth muscle actin (SMA). Cell nuclei were marked blue by DAPI staining. Before immunostaining of cardiomyocytes, experimental time was set to day 2, 9 and d 14, and cell morphology was observed compared to control (gelatin coated dish, FIG. 16A). Double immunofluorescent staining for cTNT2 and SMA is shown in FIG. 16B). In the crosslinked Y₁₂ ELP coated dish, the differentiated cells showed more positive staining of cTNT2 and SMA than the gelatin coated dish group. SMA immunostaining showed that actin was organized into filaments in mostly stained cells. These results suggest that the crosslinked Y₁₂ ELP improves cardiac differentiation of mESCs.

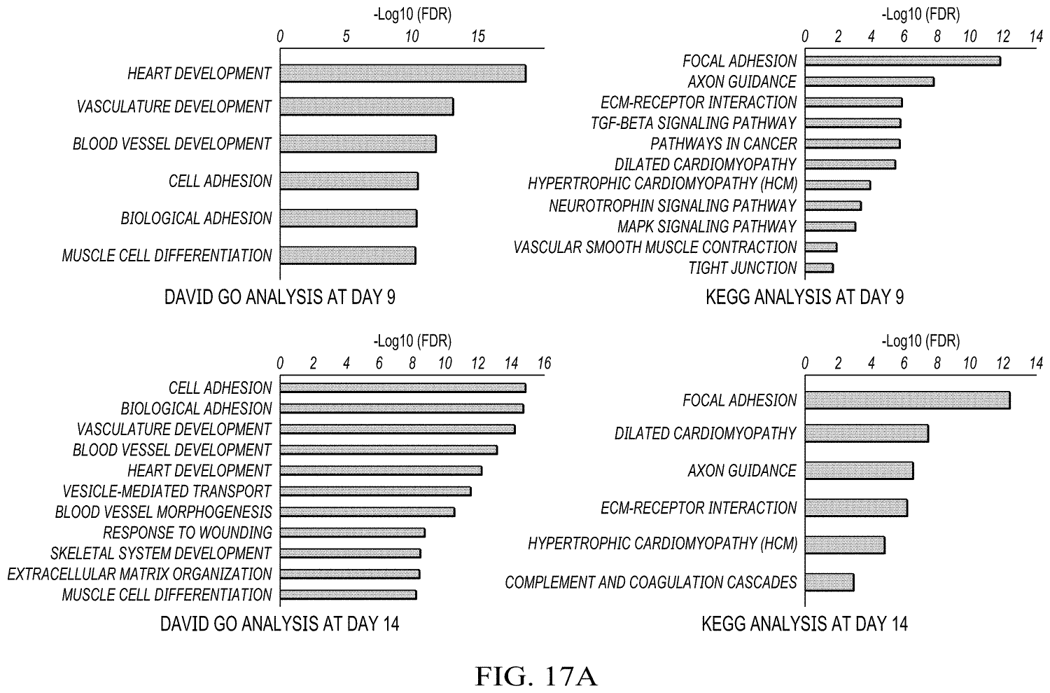

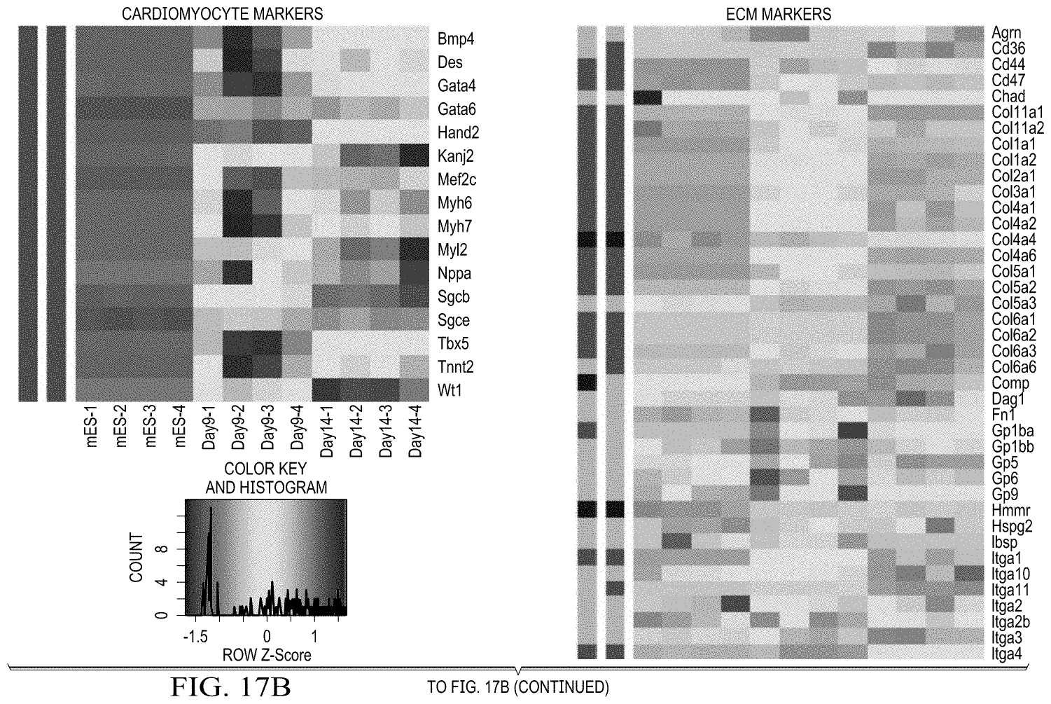

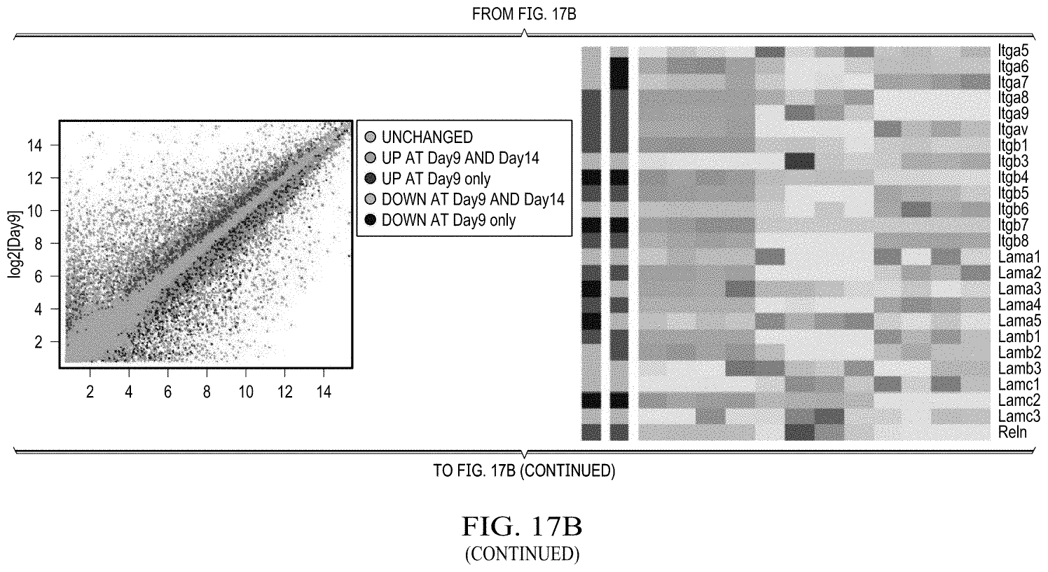

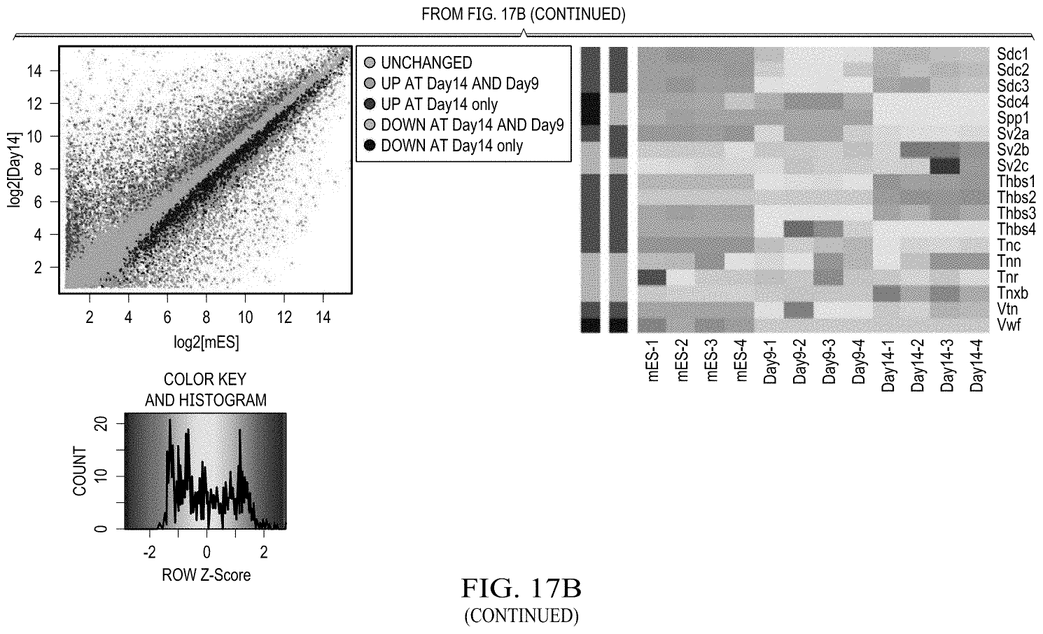

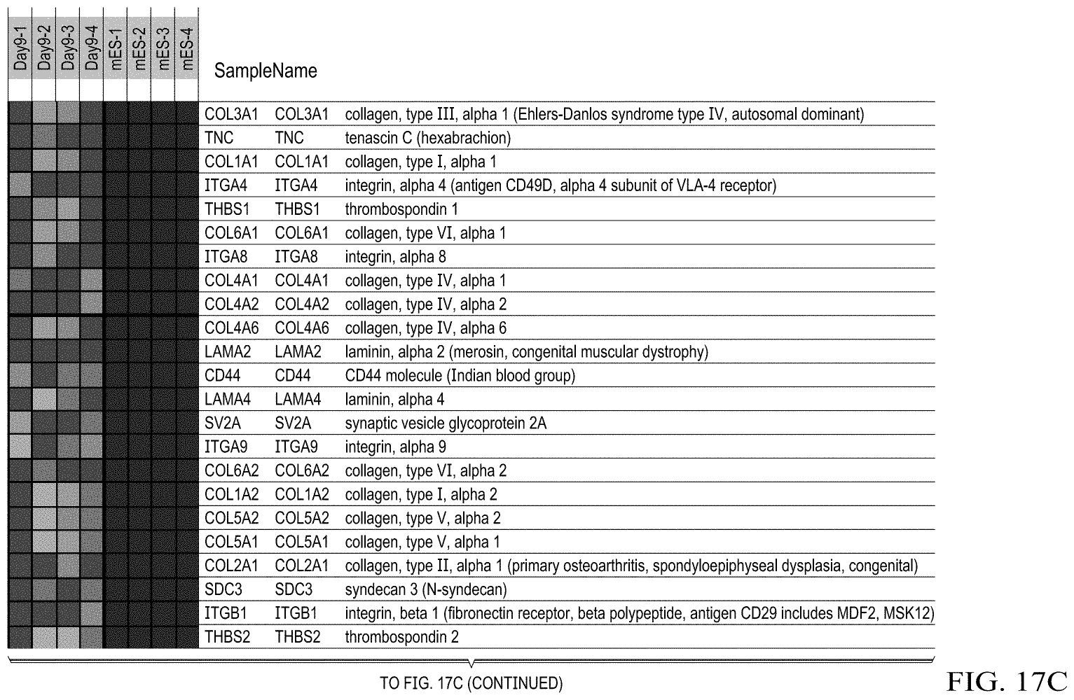

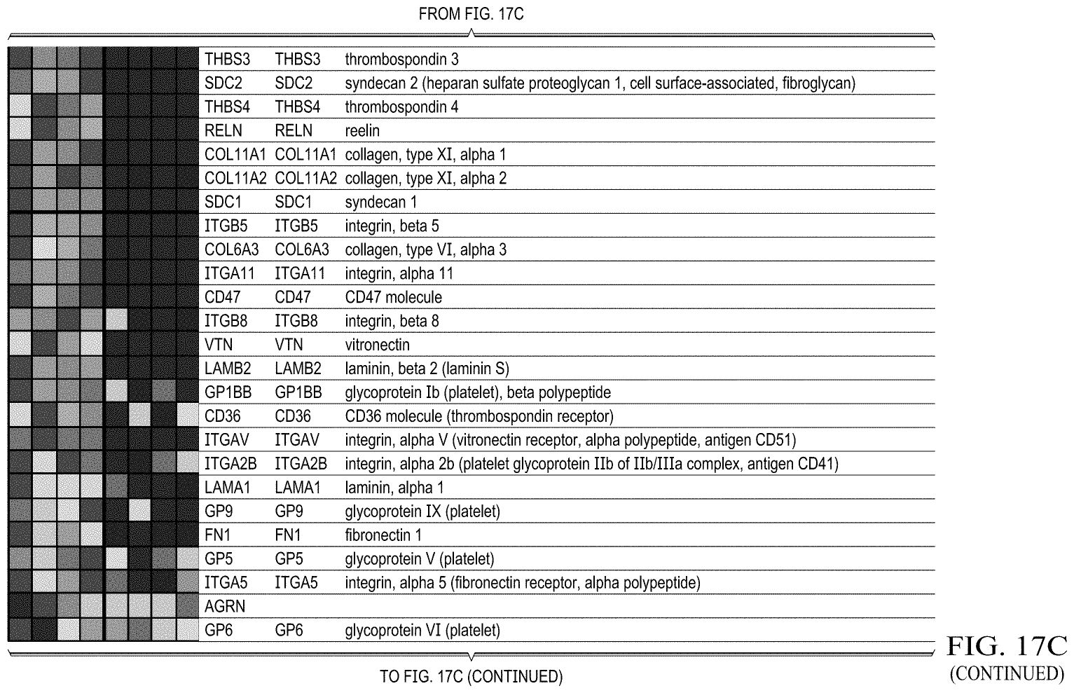

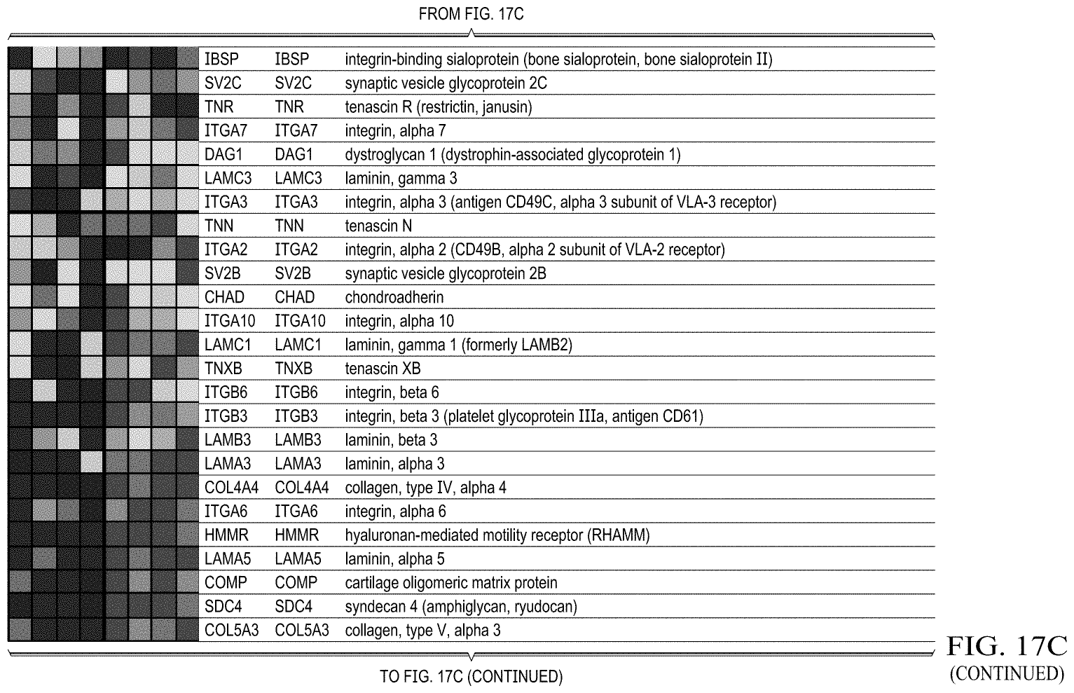

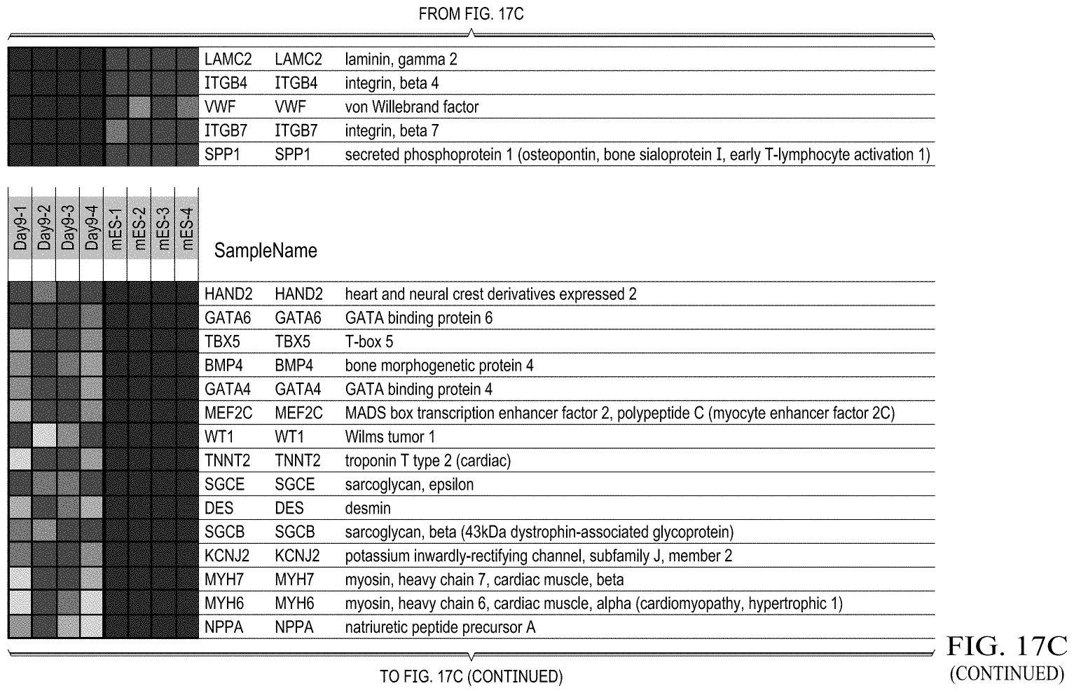

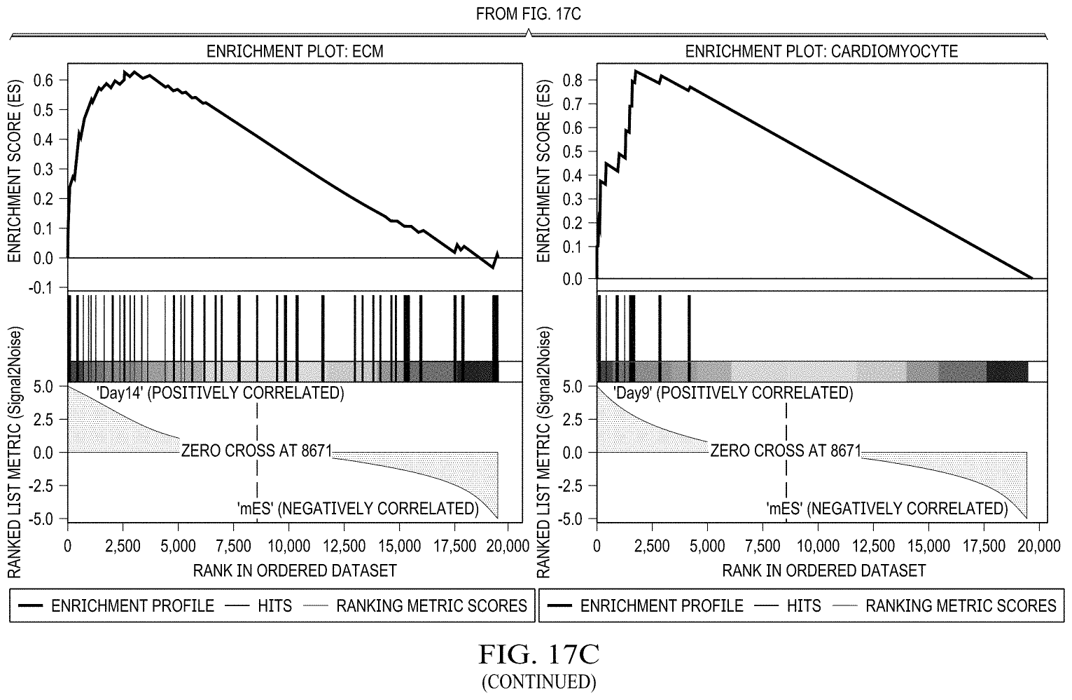

Microarray. For analysis of global transcriptome of cardiomyocyte which were grown in cross-linked ELP, the inventors conducted the microarray is shown in FIGS. 17A to 17C. First, the inventors profiled RNA sample generated from undifferentiated mESCs, day 9 and day 14 after differentiation in crosslinked Y12 ELP coated dishes.

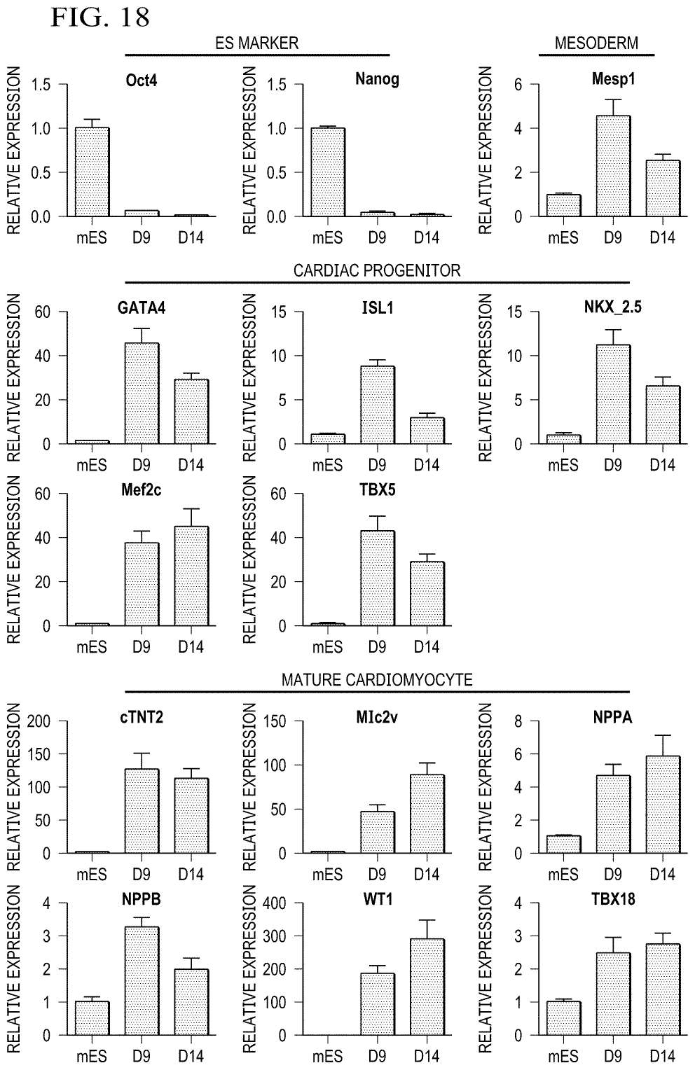

Microarray data validation. Differential regulation of specific gene transcripts was analyzed by qRT-PCR to verify microarray results. This is the universal gene (OCT4), ectoderm (TUj1), and intracardiac mesoderm (MESP1, MEF2C, GATA4, TBX5, NKX2.5 and CTNT2) along with the testimonies that represent the posterior machinery. The results are consistent with microarray data (FIG. 18).

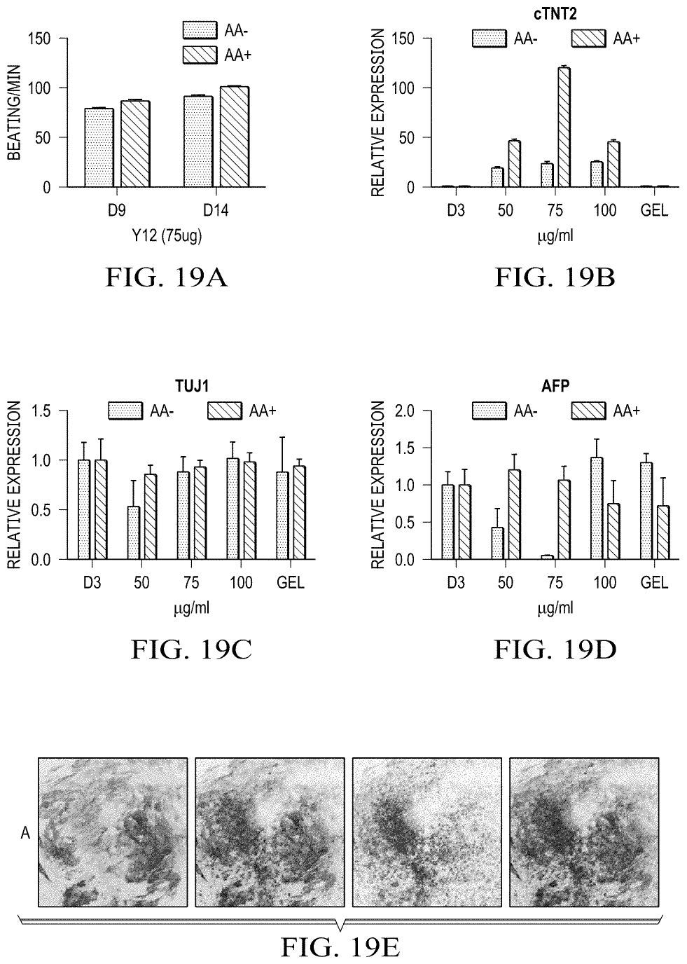

Effect of AA on direct differentiation of mES cells into cardiac myocytes. To investigate the effect of AA as one of the cardiogenic inducers using the crosslinked Y₁₂ ELP coated dish taught herein on direct cardiomyocytes differentiation, cells were treated with AA from 200 μM for 12 days from day 2 of differentiation. The beating rate gradually increased from day 9 until day 14, but the rate AA-treated group was slightly higher than the untreated group in the crosslinked Y₁₂ELP coated dish environment (FIG. 19A). Co-operatively, expression of the major cardiac gene expression of cTNT2 was strongly increased in the AA-treated group (FIG. 19B). However, the specific maker genes of ectoderm (Tuj1) and endoderm (AFP) lineage were very low in each group (FIGS. 19C, 19D). Also, it was confirmed that cTnT2 positive cardiomyocytes was strongly expressed in AA-treated group at day 15 by immunocytochemistry (FIG. 19E).

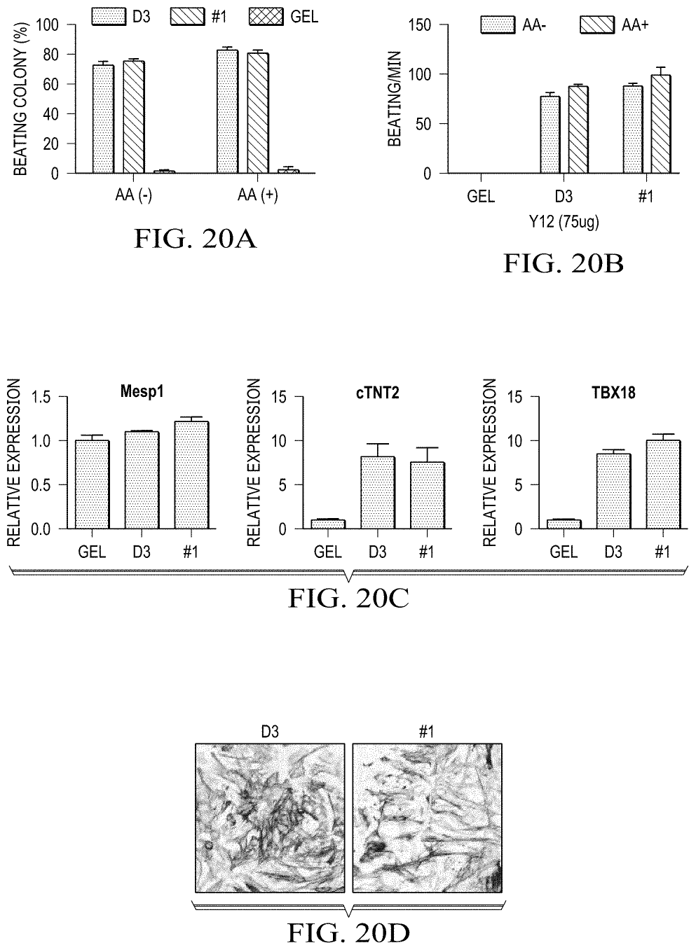

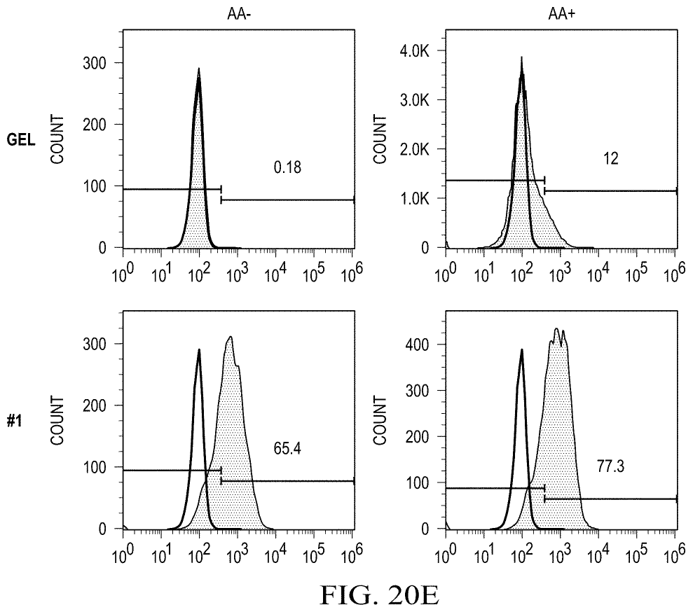

Cardiac differentiation of iPS cells. To demonstrate that the invention described herein can help in differentiation of not just embryonic stem cells but also induced pluripotent stem cells, the inventors used the miPSC line (IPS#1), which they produced using an ELP-based gene delivery system. The IPS#1 was used to determine the effect of cross-linked Y₁₂ ELP and to analyze the mechanism of promoting myocardial differentiation. It was seen that both the embryonic D3 and induced pluripotent stem cell line IPS#1 differentiated in cross-linked ELP into cardiomyocyte, and showed similar differentiation yields and beating rates, and the AA-added group showed a higher efficiency than the group without AA (FIGS. 20A-B). Also, IPS#1 showed gene expression rates higher than D3 mESCs when the expression of each gene was examined at each developmental stage even in the absence of AA (FIG. 20C). When the expression of CTNT2 of D3 and iPS#1 differentiated in cross-linked ELP was confirmed by immunostaining, both cells were found to strongly express it (FIG. 20D). Flow cytometric analysis of cardiomyocytes derived from miPSC#1 was performed using cTnT2 as a cardiac specific marker on day 14 post-differentiation to determine the differentiation rate of cardiomyocytes on a cross-linked Y₁₂ ELP coated dish. It was confirmed that iPSC line #1 significantly improved cardiomyocytes differentiation in the crosslinked Y₁₂ ELP dishes upon induction with AA than without AA. (FIG. 20E) FACS analysis of cTnT2 expression in D3 and IPS#1 cells differentiated in cross-linked Y₁₂ ELP coated dishes.

In this invention, the direct monolayer protocol using ELP that minimalized the cell damage by trypsinization and without the EB formation step on the differentiation of stem cells into cardiomyocytes was investigated. Through the differentiation pathway of cardiomyocytes and through the external environment coated with crosslinked ELP of mESCs, the inventors identified protocol(s) that ES cells differentiated into cardiomyocytes within 2 weeks of onset.

To date, many research groups have published a number of protocols to differentiate ES cells into cells like cardiomyocytes. However, a group of purely differentiated cells that have been removed from the culture medium for the factors necessary for differentiation into specific cells has not yet been reported. Although several protocols using protocols that depend on EB-forming methods have shown high cardiomyocyte yields for cardiomyocytes production in mESCs, the inventors often observe that yield varies between batches. In addition, this technique has a problem in that the growth factors required for differentiation of stem cells do not equally affect cells deep within the EB, resulting in a significant change in efficiency.

For this reason, the inventors first developed an EB-independent and differentiated monolayer protocol without cardiomyocyte differentiation factors such as BMP, Noggin, Activin, and ascorbic acid. Some groups have studied that spontaneously beating cardiomyocytes derived from adipose tissue-derived stromal vascular fraction using gelatin hydrogels. These cells showed the similar character to those of naive cardiomyocytes aspect of gene expression of CM marker, beating mode, Calcium activities and cTNT3 protein expression, but the rate of cardiomyocytes was very low (14.29%) [43-45].

Although spontaneous differentiation occurs in the culture medium without the necessary factors for cardiomyocytes differentiation, purely differentiated populations of interest have not yet been reported. This system developed by the inventors showed that mESCs and miPSCs could be induced by cross-linked ELP into cardiomyocyte. In addition, the ELP-based differentiation method taught herein proved that cells are able to differentiate into a cardiovascular lineage in a growth-factor-free environment.

The ELP based system disclosed herein included a much simplified method, and in vitro culture conditions stem cells can naturally differentiate into myocardial cells. Therefore, it is possible to try a new method for improving the efficiency of induction including use of other inducing agents, culturing of myocardial cells and delivery of a specific gene. Research by Takahashi [5] has shown that AA markedly increases the number of mESCs that undergo differentiation into cardiomyocytes in the absence of EB formation. AA is most commonly used because it has been reported that stem cells increase myocardial cells during myocardial differentiation. Therefore, the system showed high differentiation efficiency as a result of verifying high myocardial differentiation rate through combination of myocardial differentiation inducer such as AA.

In order to evaluate myocardial cell function, the inventors examined the shrinkage of each concentration. Despite delayed cardiomyocyte differentiation in the high cross-linking group (100 μg/ml), no significant difference in the rate of beating was observed between all the groups (FIG. 15B). The number of beating rate on the ELP-crosslinked dishes drastically increased on day 9-10 day of culture (FIG. 19B). This function of Y₁₂ ELP to enhance cardiac differentiation can be strongly supported by a significant increase in cTNT2 expression.

As Sterk reports that cationic polymer-coated surfaces enhance myocardial cell contraction, these results show that the ELP taught herein plays a similar role in improving cardiac differentiation and pulsatile induction [46]. A possible mechanism associated with cardiac cell function, but not a limitation of the present invention, may be due to an unknown interaction between Y₁₂ ELP and integrin.

The ELP-based monolayer differentiation method of the present invention shows that high yields can be obtained within 2 weeks after in vitro culture compared to other protocols. This shows that a single-layered platform of cell differentiation based on cross-linking ELP is superior to EB formation because all cells are exposed to equally cross-linked ELP and cardiomyocyte induction is achieved.

In addition, the cross-linked ELP-based monolayer culture method taught herein reduces cell stress by trypsin treatment and is very unlikely to adversely affect stem cell differentiation and viability. Indeed, it has been reported that cells that have undergone a monolayer differentiation protocol provide cells with increased survival rates after transplantation.

Prior to the present invention, the production of fully mature adult cardiomyocytes in vitro was still a major stumbling block. To solve this problem, the new cross-linked ELP-based differentiation method taught herein, including the combination with several growth factors provides a new method and cells for myocardial cell therapy.

A novel approach to induce cardiomyocytes using stem cells with cross-linked ELP is taught herein. The cross-linked ELP system demonstrated that immunofluorescent staining of proteins and mRNA expression levels of cardiac markers, cytoplasmic calcium transient activity and spontaneously pulsating myocardial cell-like cells could be derived from ES cells.

### EXAMPLE 3

### Crosslinked Elastin-Like Polypeptides Mediate Direct Cardiac Differentiation of Embryonic and Induced Pluripotent Stem Cells

Stem cell-derived cardiomyocytes have significant potential in the field of regenerative cell therapy for cardiovascular diseases. However, low purity and maturity of the generated cardiomyocytes is a bottleneck. This example shows the effect of elastin-like polypeptides (ELPs) on cardiac differentiation of mouse embryonic stem cells (mESCs), mouse induced pluripotent stem cells (miPSCs) and human embryonic stem cells (hESCs). ELPs were coated on culture dishes through enzymatic crosslinking, over which, the mESCs, miPSCs and hESCs were cultured. From these cultures, cardiomyocytes could be obtained with high expression of maturity markers, which were confirmed through immunofluorescent staining of proteins and mRNA expression levels. Cytoplasmic calcium transient activity verified spontaneously pulsating myocardial-like cells. When ascorbic acid (AA) was added during differentiation phase as a cardiac growth factor, the yield of cardiomyocytes further increased in mESCs and miPSCs. Microarray analysis indicated that ELP coating promotes differentiation of mESCs into cardiomyocytes likely through extracellular matrix signaling pathway interactions. These results demonstrate that crosslinked ELPs is an effective tool to induce differentiation of mESCs, iPSCs and hESCs into cardiomyocytes and as such, provide a useful technique for generating cardiomyocytes for regenerative medicine and tissue engineering applications.

The inventors investigated the effect of ELPs on cardiac differentiation of mESCs, miPSCs and hESCs. To the best of our knowledge, this evaluation has not been reported before. ELP at two different molecular weights were recombinantly synthesized to tune the Tₜ and investigated how the ELPs modulate cardiac differentiation using the monolayer direct differentiation method. The ELPs were coated on the surface of the tissue culture dishes, and subsequently mESCs, miPSCs and hESCs were cultured and differentiated on these coatings. By using crosslinked ELP coatings, it is shown herein that mESCs, miPSCs and hESCs can be induced to differentiate into cardiomyocytes with high expression of maturation markers, high purity levels, and similar beating rates as the cardiomyocytes obtained from differentiation using Matrigel. Therefore, these finding show that ELPs are suitable substrates for supporting differentiation of different stem cells including hESCs, mESCs, and miPSCs into cardiomyocytes, and can also be more broadly in the field of stem cell based regenerative therapy.

Expression, purification and protein expression of the ELPs. The ELPs were recombinantly synthesized using Recursive Directional Ligation by the Plasmid Reconstruction (PRe-RDL) method [32] as described previously [33]. The gene sequence was confirmed after inserting the gene into the plasmid pET-24a (+) and transforming into BL21 (New England Biolabs, USA) cells. Expression and purification of ELPs was performed as previously described [33, 34]. ELPs were synthesized with a repeat sequence of [V-P-G-X-G]ₙ, which combined tyrosine and alanine as the guest amino acid ‘X’ in a 1:4 ratio as [(GAGVP)₂(GYGVP)(GAGVP)₂]ₙ. ELPs are abbreviated as Yn:Y₁₂ for n=12 and Y₂₄ for n=24.

Thermal characterization of the transition temperature (Tₜ). The optical density (OD) at 350 nm was analyzed for each ELP by varying the solution temperature using a UV-vis spectrophotometer (Cary 300, Varian Instruments, USA). ELP solutions at 25 μM concentration were heated at a rate of 1° C. per min from 4° C. to 60° C. and the OD of the sample was measured. The instrument was blanked with 1× PBS or deionized water (DW) each time before use. Tₜ was defined as the temperature corresponding to half the maximum OD [33].

Preparation of crosslinked Y₁₂ (CL_Y₁₂) and Y₂₄ (CL_Y₂₄) coated dishes. ELPs were crosslinked through tyrosine residues using tyrosinase (Tyr, 2 mg/ml; 150 unit/ml, mushroom tyrosinase, Sigma, USA). Briefly, 5, 7.5 or 10 μl of a stock of 10 mg/ml of two ELPs in DW was added to 500 μl of DW in a culture well, and the mixture was incubated with 1 μl of tyrosinase enzyme overnight at room temperature. The next day, supernatant was removed from ELPs, washed 2 times with DW, and cells were seeded on the surface. Tₜ of CL_Y₁₂ and CL_Y₂₄ was also obtained.

Maintenance of D3 mESCs and miPSCs. Mouse ES cell line D3 (American Type Culture Collection, CRL-1934, USA) was cultured without feeder cells on 0.1% gelatin-coated tissue culture plates (60 mm diameter, Falcon, Thermo Fisher Scientific, USA) in Dulbecco's minimum essential medium (DMEM, Thermo Fisher Scientific, USA) supplemented with 15% fetal bovine serum (FBS), 0.1% non-essential amino acids (NEAA, Sigma, USA), 100 U/mL leukemia inhibitory factor (LIF, ESGRO, Chemicon/Merck Millipore, Billerica, Mass., USA), and 50 μM β-mercaptoethanol (β-ME, Thermo Fisher Scientific, USA). The miPSCs (miPSCs #1) were used from stocks generated from mouse embryonic fibroblast cells as described previously [33]. Same culture media as the D3 mESCs was used to culture miPSCs.

Cell viability assay. Cell cytotoxicity assay was performed by using the cell counting kit-8 (CCK-8; Sigma, USA). ELPs were crosslinked at concentrations of 50, 75 or 100 μg/ml and D3 mESCs were seeded at a density of 10,000 cells per well. After 48 h of culture, the cell culture medium was aspirated, and the cells were washed with PBS one time followed by incubation with 10 μL of CCK-8 solution for 4 h at 37° C. The absorbance of solutions was measured at 450 nm on a SpectraMax M2 multimode microplate reader (Molecular Devices, Inc., USA).

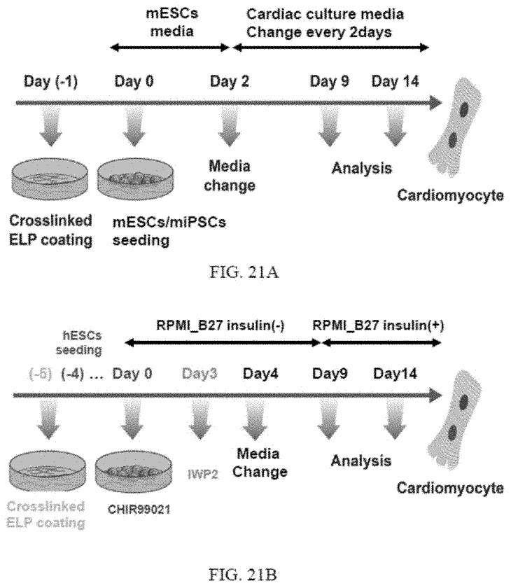

Direct cardiac differentiation of D3 mESCs and miPSCs. The scheme of direct cardiac differentiation of the mESCs and miPSCs is shown in FIG. 21A. To induce direct cardiac differentiation, mESCs or miPSCs were dissociated into single cells with TrypLE Express solution (Thermo Fisher Scientific, USA), and then 5×10⁴ cells were seeded into plates (Nunc 4-well culture dish, Thermo Fisher Scientific, USA) coated with crosslinked ELPs or gelatin or laminin. For laminin coating, Laminin-521 (LN521), a human recombinant isoform was obtained from Stem Cell Technology and coated on cell culture dishes by incubating 2 ml (1.25 μg/ml LN521 in PBS) overnight at 4° C. The mESCs or miPSCs were cultured in LIF-free ESC culture medium for 2 days and then replaced with Glasgow minimum essential medium (GMEM; Gibco USA) containing 2.5% knock serum replacement (KSR, Gibco, USA), 1% NEAA and 10 μM β-ME for next 12 days.

Quantitative real-time RT-PCR (qRT-PCR). qRT-PCR analysis was performed to analyze the expression of genes involved in cardiac differentiation. After 14 days of culture, total RNA was isolated from cultured cells using PureLink RNA Mini Kit (Invitrogen, Life Technologies, USA), and 1 μg total RNA from each group was reverse transcribed using the High-Capacity cDNA Reverse Transcription Kit (Applied Biosystems, USA) according to the manufacturer's instructions. PCR was performed on a Thermocycler (Applied Biosystems, USA) and quantitative real-time PCR on Quantstudio3 Real-time PCR system (Applied Biosystems, USA) using Power SYBR Green PCR Master Mix (Applied Biosystems, USA), and the mRNA levels were calculated using the comparative CT method with glyceraldehyde 3-phosphate dehydrogenase (GAPDH) as endogenous control. A list of primer sequences (5′-3′) used herein are shown in Table 1.

TABLE 1
List of qRT-PCR primers (SEQ ID NOS

| | | SEQ | | SEQ |
|---|---|---|---|---|
| Gene | forward primer | ID | reverse primer | ID |
| name | sequence (5′-3′) | NO | sequence (5′-3′) | NO |
| mcTNT2 | GAGGTGGTGGAGGAGT | 30 | CTACGTTGGCCTCCTC | 31 |
| | ACGA | | TGTC | |
| mNKX25 | AAGTGCTCTCCTGCTT | 32 | CACAGCTCTTTTTTAT | 33 |
| | TCCCAG | | CCGCCC | |
| mGATA4 | CCAGAAAACGGAAGCC | 34 | TGCTAGTGGCATTGCT | 35 |
| | CAA | | GGAGT | |
| mISL1 | TACCACATCGAGTGTT | 36 | TGGTCTGCACGGCAGA | 37 |
| | TCCGC | | AAA | |
| mMEF2C | CCAAATCTCCTCCCCC | 38 | TGATTCACTGATGGCA | 38 |
| | TATGA | | TCGTG | |
| mMESP1 | ACCCATCGTTCCTGTA | 40 | TCTAGAAGAGCCAGCA | 41 |
| | CGCAGA | | TGTCGC | |
| mMLC2v | ATTTGCTGCCCTAGGA | 42 | CCCAAACATCGTGAGG | 43 |
| | CGAGT | | AACAC | |
| mNPPA | GATCTGCCCTCTTGAA | 44 | AAGCTGTTGCAGCCTA | 45 |
| | AAGCA | | GTCCA | |
| mNPPB | CACCCAAAAAGAGTCC | 46 | TTGTGCCCAAAGCAGC | 47 |
| | TTCGG | | TTG | |
| mTBX5 | CTGGCCTTAATCCCAA | 48 | GCTTTGCCAGTTACGG | 49 |
| | AACGA | | ACCAT | |
| mTBX18 | ATGATCATCACCAAAG | 50 | CGGCACAATATCCATG | 51 |
| | CCGG | | GCA | |
| mWT1 | CTCCCAGCTTGAATGC | 52 | TGCCCTTCTGTCCATT | 53 |
| | ATGAC | | TCACT | |
| mTUJ1 | TCAGCGATGAGCACGG | 54 | GTCGTGGTTCTCTCCT | 55 |
| | CATA | | TCTA | |
| mAFP | TGATCCAACTAGGCTT | 56 | CCAGATTGTCCTACAT | 57 |
| | CTGC | | CCA | |
| mOCT4 | TCAGACTTCGCCTTCT | 58 | TTCCACTCGTGCTCCT | 59 |
| | CACC | | GCCT | |
| mNANOG | CAGATGCAACTCTCCT | 60 | AATTCACCTCCAAATC | 61 |
| | C | | ACTG | |
| mGAPDH | TCCAGTATGACTCCAC | 62 | GGCTAAGCAGTTGGTG | 63 |
| | TCAC | | GT | |
| hcTNT2 | AAGAGGCAGACTGAGC | 64 | AGATGCTCTGCCACAG | 65 |
| | GGGAAA | | CTCCTT | |
| hGAPDH | GTCTCCTCTGACTTCA | 66 | ACCACCCTGTTGCTGT | 67 |
| | ACAGCG | | AGCCAA | |

Immunocytochemistry. Direct differentiation procedure was performed by culturing and differentiating the cells on glass coverslips. At day 14, cells were fixed in 4% paraformaldehyde solution, permeabilized by 0.1% Triton X-100 for 15 min at RT, blocked using 10% normal goat serum (Jackson Immuno Research, USA) at RT, and stained overnight at 4° C. with mouse monoclonal α-smooth muscle actin (SMA, Abcam, U1A) or rabbit polyclonal cardiac Troponin T2 (cTNT2, Invitrogen, USA) or mouse monoclonal α-Actinin (Actinin, Sigma, USA) antibodies. Secondary antibody reaction was performed with anti-mouse-IgG-AlexaFluor-488 or 594 (1:500, Invitrogen, USA) or goat anti-rabbit tetramethylrhodamine isothiocyanate (TRITC, 1:500, Invitrogen, USA) at RT for 60 min. Nuclei were stained with, 4′,6-diamidino-2-phenylindole (DAPI, Vector lab, USA). Samples were examined using Nikon C2 confocal microscope.

Flow cytometry. After 14 days of culture, cells were dissociated with TrypLE Express solution and single cells were fixed with 4% paraformaldehyde. Cell were permeabilized in 0.05% Triton X-100 in 3% bovine serum albumin (BSA) for 1 hr at room temperature and stained with cTNT2 antibody. For negative controls, cTNT2 antibody was omitted. After staining with primary antibody, cells were washed in PBS containing 3% BSA and goat anti-rabbit TRITC (1:500) was added and incubated for 1 hr at 4° C. After washing three times with washing solution, cells were analyzed using flow cytometry. Cell debris was gated out and 10,000 events were acquired for analysis, which was performed using FlowJo software (FlowJo LLC, Ashland, USA).

Visualization of Ca²⁺ flux during cardiomyocyte contraction. Intracellular calcium (Ca²⁺) oscillations during cardiomyocyte contraction were imaged using a protocol described earlier [35]. Briefly, after 14 days of culture, the cells were incubated with 1 μM Fluo-4 AM (Invitrogen, USA) for 40 min at 37° C., followed by three washings with PBS solution. After changing to the pre-warmed culture medium, intracellular Ca²⁺ transients were video recorded using Nikon C2 confocal microscope and a Clara CCD Camera (1392×1040, air cooled 30 C to −45 C). Nikon NIS-Elements, Advanced Research Acquisition and Analysis Package (ver 4.13.10) was then used to mark region of interest (ROI) in a single cell on the video. A module in the package was used to extract ‘Intensity vs time’ data from the ROI. The data was exported and processed using Microsoft Excel to compute time interval between fluorescent intensity peaks, which was reported as ‘calcium peak time’.

Microarray. Total RNA was isolated from mESCs at day 9 and 14 of differentiation with a PureLink RNA mini kit (Invitrogen). Gene expression analysis was conducted using Agilent Whole Mouse Genome 4×44 multiplex format oligo arrays (014868) (Agilent Technologies) following the Agilent 1-color microarray-based gene expression analysis protocol. Starting with 500 ng of total RNA, Cy3-labeled cRNA was produced following the manufacturer's protocol. For each sample, 1.65 of Cy3-labeled cRNAs were fragmented and hybridized for 17 hrs in a rotating hybridization oven. Slides were washed and then scanned with an Agilent Scanner. Data was obtained using the Agilent Feature Extraction software (v12), using the 1-color defaults for all parameters. The Agilent Feature Extraction Software performed error modeling, adjusting for additive and multiplicative noise. To identify differentially expressed probes, an analysis of variance (ANOVA) was used to determine if there was a statistical difference between the means of groups. In addition, the inventors used a multiple test correction to reduce the number of false positives. Specifically, an ANOVA and Bonferroni multiple test correction with a p value of p<0.05 was performed using OmicSoft Array Studio (Version 10) software. The microarray data discussed in this study have been deposited in the National Center for Biotechnology Information's Gene Expression Omnibus (www.ncbi.nlm.nih.gov/geo/) and are accessible through GEO Series accession number GSE145623.

Functional and pathway enrichment analysis. Pathway analyses were performed using ConsensusPathDB (cpdb.molgen.mpg.de/MCPDB) and REACTOME pathway annotations. Analysis of the GO biological process was performed with David Bioinformatics Resources [36]. A threshold of FDR <1% or p value <0.01 was used to select for statistically significant categories.

Differentiation of H9 hESCs into cardiomyocytes. H9 hESCs were obtained from WiCell Research Institute (Madison, Wis., WiCell) and adapted to single cell, non-colony type monolayer culture as previously described [37]. hESCs were dissociated with 1× Accutase (Innovative Cell Technologies, USA) for 5 min at 37° C. and subsequently resuspended in mTeSR1 defined medium (Stem Cell Technologies, Canada), and centrifuged at 210×g for 3 minutes. For maintenance of hESCs, dissociated single cells were then plated at a density of 2×10⁵ cells/well in 6-well dishes coated with hESCs-Qualified Matrigel (Corning, USA) in mTeSR1 containing 10 μM ROCK inhibitor (#1254, Tocris Bioscience, USA). For cardiac differentiation [38], hESCs were transferred to 6 well plates coated either with Matrigel (Growth Factor Reduced Membrane Matrix, 354230, Corning, USA) or CL_Y₁₂. For coating with Matrigel the Growth Factor Reduced Membrane Matrix Matrigel was diluted to 1:100 in ice cold DMEM/F-12 (Thermo Fisher Scientific, USA) and added to wells of the 6 well plates. Coated dishes were incubated overnight at 4° C. For coating with CL_Y₁₂, 75 μg/ml was used as described earlier in the materials and methods section. The hESCs were seeded in Matrigel and CL_Y₁₂ coated wells at a density of 2.4×10⁴ cells/cm² using mTeSR1 supplemented with 10 μM Rock inhibitor for 24 h. After 24 hrs, the medium was changed with freshly prepared mTeSR1 without Rock inhibitor and medium was exchanged every day. After three or four days, when the confluency of the culture reached 80-90%, cells were treated with 6-8 μM of GSK3β inhibitor CHIR99021 (#4423, Tocris Bioscience, USA) in RPMI 1640 (#61,870, Thermo Fisher Scientific, USA) supplemented with B27-without insulin (#A1895601, Thermo Fisher Scientific, USA). After 24 h the medium was changed using RPMI-B27 (without insulin) to remove CHIR99021 and cultured continuously for 48 h. On day 3 cells were treated with 5 μM Wnt inhibitor IWP2 (#3533, Tocris Bioscience, USA) diluted in RPMI-B27 without insulin and incubated for 48 h. On day 5 medium was changed to freshly prepared RPMI-B27 without insulin and cells were cultured for 48 h. At day 7-9 medium was changed to RPMI-B27 with insulin (#17,504,044, Thermo Fisher Scientific, USA), and medium was changed every two days thereafter till day 15. The scheme of direct cardiac differentiation of the hESCs is shown in FIG. 21B.

Statistical analysis. Data were expressed as mean and standard error of mean (SEM). Statistical significance was determined by unpaired two-tailed Student's t-test. All results were derived from three or more independent experiments.

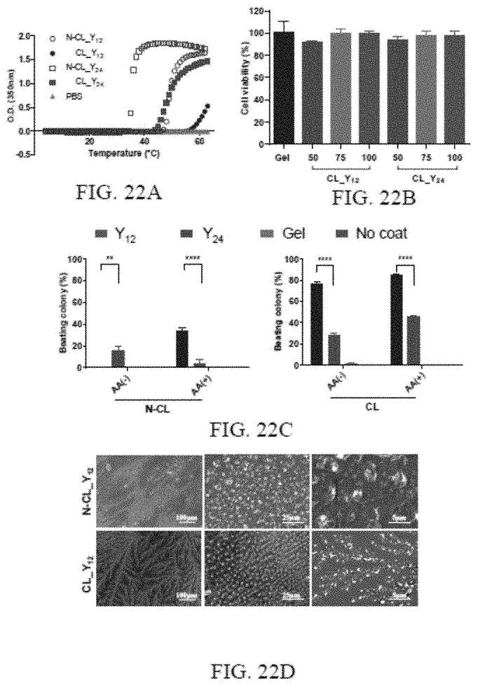

Design and characterization of ELPs. To perform direct differentiation of stem cells into cardiomyocytes, the inventors first coated ELPs on the culture dish surface. There are two ways of coating ELPs onto surfaces, either by physical adsorption or by chemical crosslinking. Physical adsorption can be facilitated if the ELPs have a Tₜ close to room temperature (or the cell culture temperature). This means they can spontaneously aggregate and adhere to the culture dish surface at room temperature. However, if the Tₜ of ELPs is significantly higher than room temperature, then the ELPs require a higher temperature to aggregate and will resolubilize as soon as the temperature is lowered; therefore, they must be chemically crosslinkable so that they can be crosslinked to lay down insoluble aggregates on surfaces. The inventors attempted to reduce the Tₜ of ELPs by selecting hydrophobic amino acids as the guest residue ‘X’ in [V-P-G-X-G]ₙ because it is known that hydrophobic residues decrease the Tₜ. The inventors combined tyrosine and alanine as the guest amino acids in a 1:4 ratio creating ELPs with the repeating unit of [(GAGVP)₂(GYGVP)(GAGVP)₂]ₙ (n=12 or 24). In this design, tyrosine served a dual role; it not only served as a hydrophobic residue but also as the crosslinking residue through action of an enzyme called tyrosinase. MALDI analysis confirmed the molecular weights of the two ELPs. The Tₜ of non-crosslinked Y₁₂ ELPs (N-CL_Y₁₂) was observed to be 49.3° C., while that of non-crosslinked Y₂₄ (N-CL_Y₂₄) was 35.6° C. (FIG. 22A). Upon crosslinking the Tₜ of Y₁₂ (CL_Y₁₂) and Y₂₄ ELPs (CL_Y₂₄) increased to 56.2° C. and 49.1° C., respectively (FIG. 22A).

Cell viability on crosslinked ELPs. Next, cell viability was examined on ELP-coated surfaces. Because the Tₜ of Y₁₂ ELPs is 49.3° C., which is much higher than the cell culture temperature, the Y₁₂ ELPs will remain in solution and cannot form coatings by physical adsorption. Therefore, Y₁₂ and Y₂₄ ELPs were crosslinked using tyrosinase and investigated viability of D3 mESCs by culturing them for 2 days over the crosslinked ELPs. ELPs were crosslinked at concentrations of 50, 75 or 100 μg/ml. As shown in FIG. 22B, similar to the gelatin control, both CL_Y₁₂ and CL_Y₂₄ showed high cell viability at all concentrations of crosslinked ELPs.

Comparison of N-CL and CL ELP coatings for cardiac differentiation of mESCs. After determining that CL_Y₁₂ and CL_Y₂₄ were not cytotoxic to D3 mESCs, the inventors next evaluated whether they can promote differentiation of D3 mESCs. For this analysis the inventors opted to coat the ELPs at 75 μg/ml concentration. To dissect the importance of crosslinking, the inventors produced coatings of Y₁₂ and Y₂₄ under both N-CL and CL conditions. Since ascorbic acid (AA) is commonly added to cardiac differentiation media to enhance cardiac differentiation and promote the proliferation of cardiac progenitor cells [39], the inventors also examined the effect of AA on cardiac differentiation. When ELPs were not crosslinked, no beating colonies were observed for N-CL_Y₁₂ and 16% beating colonies were observed in the N-CL_Y₂₄ group (FIG. 22C). The addition of AA significantly increased the colonies for N-CL_Y₁₂ group to 30% but had no significant increase for the N-CL_Y₂₄ group. In the case of crosslinked ELP coatings, the CL_Y₁₂ coated dish showed 76% and 85% beating cardiomyocyte colonies in AA(−) and AA(+) groups, respectively (FIG. 22C). In comparison, the CL_Y₂₄ coating showed 28% and 45% beating cardiomyocyte colonies for AA(−) and AA(+) groups, respectively. Only a few (<2%) beating colonies were observed in gelatin control and non-coated dishes. This data demonstrates that crosslinking the ELPs produced a significantly higher number of beating colonies. To study the cause of this phenomenon, the inventors used SEM to examine crosslinked and non-crosslinked coatings produced on culture dish surfaces. FIG. 22D shows that CL_Y₁₂ produce coatings with a branched pattern that more uniformly cover the surface as compared to N-CL_Y₁₂ and Y₂₄ (CL and N-CL). This difference in surface coatings could be due to the high Tₜ of N-CL_Y₁₂ over N-CL_Y₂₄, which can affect their aggregation pattern at the culture temperature (37° C.).

Characterization of cardiac differentiation on CL_Y₁₂ coatings. Having identified that CL_Y₁₂ is better than CL_Y₂₄ with respect to cardiomyocyte differentiation, the inventors next sought to perform a more thorough characterization of cardiomyocytes differentiated on Y₁₂ ELPs. The inventors first differentiated D3 mESCs on CL_Y₁₂ that was crosslinked at 50, 75 or 100 μg/ml concentrations. After 2 days of culture, the ES morphology showed a slight monolayer formation at 50 μg/ml, whereas at 75 μg/ml and 100 μg/ml, many colony-like clusters were observed (FIG. 23A). On day 2, the number of cell colony clusters increased with increase in concentration of CL_Y₁₂ (FIG. 23A), however, for the laminin control group (LN521), only monolayer structures and no clusters were observed (FIG. 23A). On the 9th day after seeding, the beating rate of colonies was counted. The beating frequencies were between 70 and 90 beats/min for each concentration of CL_Y₁₂ and LN521(FIG. 23B). Although, the beating rate of the derived cardiomyocytes on the LN521 coated dish was similar to the cardiomyocytes on CL_Y₁₂ coated dish, the total cardiomyocytes on the LN521 coated dish were very sparse. Furthermore, the beating rate and mechanical contraction for LN521 cardiomyocytes significantly reduced on day 14. In contrast, a strong contraction of cardiomyocytes was observed on day 14 for cardiomyocytes differentiated from CL_Y₁₂ (75 μg/ml), while the contractions in 50 μg/ml and 100 μg/ml CL_Y₁₂ groups were qualitatively weaker.

To visualize calcium flux in cardiomyocytes during mechanical contractions, the inventors used a fluorescent intracellular calcium sensor, Fluo-4 AM. The time between calcium intensity peaks was similar for cardiomyocytes obtained from 50 and 75 μg/ml CL_Y₁₂ but it was significantly lower for cardiomyocytes obtained from 100 μg/ml CL_Y₁₂ (FIG. 23C). In other words, 50 or 75 μg/ml CL_Y₁₂ produced cardiomyocytes that beat faster as compared to 100 μg/ml CL_Y₁₂. Although the difference between 50 and 75 μg/ml was not statistically significant, the beating of cardiomyocytes was marginally faster in the 75 μg/ml group. The inventors selected 75 μg/ml CL_Y₁₂ for further evaluation.

To obtain a better insight into the differentiation quality the inventors performed qRT-PCR to measure cardiomyocyte (cTNT2) and other lineage markers (Tuj1: ectoderm, AFP: endoderm). FIG. 23D shows that for LN521 coated dish the relative expression of cTNT2 (cardiomyocyte marker) is significantly low as compared to 75 μg/ml CL_Y₁₂ coated dish. Furthermore, non-cardiomyocyte lineage RNA expression was significantly higher in LN521 coated dish as compared to CL_Y₁₂ coated dish.

Immunostaining and flow cytometry analysis of cardiomyocytes grown on CL_Y₁₂. To visualize cardiomyocyte-specific marker expression, D3 mESCs were induced into cardiomyocyte lineage on a CL_Y₁₂ (75 μg/ml) coated dish without AA. Double immunofluorescent staining for cTNT2 and SMA was performed and FIG. 23E shows a higher positive staining of cTNT2 in the CL_Y₁₂ coated dish as compared to LN521 coated dish. Immunostaining for SMA did not produce significant signal intensity. Flow cytometric analysis of day 14 cardiomyocytes showed about 93% of the cardiomyocytes contained cTNT2, a cardiac specific marker (FIG. 23F). These results suggest that CL_Y₁₂ significantly improves cardiac differentiation of mESCs as compared to LN521. (FIG. 23G) FACS analysis of cTNT2 expression in cardiomyocytes differentiated from D3 mESCs on CL_Y₁₂ (75 μg/ml) coated dishes without AA. gray color: isotype control. *: p<0.05 .

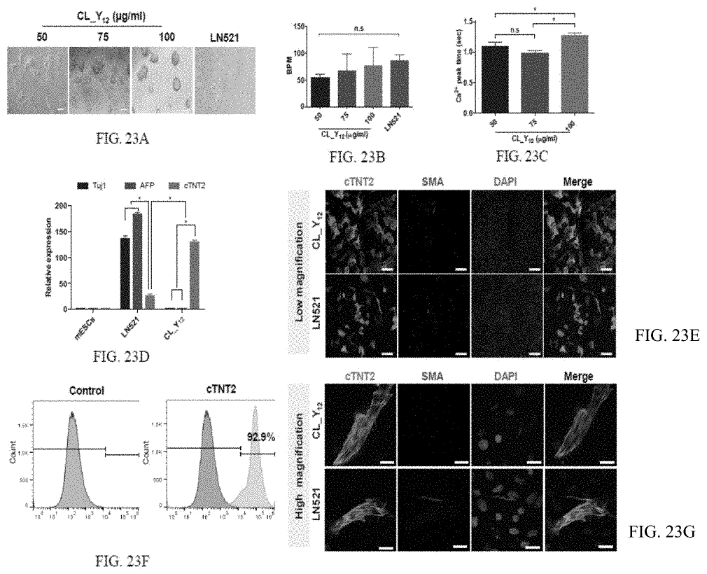

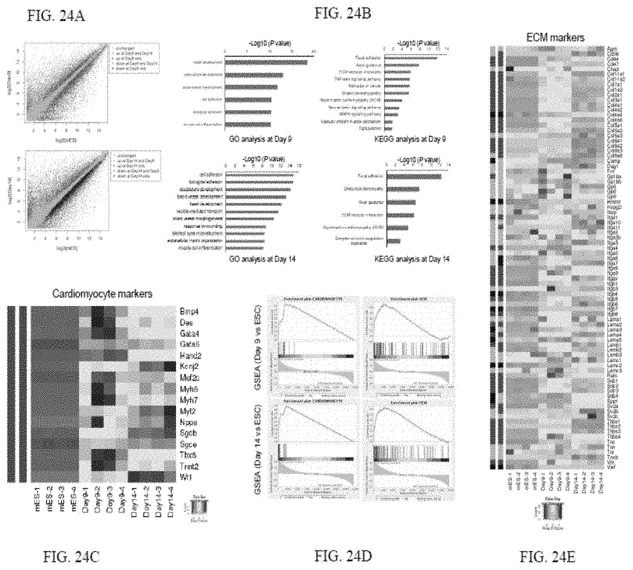

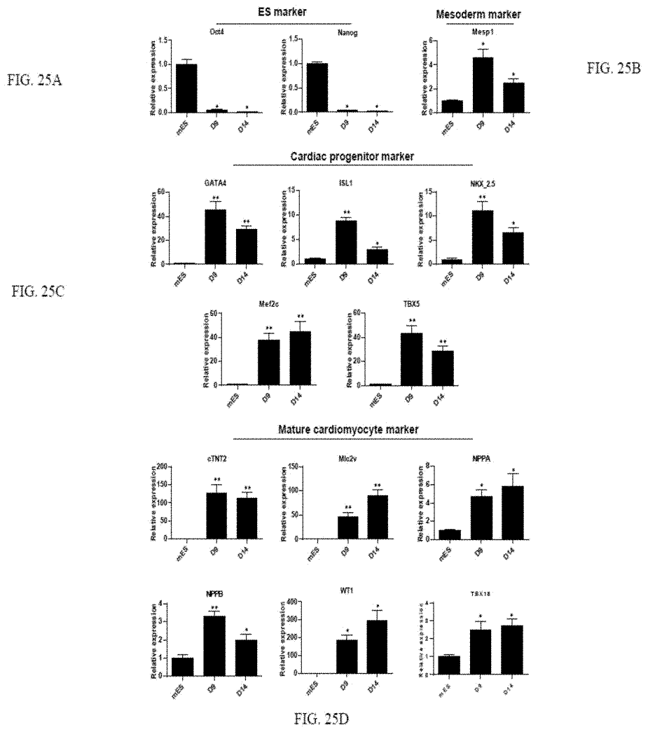

CL_Y₁₂ specifies cardiomyocyte phenotype through the activation of ECM genes. Microarray analysis was performed to examine changes in gene expression at day 9 and 14 of the CL_Y₁₂-induced differentiation of mESCs into cardiomyocytes compared to untreated control mESCs. Analysis of global gene expression showed that at day 9 and 14 about 3000 genes were significantly up- or down-regulated (FIG. 24A). Analysis of genes significantly up-regulated (FDR 0.05) during CL_Y₁₂-induced cardiac differentiation revealed that genes related to heart development, focal/cell adhesion, and ECM are among the top pathways (FIG. 24B). As shown in the heatmap in FIG. 24C, a number of cardiomyocyte-associated genes, including Bmp4, Gata4, Gata6, Myh6, Myl2, Nppa, Sgcb, Wt1, and Ctnt2, are significantly induced during CL_Y₁₂ -induced differentiation. To further confirm the enrichment of cardiomyocyte and ECM genes in differentiated cells compared to mESC, the inventors performed gene set enrichment analysis (GSEA) based on pathway analysis. Consistently, genes in both Gene Ontology (GO) and Kyoto Encyclopedia of Genes and Genomes (KEGG) were positively enriched in cardiomyocyte differentiated cells at both day 9 and 14 (FIG. 24D). ECM has been reported to play a critical role in cardiac differentiation of ESCs [40]. This examination of the gene expression profiles of the ECM pathway-associated components confirmed the induction of ECM genes, including several collagen and integrin genes, Lama, Lamb, Sdc, Thbs, and Vtn, during cardiomyocyte induction (FIG. 24E). The changes in gene expression during CL_Y₁₂-induced differentiation was supported by qRT-PCR analysis. The expression of undifferentiated ESC markers (Oct4 and Nanog) was repressed during CL_Y₁₂-induced differentiation, whereas the expression of mesoderm markers was greatly enhanced (FIGS. 25A and 25B). Moreover, consistent with the induction of cardiac differentiation, the expression of several cardiomyocyte progenitor and maturation markers was significantly induced at both day 9 and 14 (FIGS. 25C and 25D). These observations support this conclusion that CL_Y₁₂ coating promotes mESC differentiation into cardiomyocytes likely through ECM signaling pathway. With respect to the maturation of cardiomyocytes, the inventors also checked gene expression of maturation markers of cardiomyocyte. In rodents, during maturation, β-MHC (MYH7) expression switches to α-MHC (MYH6), and as a result the MYH6 to MYH7 ratio increases [41]. Indeed, from the microarray data the inventors saw a 1.6 fold increase in MYH6 to MYH7 ratio from day 9 to day 14. Similarly, in mature rodent cardiomyocytes the TNNI1 switches to TNNI3, and the TNNI3 to TNNI1 ratio was found to increase 5.4-fold from day 9 to day 14.

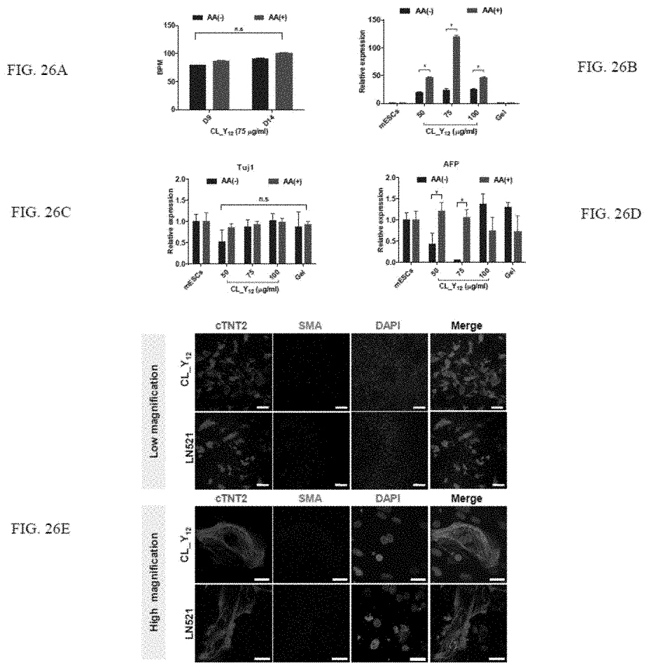

Effect of AA on direct differentiation of mESCs into cardiomyocytes. Although CL_Y₁₂ coating promoted cardiac differentiation without use of any cardiac inducers such as AA, BMP, Activin A and TGF, the inventors were curious to know if any synergy could be achieved if cardiac inducers were used during the differentiation process. Therefore, the inventors selected AA and differentiated D3 mESCs on 75 μg/ml CL_Y₁₂ coated dishes with and without 100 μM AA from day 2 of differentiation to day 14. Although the beating rate gradually increased from day 9 until day 14 for both AA(−) and AA(+) groups, this effect was not statistically significant (p>0.05, FIG. 26A). Upon addition of AA, gene expression of one of the major mature cardiomyocyte markers, cTNT2 was strongly increased (FIG. 26B). Coinciding with increase in cTNT2, the specific gene expression marker of ectoderm (Tuj1) and endoderm (AFP) lineages were very low in each group and similar to undifferentiated D3 mESCs (FIGS. 26C, 26D). Strong expression of cTNT2 in cardiomyocytes at day 15 was confirmed by immunocytochemistry (FIG. 26E). Expression of SMA expression could not be identified in immunohistochemistry.

Cardiac differentiation of miPSCs. Since iPSCs can provide a patient-specific cell source and are less immunogenic than ESCs, the inventors wanted to check whether this crosslinked ELPs-based cardiac differentiation method can also be applied to iPSCs. Therefore, the inventors used a miPSCs line (#1), which the inventors had previously produced by an ELP-based gene delivery system and induced differentiation following the same culture protocol as for mESCs. Cardiomyocytes that were generated on CL_Y₁₂ from miPSCs #1 cell lines showed similar beating colony numbers (FIG. 27A) and beating rates (FIG. 27B) as the cardiomyocytes obtained from D3 mESCs. Addition of AA did not significantly enhance the cardiomyocyte beating rate (FIG. 27B). In addition, cardiomyocytes derived from miPSCs #1 showed early (Mesp1) and late (cTNT2 and TBX₁₈) gene marker expression similar to cardiomyocytes derived from D3 mESCs (FIG. 27C). In accordance with high gene expression, immunocytochemistry was able to visually confirm strong protein expression of cTNT2 in cardiomyocytes generated from D3 and miPSCs #1 (FIG. 27D). Flow cytometric analysis of cardiomyocytes derived from IPS#1 was performed using cTNT2 as a cardiac specific marker on day 14 post-differentiation to determine the differentiation rate of cardiomyocytes on the crosslinked Y₁₂ ELPs coated dish (FIG. 27E). About 65% cardiomyocytes were generated from miPSCs #1 without AA, and this number increased to 77% when AA was added to the media. This data shows that CL_Y₁₂ enabled cardiac differentiation from both mESCs and miPSCs.

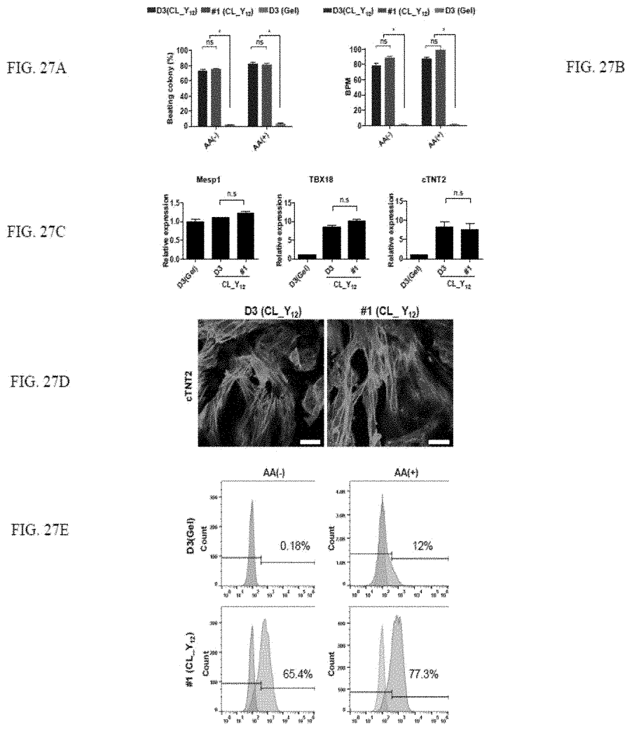

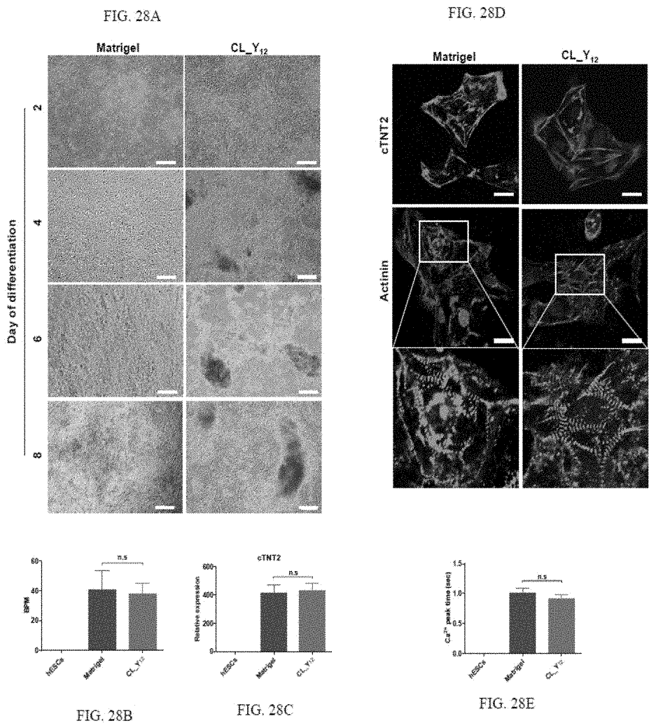

Cardiac differentiation of hESCs. To check whether the crosslinked ELPs-based cardiac differentiation method can also be applied to hESCs the inventors differentiated H9 hESCs on CL_Y₁₂ coated dish. On day 8-12 of differentiation, cardiac differentiation was confirmed. Cardiomyocyte-like morphology and spontaneously contracting cell clusters, which are typical characteristics of cardiomyocytes, were seen in Matrigel and CL_Y₁₂ coated dishes (FIG. 28A, but not in LN521 coated dish. Similar beating rates were observed for cardiomyocytes generated on Matrigel and CL_Y₁₂ (FIG. 28B). In addition, cardiomyocytes derived from Matrigel and CL_Y₁₂ showed similar expression of cardiac marker (cTNT2) (FIG. 28C). In accordance with high gene expression, immunocytochemistry was able to visually confirm strong protein expression of cTNT2 and Actinin in cardiomyocytes generated from Matrigel and CL_Y₁₂ (FIG. 28D). Ca²⁺ influx showed similar cardiomyocyte contractility in cardiomyocytes derived from both Matrigel and CL_Y₁₂ (FIG. 28E).

In this example, ELPs, a recombinant elastin mimicking molecule was used for cardiac differentiation of stem cells. The inventors designed an ELP containing tyrosine as one of the hydrophobic guest residue to exploit the fact that the enzyme called tyrosinase can be used to crosslink the ELPs, thus allowing the inventors to investigate coatings produced by physical adsorption and chemical crosslinking [42]. The inventors prepared two ELPs namely, Y₁₂ and Y₂₄, with Y₂₄ being twice as large as Y₁₂. Through culture of mESCs, miPSCs and hESCs on crosslinked ELPs, and developed a protocol by which these cells can be differentiated into cardiomyocytes within 2 weeks.

To date, many research groups [43-49] have published a number of protocols to differentiate ES cells into cardiomyocytes. Although several protocols based on EB-formation [7, 50-53] have shown high cardiomyocyte yields, it is often observed that yields vary between batches. In addition, this technique has a significant limitation because the growth factors required for differentiation of stem cells do not equally affect cells deep within the EB, resulting in a significant change in efficiency [45]. For this reason, the inventors opted to use the EB-independent monolayer differentiation protocol, and without cardiomyocyte differentiation factors such as bone morphogenetic protein, noggin, activin, and AA. Some groups have shown spontaneously beating cardiomyocytes derived from adipose-derived murine stromal vascular cells using gelatin hydrogels. These cells showed similar character as naïve cardiomyocytes with regards to gene expression of cardiomyocyte markers, beating mode, calcium activity and cTNT2 protein expression, but the yield was very low (14%) [54-56].

In this system, CL_Y₁₂ was used and found that mESCs, miPSCs and hESCs can be differentiated into cardiomyocyte cells. The cardiac differentiation effect was found to be dependent on crosslinking of ELPs because without crosslinking both the Y₁₂ and Y₂₄ ELPs generated low cardiomyocyte yields. By way of explanation, and in no way a limitation of the present invention, it is possible that physically adsorbed coatings are unstable and detach from the culture surface over time, which might lead to poor yields. This detachment phenomenon will be more prominent for Y₂₄ ELP since it's Tₜ of 35.6° C. is close to the cell culture temperature. The inventors observed that Y₂₄ ELP was not as effective as Y₁₂ ELPs in inducing cardiomyocyte differentiation and its yield was almost half as compared to Y₁₂ ELPs. The reason for this effect is not evident, but it could be due to the differences in micro-and-nanostructure of coatings produced by Y₂₄ as compared to Y₁₂ ELP. The inventors observed that the Y₁₂ ELP produced a branched pattern that more uniformly covered the surface as compared to Y₂₄ ELP.

In order to evaluate cardiomyocyte function, the beating rate, Ca²⁺ influx was examined, and cardiomyocyte marker expression at different Y₁₂ ELPs concentrations (50, 75 and 100 μg/ml). The beating rate of cardiomyocytes on ELPs-crosslinked dishes increased on day 9-10 of culture, which was accompanied by a strong and significant increase in cTNT2 expression. CL_Y₁₂ at 75 μg/ml concentration provided the least time-to-peak indicating that cardiomyocytes at this condition were beating faster than cardiomyocytes at 50 and 100 μg/ml.

The gene profiling analyses shows that that CL_Y₁₂ efficiently induces differentiation of mESCs into cardiomyocytes. This is further supported by data showing that CL_Y₁₂ markedly enhances the expression of a number of ECM genes and cardiomyocyte lineage markers. For example, the TNNI3 to TNNI1 ratio and the MYH6 to MYH7 ratio increased significantly from day 9 to day 14 culture, indicating gain of higher cardiomyocyte maturity. Several studies have demonstrated the importance of the ECM and integrins in the regulation of cardiac differentiation, function, and contractibility [57-59], and cationic polymer-coated surfaces have been reported to enhance myocardial cell contraction [50]. Thus, the induction of cardiac differentiation by CL_Y₁₂ might at least in part be mediated through activation of ECM and integrin signaling pathways.

The ELP-based monolayer differentiation method showed about 65-77% yield from miPSCs, which is similar to other protocols [4, 60, 61]. Since AA increased cardiomyocyte yield, it is possible to speculate that the yield from ELP-based approach can be further increased by adding growth factor cocktails used in other protocols [4]. It is also possible to combine different ELP designs, which can work synergistically to increase cardiomyocyte yield without addition of expensive growth factors. For example, ELPs can be designed with different ‘X’ guest residues, or signaling ligands such as RGD could be attached to ELPs to enhance cardiac differentiation. ELP-RGD fusion molecules have already been used in the field of tissue engineering [62].

It is also shown herein that CL_Y₁₂ showed similar effects in inducing hESC differentiation to cardiomyocytes. According to Burridge et. al, Laminin-521 and Laminin-511 showed a similar cardiac differentiation rate as compare to Matrigel (cardiomyocyte purity >80%) in hESCs and hiPSCs [38]. CL_Y₁₂ showed that the gene expression (cTNT2) and beating rates are similar to Matrigel, but the inventors could not obtain similar results using Laminin-521.

Thus, the present invention can be used to induce cardiomyocytes from stem cells using crosslinked ELPs. The crosslinked ELP system demonstrated that spontaneously pulsating cardiomyocyte-like cells could be derived from mESCs, miPSCs, hESCs. Immunofluorescent staining of proteins and mRNA expression levels of cardiac markers, and cytoplasmic calcium transient activity confirmed the development of cardiomyocytes. However, the precise mechanism of cell differentiation in the presence of crosslinked ELPs is not yet known, and molecular and functional characteristics in differentiated myocardial cells may require further investigation in the future. ELPs are an attractive alternative to Matrigel since ELPs provide a well-defined and well-characterized material that can be produced under a reproducible and controllable environment. Crosslinked ELPs-based differentiation method can be used with myocardial cell therapy and can also be used for the differentiation of stem cells into other lineages.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES—EXAMPLE 1

1. Serra, M., C. Brito, C. Correia, and P. M. Alves, Process engineering of human pluripotent stem cells for clinical application. Trends in Biotechnology, 2012. 30(6): p. 350-359.

2. McNeish, J., Embryonic stem cells in drug discovery. Nat Rev Drug Discov, 2004. 3(1): p. 70-80.

3. Yin, X., Benjamin E. Mead, H. Safaee, R. Langer, Jeffrey M. Karp, and O. Levy, Engineering Stem Cell Organoids. Cell Stem Cell. 18(1): p. 25-38.

4. Jing, D., A. Parikh, J. M. Canty, Jr., and E. S. Tzanakakis, Stem cells for heart cell therapies. Tissue Eng Part B Rev, 2008. 14(4): p. 393-406.

5. Tzanakakis, E. S., D. J. Hess, T. D. Sielaff, and W. S. Hu, Extracorporeal tissue engineered liver-assist devices. Annu Rev Biomed Eng, 2000. 2: p. 607-32.

6. Lindvall, O., Z. Kokaia, and A. Martinez-Serrano, Stem cell therapy for human neurodegenerative disorders-how to make it work. Nat Med, 2004. 10 Suppl: p. S42-50.

7. Zujur, D., K. Kanke, A. C. Lichtler, H. Hojo, U. I. Chung, and S. Ohba, Three-dimensional system enabling the maintenance and directed differentiation of pluripotent stem cells under defined conditions. Sci Adv, 2017. 3(5): p. e1602875.

8. Feng, J., K. Mineda, S. H. Wu, T. Mashiko, K. Doi, S. Kuno, K. Kinoshita, K. Kanayama, R. Asahi, A. Sunaga, and K. Yoshimura, An injectable non-cross-linked hyaluronic-acid gel containing therapeutic spheroids of human adipose-derived stem cells. Sci Rep, 2017. 7(1): p. 1548.

9. Lei, Y., D. Jeong, J. Xiao, and D. V. Schaffer, Developing Defined and Scalable 3D Culture Systems for Culturing Human Pluripotent Stem Cells at High Densities. Cell Mol Bioeng, 2014. 7(2): p. 172-183.

10. Chayosumrit, M., B. Tuch, and K. Sidhu, Alginate microcapsule for propagation and directed differentiation of hESCs to definitive endoderm. Biomaterials, 2010. 31(3): p. 505-14.

11. Serra, M., C. Correia, R. Malpique, C. Brito, J. Jensen, P. Bjorquist, M. J. T. Carrondo, and P. M. Alves, Microencapsulation Technology: A Powerful Tool for Integrating Expansion and Cryopreservation of Human Embryonic Stem Cells. PLOS ONE, 2011. 6(8): p. e23212.

12. Gerlach, J. C., M. Hout, J. Edsbagge, P. Bjorquist, M. Lubberstedt, T. Miki, H. Stachelscheid, E. Schmelzer, G. Schatten, and K. Zeilinger, Dynamic 3D culture promotes spontaneous embryonic stem cell differentiation in vitro. Tissue Eng Part C Methods, 2010. 16(1): p. 115-21.

13. Come, J., X. Nissan, L. Aubry, J. Tournois, M. Girard, A. L. Perrier, M. Peschanski, and M. Cailleret, Improvement of culture conditions of human embryoid bodies using a controlled perfused and dialyzed bioreactor system. Tissue Eng Part C Methods, 2008. 14(4): p. 289-98.

14. Ouyang, A. and S. T. Yang, A two-stage perfusion fibrous bed bioreactor system for mass production of embryonic stem cells. Expert Opin Biol Ther, 2008. 8(7): p. 895-909.

15. Nie, Y., V. Bergendahl, D. J. Hei, J. M. Jones, and S. P. Palecek, Scalable culture and cryopreservation of human embryonic stem cells on microcarriers. Biotechnol Prog, 2009. 25(1): p. 20-31.

16. Abranches, E., E. Bekman, D. Henrique, and J. M. Cabral, Expansion of mouse embryonic stem cells on microcarriers. Biotechnol Bioeng, 2007. 96(6): p. 1211-21.

17. Steiner, D., H. Khaner, M. Cohen, S. Even-Ram, Y. Gil, P. Itsykson, T. Turetsky, M. Idelson, E. Aizenman, R. Ram, Y. Berman-Zaken, and B. Reubinoff, Derivation, propagation and controlled differentiation of human embryonic stem cells in suspension. Nat Biotech, 2010. 28(4): p. 361-364.

18. Fok, E. Y. and P. W. Zandstra, Shear-controlled single-step mouse embryonic stem cell expansion and embryoid body-based differentiation. Stem Cells, 2005. 23(9): p. 1333-42.

19. Sen, A., M. S. Kallos, and L. A. Behie, Effects of Hydrodynamics on Cultures of Mammalian Neural Stem Cell Aggregates in Suspension Bioreactors. Industrial & Engineering Chemistry Research, 2001. 40(23): p. 5350-5357.

20. Cherry, R. S. and K. Y. Kwon, Transient shear stresses on a suspension cell in turbulence. Biotechnol Bioeng, 1990. 36(6): p. 563-71.

21. Lei, Y. and D. V. Schaffer, A fully defined and scalable 3D culture system for human pluripotent stem cell expansion and differentiation. Proceedings of the National Academy of Sciences of the United States of America, 2013. 110(52): p. E5039-E5048.

22. Rozario, T. and D. W. DeSimone, The extracellular matrix in development and morphogenesis: a dynamic view. Dev Biol, 2010. 341(1): p. 126-40.

23. Miner, J. H., C. Li, J. L. Mudd, G. Go, and A. E. Sutherland, Compositional and structural requirements for laminin and basement membranes during mouse embryo implantation and gastrulation. Development, 2004. 131(10): p. 2247-56.

24. Noakes, P. G., M. Gautam, J. Mudd, J. R. Sanes, and J. P. Merlie, Aberrant differentiation of neuromuscular junctions in mice lacking s-laminin/laminin beta 2. Nature, 1995. 374(6519): p. 258-62.

25. Li, D. Y., B. Brooke, E. C. Davis, R. P. Mecham, L. K. Sorensen, B. B. Boak, E. Eichwald, and M. T. Keating, Elastin is an essential determinant of arterial morphogenesis. Nature, 1998. 393(6682): p. 276-280.

26. Desai, N., P. Rambhia, and A. Gishto, Human embryonic stem cell cultivation: historical perspective and evolution of xeno-free culture systems. Reproductive Biology and Endocrinology : RB&E, 2015. 13: p. 9.

27. Ranieri, J. P., R. Bellamkonda, E. J. Bekos, T. G. Vargo, J. A. Gardella, Jr., and P. Aebischer, Neuronal cell attachment to fluorinated ethylene propylene films with covalently immobilized laminin oligopeptides YIGSR and IKVAV. II. J Biomed Mater Res, 1995. 29(6): p. 779-85.

28. Bellamkonda, R., J. P. Ranieri, and P. Aebischer, Laminin oligopeptide derivatized agarose gels allow three-dimensional neurite extension in vitro. J Neurosci Res, 1995. 41(4): p. 501-9.

29. Schense, J. C., J. Bloch, P. Aebischer, and J. A. Hubbell, Enzymatic incorporation of bioactive peptides into fibrin matrices enhances neurite extension. Nat Biotechnol, 2000. 18(4): p. 415-9.

30. Rodríguez-Cabello, J. C., S. Prieto, J. Reguera, F. J. Arias, and A. Ribeiro, Biofunctional design of elastin-like polymers for advanced applications in nanobiotechnology. J. Biomater. Sci. Polym. Ed., 2007. 18(3): p. 269-86.

31. McDaniel, J. R., J. A. Mackay, F. G. Quiroz, and A. Chilkoti, Recursive directional ligation by plasmid reconstruction allows rapid and seamless cloning of oligomeric genes. Biomacromolecules, 2010. 11(4): p. 944-52.

32. Herrero-Vanrell, R., A. C. Rincón, M. Alonso, V. Reboto, I. T. Molina-Martinez, and J. C. Rodríguez-Cabello, Self-assembled particles of an elastin-like polymer as vehicles for controlled drug release. J. Control. Release, 2005. 102(1): p. 113-22.

33. McDaniel, J. R., D. J. Callahan, and A. Chilkoti, Drug delivery to solid tumors by elastin-like polypeptides. Adv. Drug Deliv. Rev., 2010. 62(15): p. 1456-67.

34. Meyer, D. E., B. C. Shin, G. A. Kong, M. W. Dewhirst, and A. Chilkoti, Drug targeting using thermally responsive polymers and local hyperthermia. J. Control. Release, 2001. 74(1-3): p. 213-24.

35. Amruthwar, S. S. and A. V. Janorkar, In vitro evaluation of elastin-like polypeptide-collagen composite scaffold for bone tissue engineering. Dent. Mater., 2013. 29(2): p. 211-20.

36. Kinikoglu, B., J. C. Rodríguez-Cabello, O. Damour, and V. Hasirci, The influence of elastin-like recombinant polymer on the self-renewing potential of a 3D tissue equivalent derived from human lamina propria fibroblasts and oral epithelial cells. Biomaterials, 2011. 32(25): p. 5756-64.

37. Chow, D., M. L. Nunalee, D. W. Lim, A. J. Simnick, and A. Chilkoti, Peptide-based Biopolymers in Biomedicine and Biotechnology. Mater. Sci. Eng. R. Rep., 2008. 62(4): p. 125-155.

38. Mazia D, Schatten G, Sale W. Adhesion of cells to surfaces coated with polylysine. Applications to electron microscopy. The Journal of cell biology. 1975;66(1):198-200.

39. Ingrole, R. S., W. Tao, J. N. Tripathy, and H. S. Gill, Synthesis and Immunogenicity Assessment of Elastin-Like Polypeptide-M2e Construct as an Influenza Antigen. Nano Life, 2014. 4(2): p. 1450004.

40. Hassouneh, W., S. R. MacEwan, and A. Chilkoti, Fusions of elastin-like polypeptides to pharmaceutical proteins. Methods Enzymol, 2012. 502: p. 215-37.

41. Kam L, Shain W, Turner J N, Bizios R. Axonal outgrowth of hippocampal neurons on micro-scale networks of polylysine-conjugated laminin. Biomaterials. 2001. 22 p. 1049-1054

42. Sallach, R. E., W. Cui, F. Balderrama, A. W. Martinez, J. Wen, C. A. Haller, J. V. Taylor, E. R. Wright, R. C. Long, Jr., and E. L. Chaikof, Long-term biostability of self-assembling protein polymers in the absence of covalent crosslinking. Biomaterials, 2010. 31(4): p. 779-91.

43. Jeon, K., H. Lim, J.-H. Kim, D. Han, E.-R. Lee, G.-M. Yang, M.-K. Song, J.-H. Kim, and S.-G. Cho, Bax inhibitor-1 enhances survival and neuronal differentiation of embryonic stem cells via differential regulation of mitogen-activated protein kinases activities. Biochimica et Biophysica Acta (BBA)—Molecular Cell Research, 2012. 1823(12): p. 2190-2200.

44. Kwon, Y. W., Y. J. Chung, J. Kim, H. J. Lee, J. Park, T. Y. Roh, H. J. Cho, C. H. Yoon, B. K. Koo, and H. S. Kim, Comparative study of efficacy of dopaminergic neuron differentiation between embryonic stem cell and protein-based induced pluripotent stem cell. PLoS One, 2014. 9(1): p. e85736.

45. Chandel, N. S., H. Jasper, T. T. Ho, and E. Passegue, Metabolic regulation of stem cell function in tissue homeostasis and organismal ageing. Nat Cell Biol, 2016. 18(8): p. 823-32.

46. Burgess, R. J., M. Agathocleous, and S. J. Morrison, Metabolic regulation of stem cell function. J Intern Med, 2014. 276(1): p. 12-24.

47. Kim, D.-Y., I. Rhee, and J. Paik, Metabolic circuits in neural stem cells. Cellular and molecular life sciences : CMLS, 2014. 71(21): p. 4221-4241.

48. Teslaa, T. and M. A. Teitell, Pluripotent stem cell energy metabolism: an update. EMBO J, 2015. 34(2): p. 138-53.

49. Adil, M. M., G. M. C. Rodrigues, R. U. Kulkarni, A. T. Rao, N. E. Chernaysky, E. W. Miller, and D. V. Schaffer, Efficient generation of hPSC-derived midbrain dopaminergic neurons in a fully defined, scalable, 3D biomaterial platform. 2017. 7: p. 40573.

50. Stansley, B. J. and B. K. Yamamoto, 1-dopa-induced dopamine synthesis and oxidative stress in serotonergic cells. Neuropharmacology, 2013. 67: p. 243-251.

51. Hartfield, E. M., M. Yamasaki-Mann, H. J. Ribeiro Fernandes, J. Vowles, W. S. James, S. A. Cowley, and R. Wade-Martins, Physiological Characterisation of Human iPS-Derived Dopaminergic Neurons. PLOS ONE, 2014. 9(2): p. e87388.

52. Grace, A. A. and B. S. Bunney, The control of firing pattern in nigral dopamine neurons: burst firing. J Neurosci, 1984. 4(11): p. 2877-90.

53. Tieu, K., A Guide to Neurotoxic Animal Models of Parkinson's Disease. Cold Spring Harbor Perspectives in Medicine:, 2011. 1(1): p. a009316.

54. Kann, O. and R. Kovacs, Mitochondria and neuronal activity. Am J Physiol Cell Physiol, 2007. 292(2): p. C641-57.

55. Heo, J. Y., J. H. Park, S. J. Kim, K. S. Seo, J. S. Han, S. H. Lee, J. M. Kim, J. I. Park, S. K. Park, K. Lim, B. D. Hwang, M. Shong, and G. R. Kweon, D J-1 Null Dopaminergic Neuronal Cells Exhibit Defects in Mitochondrial Function and Structure: Involvement of Mitochondrial Complex I Assembly. PLOS ONE, 2012. 7(3): p. e32629.

56. Choi, S. W., A. A. Gerencser, D. W. Lee, S. Rajagopalan, D. G. Nicholls, J. K. Andersen, and M. D. Brand, Intrinsic Bioenergetic Properties and Stress Sensitivity of Dopaminergic Synaptosomes. The Journal of neuroscience : the official journal of the Society for Neuroscience, 2011. 31(12): p. 4524-4534.

57. Yin, X., M. Manczak, and P. H. Reddy, Mitochondria-targeted molecules MitoQ and SS31 reduce mutant huntingtin-induced mitochondrial toxicity and synaptic damage in Huntington's disease. Hum Mol Genet, 2016. 25(9): p. 1739-53.

58. Reddy, P. H., M. Manczak, X. Yin, M. C. Grady, A. Mitchell, R. Kandimalla, and C. S. Kuruva, Protective effects of a natural product, curcumin, against amyloid beta induced mitochondrial and synaptic toxicities in Alzheimer's disease. J Investig Med, 2016. 64(8): p. 1220-1234.

59. Manczak, M. and P. H. Reddy, Mitochondrial division inhibitor 1 protects against mutant huntingtin-induced abnormal mitochondrial dynamics and neuronal damage in Huntington's disease. Hum Mol Genet, 2015. 24(25): p. 7308-25.

60. Alvina, K., E. Tara, and K. Khodakhah, Developmental change in the contribution of voltage-gated Ca(2+) channels to the pacemaking of deep cerebellar nuclei neurons. Neuroscience, 2016. 322: p. 171-7.

61. Ben-Simon, Y., A. Rodenas-Ruano, K. Alvina, A. D. Lam, E. L. Stuenkel, P. E. Castillo, and U. Ashery, A Combined Optogenetic-Knockdown Strategy Reveals a Major Role of Tomosyn in Mossy Fiber Synaptic Plasticity. Cell Rep, 2015. 12(3): p. 396-404.

62. Alvina, K. and K. Khodakhah, KCa channels as therapeutic targets in episodic ataxia type-2. J Neurosci, 2010. 30(21): p. 7249-57.

63. Kriks, S., J. W. Shim, J. Piao, Y. M. Ganat, D. R. Wakeman, Z. Xie, L. Carrillo-Reid, G. Auyeung, C. Antonacci, A. Buch, L. Yang, M. F. Beal, D. J. Surmeier, J. H. Kordower, V. Tabar, and L. Studer, Dopamine neurons derived from human ES cells efficiently engraft in animal models of Parkinson's disease. Nature, 2011. 480(7378): p. 547-51.

64. Wernig, M., J. P. Zhao, J. Pruszak, E. Hedlund, D. Fu, F. Soldner, V. Broccoli, M. Constantine-Paton, O. Isacson, and R. Jaenisch, Neurons derived from reprogrammed fibroblasts functionally integrate into the fetal brain and improve symptoms of rats with Parkinson's disease. Proc Natl Acad Sci USA, 2008. 105(15): p. 5856-61.

65. Doi, D., B. Samata, M. Katsukawa, T. Kikuchi, A. Morizane, Y. Ono, K. Sekiguchi, M. Nakagawa, M. Parmar, and J. Takahashi, Isolation of human induced pluripotent stem cell-derived dopaminergic progenitors by cell sorting for successful transplantation. Stem Cell Reports, 2014. 2(3): p. 337-50.

### REFERENCES—EXAMPLE 2

[1] S. Yusuf, D. Wood, J. Ralston, K. S. Reddy, The World Heart Federation's vision for worldwide cardiovascular disease prevention, Lancet 386(9991) (2015) 399-402.

[2] A. P. Beltrami, L. Barlucchi, D. Torella, M. Baker, F. Limana, S. Chimenti, H. Kasahara, M. Rota, E. Musso, K. Urbanek, A. Leri, J. Kajstura, B. Nadal-Ginard, P. Anversa, Adult cardiac stem cells are multipotent and support myocardial regeneration, Cell 114(6) (2003) 763-76.

[3] X. Guo, Y. Bai, L. Zhang, B. Zhang, N. Zagidullin, K. Carvalho, Z. Du, B. Cai, Cardiomyocyte differentiation of mesenchymal stem cells from bone marrow: new regulators and its implications, Stem Cell Res Ther 9(1) (2018) 44.

[4] M. G. Iachininoto, S. Capodimonti, M. V. Podda, C. G. Valentini, M. Bianchi, A. M. Leone, L. Teofili, G. Leone, In vitro cardiomyocyte differentiation of umbilical cord blood cells: crucial role for c-kit(+) cells, Cytotherapy 17(11) (2015) 1627-37.

[5] P. Muller, H. Lemcke, R. David, Stem Cell Therapy in Heart Diseases—Cell Types, Mechanisms and Improvement Strategies, Cell Physiol Biochem 48(6) (2018) 2607-2655.

[6] R. L. Carpenedo, C. Y. Sargent, T. C. McDevitt, Rotary suspension culture enhances the efficiency, yield, and homogeneity of embryoid body differentiation, Stem Cells 25(9) (2007) 2224-34.

[7] A. T. Lynch, S. Mazzotta, S. Hoppler, Cardiomyocyte Differentiation from Mouse Embryonic Stem Cells, Methods Mol Biol 1816 (2018) 55-66.

[8] L. Yang, M. H. Soonpaa, E. D. Adler, T. K. Roepke, S. J. Kattman, M. Kennedy, E. Henckaerts, K. Bonham, G. W. Abbott, R. M. Linden, L. J. Field, G. M. Keller, Human cardiovascular progenitor cells develop from a KDR+ embryonic-stem-cell-derived population, Nature 453(7194) (2008) 524-8.

[9] R. Passier, D. W. Oostwaard, J. Snapper, J. Kloots, R. J. Hassink, E. Kuijk, B. Roelen, A. B. de la Riviere, C. Mummery, Increased cardiomyocyte differentiation from human embryonic stem cells in serum-free cultures, Stem Cells 23(6) (2005) 772-80.

[10] J. S. Aguilar, A. N. Begum, J. Alvarez, X. B. Zhang, Y. Hong, J. Hao, Directed cardiomyogenesis of human pluripotent stem cells by modulating Wnt/beta-catenin and BMP signalling with small molecules, Biochem J 469(2) (2015) 235-41.

[11] R. Peerani, B. M. Rao, C. Bauwens, T. Yin, G. A. Wood, A. Nagy, E. Kumacheva, P. W. Zandstra, Niche-mediated control of human embryonic stem cell self-renewal and differentiation, EMBO J 26(22) (2007) 4744-55.

[12] Y. S. Hwang, B. G. Chung, D. Ortmann, N. Hattori, H. C. Moeller, A. Khademhosseini, Microwell-mediated control of embryoid body size regulates embryonic stem cell fate via differential expression of WNT5a and WNT11, Proc Natl Acad Sci USA 106(40) (2009) 16978-83.

[13] J. Hescheler, B. K. Fleischmann, S. Lentini, V. A. Maltsev, J. Rohwedel, A. M. Wobus, K. Addicks, Embryonic stem cells: a model to study structural and functional properties in cardiomyogenesis, Cardiovasc Res 36(2) (1997) 149-62.

[14] A. Khademhosseini, L. Ferreira, J. Blumling, 3rd, J. Yeh, J. M. Karp, J. Fukuda, R. Langer, Co-culture of human embryonic stem cells with murine embryonic fibroblasts on microwell-patterned substrates, Biomaterials 27(36) (2006) 5968-77.

[15] M. Charnley, M. Textor, A. Khademhosseini, M. P. Lutolf, Integration column: microwell arrays for mammalian cell culture, Integr Biol (Camb) 1(11-12) (2009) 625-34.

[16] Y. Y. Choi, B. G. Chung, D. H. Lee, A. Khademhosseini, J. H. Kim, S. H. Lee, Controlled-size embryoid body formation in concave microwell arrays, Biomaterials 31(15) (2010) 4296-303.

[17] P. W. Burridge, E. Matsa, P. Shukla, Z. C. Lin, J. M. Churko, A. D. Ebert, F. Lan, S. Diecke, B. Huber, N. M. Mordwinkin, J. R. Plews, O. J. Abilez, B. Cui, J. D. Gold, J. C. Wu, Chemically defined generation of human cardiomyocytes, Nat Methods 11(8) (2014) 855-60.

[18] X. Lian, J. Zhang, S. M. Azarin, K. Zhu, L. B. Hazeltine, X. Bao, C. Hsiao, T. J. Kamp, S. P. Palecek, Directed cardiomyocyte differentiation from human pluripotent stem cells by modulating Wnt/beta-catenin signaling under fully defined conditions, Nat Protoc 8(1) (2013) 162-75.

[19] Y. Jiang, P. Park, S. M. Hong, K. Ban, Maturation of Cardiomyocytes Derived from Human Pluripotent Stem Cells: Current Strategies and Limitations, Mol Cells 41(7) (2018) 613-621.

[20] F. Gattazzo, A. Urciuolo, P. Bonaldo, Extracellular matrix: a dynamic microenvironment for stem cell niche, Biochim Biophys Acta 1840(8) (2014) 2506-19.

[21] M. Ahmed, C. Ffrench-Constant, Extracellular Matrix Regulation of Stem Cell Behavior, Curr Stem Cell Rep 2 (2016) 197-206.

[22] L. A. Flanagan, L. M. Rebaza, S. Derzic, P. H. Schwartz, E. S. Monuki, Regulation of human neural precursor cells by laminin and integrins, J Neurosci Res 83(5) (2006) 845-56.

[23] A. J. Engler, S. Sen, H. L. Sweeney, D. E. Discher, Matrix elasticity directs stem cell lineage specification, Cell 126(4) (2006) 677-89.

[24] J. Ozsvar, S. M. Mithieux, R. Wang, A. S. Weiss, Elastin-based biomaterials and mesenchymal stem cells, Biomater Sci 3(6) (2015) 800-9.

[25] J. Rosenbloom, W. R. Abrams, R. Mecham, Extracellular matrix 4: the elastic fiber, FASEB J 7(13) (1993) 1208-18.

[26] T. N. Thimm, J. M. Squirrell, Y. Liu, K. W. Eliceiri, B. M. Ogle, Endogenous Optical Signals Reveal Changes of Elastin and Collagen Organization During Differentiation of Mouse Embryonic Stem Cells, Tissue Eng Part C Methods 21(10) (2015) 995-1004.

[27] X. Tian, J. Fan, M. Yu, Y. Zhao, Y. Fang, S. Bai, W. Hou, H. Tong, Adipose stem cells promote smooth muscle cells to secrete elastin in rat abdominal aortic aneurysm, PLoS One 9(9) (2014) e108105.

[28] M. Wanjare, F. Kuo, S. Gerecht, Derivation and maturation of synthetic and contractile vascular smooth muscle cells from human pluripotent stem cells, Cardiovasc Res 97(2) (2013) 321-30.

[29] S. Jockenhoevel, G. Zund, S. P. Hoerstrup, K. Chalabi, J. S. Sachweh, L. Demircan, B. J. Messmer, M. Turina, Fibrin gel—advantages of a new scaffold in cardiovascular tissue engineering, Eur J Cardiothorac Surg 19(4) (2001) 424-30.

[30] Y. Pang, X. Wang, D. Lee, H. P. Greisler, Dynamic quantitative visualization of single cell alignment and migration and matrix remodeling in 3-D collagen hydrogels under mechanical force, Biomaterials 32(15) (2011) 3776-83.

[31] A. Marsano, R. Maidhof, J. Luo, K. Fujikara, E. E. Konofagou, A. Banfi, G. Vunjak-Novakovic, The effect of controlled expression of VEGF by transduced myoblasts in a cardiac patch on vascularization in a mouse model of myocardial infarction, Biomaterials 34(2) (2013) 393-401.

[32] R. Bolli, A. Chugh, J. Loughran, J. Kajstura, P. Anversa, Cardiac stem cells in patients with ischaemic cardiomyopathy—Authors' reply, Lancet 379(9819) (2012) 891-892.

[33] R. R. Makkar, R. R. Smith, K. Cheng, K. Malliaras, L. E. Thomson, D. Berman, L. S. Czer, L. Marban, A. Mendizabal, P. V. Johnston, S. D. Russell, K. H. Schuleri, A. C. Lardo, G. Gerstenblith, E. Marban, Intracoronary cardiosphere-derived cells for heart regeneration after myocardial infarction (CADUCEUS): a prospective, randomised phase 1 trial, Lancet 379(9819) (2012) 895-904.

[34] A. Behfar, R. Crespo-Diaz, A. Terzic, B. J. Gersh, Cell therapy for cardiac repair—lessons from clinical trials, Nat Rev Cardiol 11(4) (2014) 232-46.

[35] D. Hou, E. A. Youssef, T. J. Brinton, P. Zhang, P. Rogers, E. T. Price, A. C. Yeung, B. H. Johnstone, P. G. Yock, K. L. March, Radiolabeled cell distribution after intramyocardial, intracoronary, and interstitial retrograde coronary venous delivery: implications for current clinical trials, Circulation 112(9 Suppl) (2005) I150-6.

[36] K. Y. Ye, L. D. Black, 3rd, Strategies for tissue engineering cardiac constructs to affect functional repair following myocardial infarction, J Cardiovasc Transl Res 4(5) (2011) 575-91.

[37] R. Gaetani, G. Rizzitelli, I. Chimenti, L. Barile, E. Forte, V. Ionta, F. Angelini, J. P. Sluijter, A. Barbetta, E. Messina, G. Frati, Cardiospheres and tissue engineering for myocardial regeneration: potential for clinical application, J Cell Mol Med 14(5) (2010) 1071-7.

[38] M. B. van Eldijk, C. L. McGann, K. L. Kiick, J. C. van Hest, Elastomeric polypeptides, Top Curr Chem 310 (2012) 71-116.

[39] H. Betre, S. R. Ong, F. Guilak, A. Chilkoti, B. Fermor, L. A. Setton, Chondrocytic differentiation of human adipose-derived adult stem cells in elastin-like polypeptide, Biomaterials 27(1) (2006) 91-9.

[40] A. Paul, M. Stuhrenberg, S. Chen, D. Rhee, W. K. Lee, T. W. Odom, S. C. Heilshorn, A. Enejder, Micro- and nano-patterned elastin-like polypeptide hydrogels for stem cell culture, Soft Matter 13(34) (2017) 5665-5675.

[41] R. S. Ingrole, W. Tao, J. N. Tripathy, H. S. Gill, Synthesis and Immunogenicity Assessment of Elastin-Like Polypeptide-M2e Construct as an Influenza Antigen, Nano Life 4(2) (2014) 1450004.

[42] C. H. Lee, R. S. J . Ingrole, H. S. Gill, Generation of induced pluripotent stem cells using elastin like polypeptides as a non-viral gene delivery system, Biochim Biophys Acta Mol Basis Dis (2019).

[43] B. Leobon, J. Roncalli, C. Joffre, M. Mazo, M. Boisson, C. Barreau, D. Calise, E. Arnaud, M. Andre, M. Puceat, L. Penicaud, F. Prosper, V. Planat-Benard, L. Casteilla, Adipose-derived cardiomyogenic cells: in vitro expansion and functional improvement in a mouse model of myocardial infarction, Cardiovasc Res 83(4) (2009) 757-67.

[44] N. J. Palpant, S. Yasuda, O. MacDougald, J. M. Metzger, Non-canonical Wnt signaling enhances differentiation of Sca1+/c-kit+ adipose-derived murine stromal vascular cells into spontaneously beating cardiac myocytes, J Mol Cell Cardiol 43(3) (2007) 362-70.

[45] G. Yang, Z. Xiao, X. Ren, H. Long, K. Ma, H. Qian, Y. Guo, Obtaining spontaneously beating cardiomyocyte-like cells from adipose-derived stromal vascular fractions cultured on enzyme-crosslinked gelatin hydrogels, Sci Rep 7 (2017) 41781.

[46] M. E. Strek, F. S. Williams, G. J. Gleich, A. R. Leff, S. R. White, Mechanisms of smooth muscle contraction elicited by cationic proteins in guinea pig trachealis, Am J Physiol 270(1 Pt 1) (1996) L133-40.

[47] M. A. Laflamme, K. Y. Chen, A. V. Naumova, V. Muskheli, J. A. Fugate, S. K. Dupras, H. Reinecke, C. Xu, M. Hassanipour, S. Police, C. O'Sullivan, L. Collins, Y. Chen, E. Minami, E. A. Gill, S. Ueno, C. Yuan, J. Gold, C. E. Murry, Cardiomyocytes derived from human embryonic stem cells in pro-survival factors enhance function of infarcted rat hearts, Nat Biotechnol 25(9) (2007) 1015-24.

### REFERENCES—EXAMPLE 3

[1] E. J. Benjamin, P. Muntner, A. Alonso, M. S. Bittencourt, C. W. Callaway, A. P. Carson, A. M. Chamberlain, A. R. Chang, S. Cheng, S. R. Das, F. N. Delling, L. Djousse, M. S. V. Elkind, J. F. Ferguson, M. Fornage, L. C. Jordan, S. S. Khan, B. M. Kissela, K. L. Knutson, T. W. Kwan, D. T. Lackland, T. T. Lewis, J. H. Lichtman, C. T. Longenecker, M. S. Loop, P. L. Lutsey, S. S. Martin, K. Matsushita, A. E. Moran, M. E. Mussolino, M. O'Flaherty, A. Pandey, A. M. Perak, W. D. Rosamond, G. A. Roth, U. K. A. Sampson, G. M. Satou, E. B. Schroeder, S. H. Shah, N. L. Spartano, A. Stokes, D. L. Tirschwell, C. W. Tsao, M. P. Turakhia, L. B. VanWagner, J. T. Wilkins, S. S. Wong, S. S. Virani, Heart Disease and Stroke Statistics-2019 Update: A Report From the American Heart Association, Circulation 139(10) (2019) e56-e528.

[2] M. N. Banerjee, R. Bolli, J. M. Hare, Clinical Studies of Cell Therapy in Cardiovascular Medicine: Recent Developments and Future Directions, Circ Res 123(2) (2018) 266-287.

[3] P. Menasche, Cell therapy trials for heart regeneration—lessons learned and future directions, Nat Rev Cardiol 15(11) (2018) 659-671.

[4] S. J. Kattman, A. D. Witty, M. Gagliardi, N. C. Dubois, M. Niapour, A. Hotta, J. Ellis, G. Keller, Stage-specific optimization of activin/nodal and BMP signaling promotes cardiac differentiation of mouse and human pluripotent stem cell lines, Cell Stem Cell 8(2) (2011) 228-40.

[5] R. L. Carpenedo, C. Y. Sargent, T. C. McDevitt, Rotary suspension culture enhances the efficiency, yield, and homogeneity of embryoid body differentiation, Stem Cells 25(9) (2007) 2224-34.

[6] A. T. Lynch, S. Mazzotta, S. Hoppler, Cardiomyocyte Differentiation from Mouse Embryonic Stem Cells, Methods Mol Biol 1816 (2018) 55-66.

[7] L. Yang, M. H. Soonpaa, E. D. Adler, T. K. Roepke, S. J. Kattman, M. Kennedy, E. Henckaerts, K. Bonham, G. W. Abbott, R. M. Linden, L. J. Field, G. M. Keller, Human cardiovascular progenitor cells develop from a KDR+ embryonic-stem-cell-derived population, Nature 453(7194) (2008) 524-8.

[8] R. Passier, D. W. Oostwaard, J. Snapper, J. Kloots, R. J. Hassink, E. Kuijk, B. Roelen, A. B. de la Riviere, C. Mummery, Increased cardiomyocyte differentiation from human embryonic stem cells in serum-free cultures, Stem Cells 23(6) (2005) 772-80.

[9] S. Bhattacharya, P. W. Burridge, E. M. Kropp, S. L. Chuppa, W. M. Kwok, J. C. Wu, K. R. Boheler, R. L. Gundry, High efficiency differentiation of human pluripotent stem cells to cardiomyocytes and characterization by flow cytometry, J Vis Exp (91) (2014) 52010.

[10] A. Leitolis, A. W. Robert, I. T. Pereira, A. Correa, M. A. Stimamiglio, Cardiomyogenesis Modeling Using Pluripotent Stem Cells: The Role of Microenvironmental Signaling, Frontiers in Cell and Developmental Biology 7(164) (2019).

[11] X. Yang, L. Pabon, C. E. Murry, Engineering adolescence: maturation of human pluripotent stem cell-derived cardiomyocytes, Circ Res 114(3) (2014) 511-23.

[12] E. Karbassi, A. Fenix, S. Marchiano, N. Muraoka, K. Nakamura, X. Yang, C. E. Murry, Cardiomyocyte maturation: advances in knowledge and implications for regenerative medicine, Nat Rev Cardiol (2020).

[13] G. J. Scuderi, J. Butcher, Naturally Engineered Maturation of Cardiomyocytes, Front Cell Dev Biol 5 (2017) 50.

[14] F. Gattazzo, A. Urciuolo, P. Bonaldo, Extracellular matrix: a dynamic microenvironment for stem cell niche, Biochim Biophys Acta 1840(8) (2014) 2506-19.

[15] M. Ahmed, C. Ffrench-Constant, Extracellular Matrix Regulation of Stem Cell Behavior, Curr Stem Cell Rep 2 (2016) 197-206.

[16] L. A. Flanagan, L. M. Rebaza, S. Derzic, P. H. Schwartz, E. S. Monuki, Regulation of human neural precursor cells by laminin and integrins, J Neurosci Res 83(5) (2006) 845-56.

[17] A. J. Engler, S. Sen, H. L. Sweeney, D. E. Discher, Matrix elasticity directs stem cell lineage specification, Cell 126(4) (2006) 677-89.

[18] J. Ozsvar, S. M. Mithieux, R. Wang, A. S. Weiss, Elastin-based biomaterials and mesenchymal stem cells, Biomater Sci 3(6) (2015) 800-9.

[19] J. Rosenbloom, W. R. Abrams, R. Mecham, Extracellular matrix 4: the elastic fiber, FASEB J 7(13) (1993) 1208-18.

[20] T. N. Thimm, J. M. Squirrell, Y. Liu, K. W. Eliceiri, B. M. Ogle, Endogenous Optical Signals Reveal Changes of Elastin and Collagen Organization During Differentiation of Mouse Embryonic Stem Cells, Tissue Eng Part C Methods 21(10) (2015) 995-1004.

[21] X. Tian, J. Fan, M. Yu, Y. Zhao, Y. Fang, S. Bai, W. Hou, H. Tong, Adipose stem cells promote smooth muscle cells to secrete elastin in rat abdominal aortic aneurysm, PLoS One 9(9) (2014) e108105.

[22] M. Wanjare, F. Kuo, S. Gerecht, Derivation and maturation of synthetic and contractile vascular smooth muscle cells from human pluripotent stem cells, Cardiovasc Res 97(2) (2013) 321-30.

[23] S. Jockenhoevel, G. Zund, S. P. Hoerstrup, K. Chalabi, J. S. Sachweh, L. Demircan, B. J. Messmer, M. Turina, Fibrin gel—advantages of a new scaffold in cardiovascular tissue engineering, Eur J Cardiothorac Surg 19(4) (2001) 424-30.

[24] Y. Pang, X. Wang, D. Lee, H. P. Greisler, Dynamic quantitative visualization of single cell alignment and migration and matrix remodeling in 3-D collagen hydrogels under mechanical force, Biomaterials 32(15) (2011) 3776-83.

[25] A. Marsano, R. Maidhof, J. Luo, K. Fujikara, E. E. Konofagou, A. Banfi, G. Vunjak-Novakovic, The effect of controlled expression of VEGF by transduced myoblasts in a cardiac patch on vascularization in a mouse model of myocardial infarction, Biomaterials 34(2) (2013) 393-401.

[26] D. W. Urry, T. L. Trapane, K. U. Prasad, Phase-structure transitions of the elastin polypentapeptide-water system within the framework of composition-temperature studies, Biopolymers 24(12) (1985) 2345-56.

[27] J. R. McDaniel, D. C. Radford, A. Chilkoti, A unified model for de novo design of elastin-like polypeptides with tunable inverse transition temperatures, Biomacromolecules 14(8) (2013) 2866-72.

[28] T. Christensen, W. Hassouneh, K. Trabbic-Carlson, A. Chilkoti, Predicting transition temperatures of elastin-like polypeptide fusion proteins, Biomacromolecules 14(5) (2013) 1514-9.

[29] H. Betre, S. R. Ong, F. Guilak, A. Chilkoti, B. Fermor, L. A. Setton, Chondrocytic differentiation of human adipose-derived adult stem cells in elastin-like polypeptide, Biomaterials 27(1) (2006) 91-9.

[30] A. Paul, M. Stuhrenberg, S. Chen, D. Rhee, W. K. Lee, T. W. Odom, S. C. Heilshorn, A. Enejder, Micro- and nano-patterned elastin-like polypeptide hydrogels for stem cell culture, Soft Matter 13(34) (2017) 5665-5675.

[31] E. Jin, P. T. Lee, W. B. Jeon, W.-J. Li, Effects of Elastin-Like Peptide on Regulation of Human Mesenchymal Stem Cell Behavior, Regenerative Engineering and Translational Medicine 2(2) (2016) 85-97.

[32] J. R. McDaniel, J. A. Mackay, F. G. Quiroz, A. Chilkoti, Recursive directional ligation by plasmid reconstruction allows rapid and seamless cloning of oligomeric genes, Biomacromolecules 11(4) (2010) 944-952.

[33] C. H. Lee, R. S. J. Ingrole, H. S. Gill, Generation of induced pluripotent stem cells using elastin like polypeptides as a non-viral gene delivery system, Biochimica et Biophysica Acta (BBA)—Molecular Basis of Disease 1866(4) (2020) 165405.

[34] R. S. Ingrole, W. Tao, J. N. Tripathy, H. S. Gill, Synthesis and Immunogenicity Assessment of Elastin-Like Polypeptide-M2e Construct as an Influenza Antigen, Nano Life 4(2) (2014) 1450004.

[35] 0. Sirenko, C. Crittenden, N. Callamaras, J. Hesley, Y. W. Chen, C. Funes, I. Rusyn, B. Anson, E. F. Cromwell, Multiparameter in vitro assessment of compound effects on cardiomyocyte physiology using iPSC cells, J Biomol Screen 18(1) (2013) 39-53.

[36] W. Huang da, B. T. Sherman, R. A. Lempicki, Systematic and integrative analysis of large gene lists using DAVID bioinformatics resources, Nat Protoc 4(1) (2009) 44-57.

[37] K. G. Chen, B. S. Mallon, R. S. Hamilton, O. A. Kozhich, K. Park, D. J. Hoeppner, P. G. Robey, R. D. McKay, Non-colony type monolayer culture of human embryonic stem cells, Stem Cell Res 9(3) (2012) 237-48.

[38] P. W. Burridge, E. Matsa, P. Shukla, Z. C. Lin, J. M. Churko, A. D. Ebert, F. Lan, S. Diecke, B. Huber, N. M. Mordwinkin, J. R. Plews, O. J. Abilez, B. Cui, J. D. Gold, J. C. Wu, Chemically defined generation of human cardiomyocytes, Nat Methods 11(8) (2014) 855-60.

[39] N. Cao, Z. Liu, Z. Chen, J. Wang, T. Chen, X. Zhao, Y. Ma, L. Qin, J. Kang, B. Wei, L. Wang, Y. Jin, H. T. Yang, Ascorbic acid enhances the cardiac differentiation of induced pluripotent stem cells through promoting the proliferation of cardiac progenitor cells, Cell Res 22(1) (2012) 219-36.

[40] S. Higuchi, Q. Lin, J. Wang, T. K. Lim, S. B. Joshi, G. S. Anand, M. C. Chung, M. P. Sheetz, H. Fujita, Heart extracellular matrix supports cardiomyocyte differentiation of mouse embryonic stem cells, J Biosci Bioeng 115(3) (2013) 320-5.

[41] A. M. Lompre, B. Nadal-Ginard, V. Mandavi, Expression of the cardiac ventricular alpha-and beta-myosin heavy chain genes is developmentally and hormonally regulated, J Biol Chem 259(10) (1984) 6437-46.

[42] M. K. McHale, L. A. Setton, A. Chilkoti, Synthesis and in vitro evaluation of enzymatically cross-linked elastin-like polypeptide gels for cartilaginous tissue repair, Tissue Eng 11(11-12) (2005) 1768-79.

[43] S. J. Kattman, T. L. Huber, G. M. Keller, Multipotent flk-1+ cardiovascular progenitor cells give rise to the cardiomyocyte, endothelial, and vascular smooth muscle lineages, Dev Cell 11(5) (2006) 723-32.

[44] J. S. Aguilar, A. N. Begum, J. Alvarez, X. B. Zhang, Y. Hong, J. Hao, Directed cardiomyogenesis of human pluripotent stem cells by modulating Wnt/beta-catenin and BMP signalling with small molecules, Biochem J 469(2) (2015) 235-41.

[45] M. A. Laflamme, K. Y. Chen, A. V. Naumova, V. Muskheli, J. A. Fugate, S. K. Dupras, H. Reinecke, C. Xu, M. Hassanipour, S. Police, C. O'Sullivan, L. Collins, Y. Chen, E. Minami, E. A. Gill, S. Ueno, C. Yuan, J. Gold, C. E. Murry, Cardiomyocytes derived from human embryonic stem cells in pro-survival factors enhance function of infarcted rat hearts, Nat Biotechnol 25(9) (2007) 1015-24.

[46] H. Hara, N. Takeda, M. Kondo, M. Kubota, T. Saito, J. Maruyama, T. Fujiwara, S. Maemura, M. Ito, A. T. Naito, M. Harada, H. Toko, S. Nomura, H. Kumagai, Y. Ikeda, H. Ueno, E. Takimoto, H. Akazawa, H. Morita, H. Aburatani, Y. Hata, M. Uchiyama, I. Komuro, Discovery of a Small Molecule to Increase Cardiomyocytes and Protect the Heart After Ischemic Injury, JACC Basic Transl Sci 3(5) (2018) 639-653.

[47] S. Yoshida, S. Miyagawa, S. Fukushima, T. Kawamura, N. Kashiyama, F. Ohashi, T. Toyofuku, K. Toda, Y. Sawa, Maturation of Human Induced Pluripotent Stem Cell-Derived Cardiomyocytes by Soluble Factors from Human Mesenchymal Stem Cells, Mol Ther 26(11) (2018) 2681-2695.

[48] K. L. Coulombe, V. K. Bajpai, S. T. Andreadis, C. E. Murry, Heart regeneration with engineered myocardial tissue, Annu Rev Biomed Eng 16 (2014) 1-28.

[49] L. Ye, Y. H. Chang, Q. Xiong, P. Zhang, L. Zhang, P. Somasundaram, M. Lepley, C. Swingen, L. Su, J. S. Wendel, J. Guo, A. Jang, D. Rosenbush, L. Greder, J. R. Dutton, J. Zhang, T. J. Kamp, D. S. Kaufman, Y. Ge, J. Zhang, Cardiac repair in a porcine model of acute myocardial infarction with human induced pluripotent stem cell-derived cardiovascular cells, Cell Stem Cell 15(6) (2014) 750-61.

[50] A. Khademhosseini, L. Ferreira, J. Blumling, 3rd, J. Yeh, J. M. Karp, J. Fukuda, R. Langer, Co-culture of human embryonic stem cells with murine embryonic fibroblasts on microwell-patterned substrates, Biomaterials 27(36) (2006) 5968-77.

[51] L. C. Stevens, Embryonic potency of embryoid bodies derived from a transplantable testicular teratoma of the mouse, Dev Biol 2 (1960) 285-97.

[52] C. Xu, S. Police, N. Rao, M. K. Carpenter, Characterization and enrichment of cardiomyocytes derived from human embryonic stem cells, Circ Res 91(6) (2002) 501-8.

[53] P. W. Burridge, D. Anderson, H. Priddle, M. D. Barbadillo Munoz, S. Chamberlain, C. Allegrucci, L. E. Young, C. Denning, Improved human embryonic stem cell embryoid body homogeneity and cardiomyocyte differentiation from a novel V-96 plate aggregation system highlights interline variability, Stem Cells 25(4) (2007) 929-38.

[54] B. Leobon, J. Roncalli, C. Joffre, M. Mazo, M. Boisson, C. Barreau, D. Calise, E. Arnaud, M. Andre, M. Puceat, L. Penicaud, F. Prosper, V. Planat-Benard, L. Casteilla, Adipose-derived cardiomyogenic cells: in vitro expansion and functional improvement in a mouse model of myocardial infarction, Cardiovasc Res 83(4) (2009) 757-67.

[55] N. J. Palpant, S. Yasuda, O. MacDougald, J. M. Metzger, Non-canonical Wnt signaling enhances differentiation of Sca1+/c-kit+ adipose-derived murine stromal vascular cells into spontaneously beating cardiac myocytes, J Mol Cell Cardiol 43(3) (2007) 362-70.

[56] G. Yang, Z. Xiao, X. Ren, H. Long, K. Ma, H. Qian, Y. Guo, Obtaining spontaneously beating cardiomyocyte-like cells from adipose-derived stromal vascular fractions cultured on enzyme-crosslinked gelatin hydrogels, Sci Rep 7 (2017) 41781.

[57] R. Santoro, G. L. Perrucci, A. Gowran, G. Pompilio, Unchain My Heart: Integrins at the Basis of iPSC Cardiomyocyte Differentiation, Stem Cells Int 2019 (2019) 8203950.

[58] M. C. Daley, S. L. Fenn, L. D. Black, 3rd, Applications of Cardiac Extracellular Matrix in Tissue Engineering and Regenerative Medicine, Adv Exp Med Biol 1098 (2018) 59-83.

[59] P. Sreejit, R. S. Verma, Natural ECM as biomaterial for scaffold based cardiac regeneration using adult bone marrow derived stem cells, Stem Cell Rev Rep 9(2) (2013) 158-71.

[60] C. Correia, M. Serra, N. Espinha, M. Sousa, C. Brito, K. Burkert, Y. Zheng, J. Hescheler, M. J. Carrondo, T. Saric, P. M. Alves, Combining hypoxia and bioreactor hydrodynamics boosts induced pluripotent stem cell differentiation towards cardiomyocytes, Stem Cell Rev Rep 10(6) (2014) 786-801.

[61] L. van den Brink, C. Grandela, C. L. Mummery, R. P. Davis, Inherited cardiac diseases, pluripotent stem cells, and genome editing combined-the past, present, and future, Stem Cells 38(2) (2020) 174-186.

[62] E. Tejeda-Montes, K. H. Smith, M. Poch, M. J. Lopez-Bosque, L. Martin, M. Alonso, E. Engel, A. Mata, Engineering membrane scaffolds with both physical and biomolecular signaling, Acta Biomater 8(3) (2012) 998-1009.

[63] G. Benton, I. Arnaoutova, J. George, H. K. Kleinman, J. Koblinski, Matrigel: from discovery and ECM mimicry to assays and models for cancer research, Adv Drug Deliv Rev 79-80 (2014) 3-18.

What is claimed is:

1. A polypeptide for use in a three dimensional (3D) culture system for the growth of cells comprising:
one or more repeats of a sequence n₁-(X₁X₂GXP)-n₂ (SEQ ID NO:8), wherein X, X₁, X₂ are any amino acid, wherein X, X₁, and X₂ can be the same or different amino acid, wherein n₁ and n₂ are equal to or greater than one.

2. The polypeptide of claim 1, the polypeptide has the sequence selected from at least one of [(X₁X₂GXP)(X₃X₄GXP)]ₙ₁, (SEQ ID NO:68) [(X₁X₂GXP)ₙ₁(X₃X₄GXP)ₙ₂] (SEQ ID NO:69); (12) [(X₁X₂GXP)ₙ₁(X₃X₄GXP)ₙ₂(X₁X₂GXP)ₙ₁] (SEQ ID NO:70), wherein X, X₁, X₂, X₃, and X₄ are any amino acid, and n₁ and n₂ are greater than or equal to one; wherein X₁ and X₂ can be the same or different from each other, and X₃ and X₄ can be the same or different from each other; or wherein at least one of X₁ or X₂ is different from X₃ or X₄, or wherein X is valine, or X₁=G, X₂=Y and A (in 1:4 ratio) and X=V.

3. The polypeptide of claim 1, further comprising attaching to, or forming a fusion protein with, the polypeptide and at least a portion of an extracellular matrix component selected from at least one of: glycosaminoglycans (GAGs), proteoglycans, and/or proteins such as but not limited to laminin, fibronectin, vitronectin, collagen, elastin, fibrillin, fibulin, tenascin, perlecan, versican, aggrecan, neurocan, brevican, keratan, hyaluronic acid, heparan, or chondroitin, and wherein the fusion protein can be at an amino, a carboxy, or both the amino and carboxy ends of the polypeptide;
attaching to, or forming a fusion protein with, the polypeptide and at least a portion of a growth factor or cytokine selected from at least one of: leukemia inhibitory factor, insulin, insulin like growth factors, epidermal growth factor, fibroblast growth factors including basic fibroblast growth factor, vascular endothelial growth factor, transforming growth factor-β, platelet-derived growth factor, neurotrophic factors, interleukin-2, stem cell factor, Fms-like tyrosine kinase 3/fetal liver kinase-2, granulocyte-macrophage colony-stimulating factor, interleukin 1 alpha, or granulocyte colony-stimulating factor, and wherein the fusion protein can be at an amino, a carboxy, or both the amino and carboxy ends of the polypeptide; or
attaching to, or forming a fusion protein with, the polypeptide and at least a portion of a growth factor or cytokine and an extracellular matrix component, wherein the fusion proteins can be at an amino, a carboxy, or both the amino and carboxy ends of the polypeptide.

4. The polypeptide of claim 1, wherein the polypeptide is at least one of: provided in solution, attached to a substrate, or both; or the polypeptide is a fusion protein with an amino-terminal end that comprises a laminin domain and a carboxy-terminal end comprises an elastin domain; or
the polypeptide comprises at least one of: (1) a laminin domain comprising one or more VGKKKKKKKKG motifs (SEQ ID NO: 3); (2) one or more YIGSRVGKKKKKKKKG motifs (SEQ ID NO: 6); (3) one or more RNAIAEIIKDI motifs (SEQ ID NO: 2); (4) an elastin domain comprising one or more [(GAGVP)₂(GYGVP)(GAGVP)₂]₁₂ motifs (SEQ ID NO: 4); (5) [(GAGVP)₂(GYGVP)(GAGVP)₂]₂₄ motifs (SEQ ID NO: 5); (6) SEQ ID NOS: 1 and 4, (7) SEQ ID NOS: 1 and 5; (8) SEQ ID NOS: 3 and 4; (9) SEQ ID NOS: 3 and 5; (10) SEQ ID NOS: 2 and 4, or (6) SEQ ID NOS: 2 and 5; (11) any combination of SEQ ID NOS: 1, 2, 3, 4, or 5, wherein the polypeptide has the sequence selected from at least one of [(X₁X₂GXP)(X₃X₄GXP)]ₙ₁ (SEQ ID NO:68), [(X₁X₂GXP)ₙ₁(X₃X₄GXP)ₙ₂] (SEQ ID NO:69); (12) [(X₁X₂GXP)ₙ₁(X₃X₄GXP)ₙ₂(X₁X₂GXP)ₙ₁] (SEQ ID NO:70), wherein X₁, X₂, X₃, X₄, and X are any amino acid except proline; (13) [(X₁X₂GXP)(X₃X₄GXP)]ₙ₁ (SEQ ID NO:71), [(X₁X₂GXP)ₙ₁(X₃X₄GXP)ₙ₂] (SEQ ID NO:72); or (14) [(X₁X₂GXP)ₙ₁(X₃X₄GXP)ₙ₂(X₁X₂GXP)ₙ₁] (SEQ ID NO:73), wherein X₁, X₂, X₃, X₄ is any amino acid and X is an aliphatic amino acid.

5. A nucleic acid that encodes a polypeptide that comprises one or more repeats of a sequence n₁-(X₁X₂GXP)-n₂ (SEQ ID NO:8), wherein X, X₁, X₂ are any amino acid, wherein X, X₁, and X₂ can be the same or different amino acid, wherein n₁ and n₂ are equal to or greater than one.

6. The nucleic acid of claim 5, the polypeptide has the sequence selected from at least one of [(X₁X₂GXP)(X₃X₄GXP)]ₙ₁ (SEQ ID NO:68), [(X₁X₂GXP)ₙ₁(X₃X₄GXP)ₙ₂] (SEQ ID NO:69); (12) [(X₁X₂GXP)ₙ₁(X₃X₄GXP)ₙ₂(X₁X₂GXP)ₙ₁] (SEQ ID NO:70), wherein X, X₁, X₂, X₃, and X₄ are any amino acid, and n₁ and n₂ are greater than or equal to one; wherein X₁ and X₂ can be the same or different from each other, and X₃ and X₄ can be the same or different from each other; or wherein at least one of X₁ or X₂ is different from X₃ or X₄, or wherein X is valine, or X₁=G, X₂=Y and A (in 1:4 ratio) and X=V.

7. The nucleic acid of claim 5, further comprising attaching to, or forming a fusion protein with, the polypeptide and at least a portion of an extracellular matrix component selected from at least one of: glycosaminoglycans (GAGs), proteoglycans, and/or proteins such as but not limited to laminin, fibronectin, vitronectin, collagen, elastin, fibrillin, fibulin, tenascin, perlecan, versican, aggrecan, neurocan, brevican, keratan, hyaluronic acid, heparan, or chondroitin, and wherein the fusion protein can be at an amino, a carboxy, or both the amino and carboxy ends of the polypeptide;
attaching to, or forming a fusion protein with, the polypeptide and at least a portion of a growth factor or cytokine selected from at least one of: leukemia inhibitory factor, insulin, insulin like growth factors, epidermal growth factor, fibroblast growth factors including basic fibroblast growth factor, vascular endothelial growth factor, transforming growth factor-β, platelet-derived growth factor, neurotrophic factors, interleukin-2, stem cell factor, Fms-like tyrosine kinase 3/fetal liver kinase-2, granulocyte-macrophage colony-stimulating factor, interleukin 1 alpha, or granulocyte colony-stimulating factor, and wherein the fusion protein can be at an amino, a carboxy, or both the amino and carboxy ends of the polypeptide;
attaching to, or forming a fusion protein with, the polypeptide and at least a portion of a growth factor or cytokine and an extracellular matrix component, wherein the fusion proteins can be at an amino, a carboxy, or both the amino and carboxy ends of the polypeptide; or the polypeptide is a fusion protein with an amino-terminal end that comprises a laminin domain and a carboxy-terminal end comprises an elastin domain.

8. The nucleic acid of claim 5, wherein the polypeptide comprises at least one of: (1) a laminin domain comprising one or more VGKKKKKKKKG motifs (SEQ ID NO: 3); (2) one or more YIGSRVGKKKKKKKKG motifs (SEQ ID NO: 6); (3) one or more RNAIAEIIKDI motifs (SEQ ID NO: 2); (4) an elastin domain comprising one or more [(GAGVP)₂(GYGVP)(GAGVP)₂]₁₂ motifs (SEQ ID NO: 4); (5) [(GAGVP)₂(GYGVP)(GAGVP)₂]₂₄ motifs (SEQ ID NO: 5); (6) SEQ ID NOS: 1 and 4, (7) SEQ ID NOS: 1 and 5; (8) SEQ ID NOS: 3 and 4; (9) SEQ ID NOS: 3 and 5; (10) SEQ ID NOS: 2 and 4, or (6) SEQ ID NOS: 2 and 5; (11) any combination of SEQ ID NOS: 1, 2, 3, 4, or 5, wherein the polypeptide has the sequence selected from at least one of [(X₁X₂GXP)(X₃X₄GXP)]ₙ₁, [(X₁X₂GXP)ₙ₁(X₃X₄GXP)ₙ₂]; (12) [(X₁X₂GXP)ₙ₁(X₃X₄GXP)ₙ₂(X₁X₂GXP)ₙ₁], wherein X₁, X₂, X₃, X₄, and X are any amino acid except proline; (13) [(X₁X₂GXP)(X₃X₄GXP)]ₙ₁, [(X₁X₂GXP)ₙ₁(X₃X₄GXP)ₙ₂]; or (14) [(X₁X₂GXP)ₙ₁(X₃X₄GXP)ₙ₂(X₁X₂GXP)ₙ₁], wherein X₁, X₂, X₃, X₄ is any amino acid and X is an aliphatic amino acid.

9. A nucleic acid vector that encodes a polypeptide that comprises one or more repeats of a sequence n₁-(X₁X₂GXP)-n₂ (SEQ ID NO:8), wherein X, X₁, X₂ are any amino acid, wherein X, X₁, and X₂ can be the same or different amino acid, wherein n₁ and n₂ are equal to or greater than one; or the polypeptide has the sequence selected from at least one of [(X₁X₂GXP)(X₃X₄GXP)]ₙ₁, [(X₁X₂GXP)ₙ₁(X₃X₄GXP)ₙ₂], or [(X₁X₂GXP)ₙ₁(X₃X₄GXP)ₙ₂(X₁X₂GXP)ₙ₁], wherein X, X₁, X₂, X₃, and X₄ are any amino acid, and n₁ and n₂ are greater than or equal to one; wherein X₁ and X₂ can be the same or different from each other, and X₃ and X₄ can be the same or different from each other; or wherein at least one of X₁ or X₂ is different from X₃ or X₄, or wherein X is valine, or X₁=G, X₂=Y and A (in 1:4 ratio) and X=V.

10. The nucleic acid vector of claim 9, further comprising attaching to, or forming a fusion protein with, the polypeptide and at least a portion of an extracellular matrix component selected from at least one of: glycosaminoglycans (GAGs), proteoglycans, and/or proteins such as but not limited to laminin, fibronectin, vitronectin, collagen, elastin, fibrillin, fibulin, tenascin, perlecan, versican, aggrecan, neurocan, brevican, keratan, hyaluronic acid, heparan, or chondroitin, and wherein the fusion protein can be at an amino, a carboxy, or both the amino and carboxy ends of the polypeptide;
attaching to, or forming a fusion protein with, the polypeptide and at least a portion of a growth factor or cytokine selected from at least one of: leukemia inhibitory factor, insulin, insulin like growth factors, epidermal growth factor, fibroblast growth factors including basic fibroblast growth factor, vascular endothelial growth factor, transforming growth factor-β, platelet-derived growth factor, neurotrophic factors, interleukin-2, stem cell factor, Fms-like tyrosine kinase 3/fetal liver kinase-2, granulocyte-macrophage colony-stimulating factor, interleukin 1 alpha, or granulocyte colony-stimulating factor, and wherein the fusion protein can be at an amino, a carboxy, or both the amino and carboxy ends of the polypeptide; or
attaching to, or forming a fusion protein with, the polypeptide and at least a portion of a growth factor or cytokine and an extracellular matrix component, wherein the fusion proteins can be at an amino, a carboxy, or both the amino and carboxy ends of the polypeptide;
the polypeptide is provided in solution, attached to a substrate, or both; or
the polypeptide comprises at least one of: (1) a laminin domain comprising one or more VGKKKKKKKKG motifs (SEQ ID NO: 3); (2) one or more YIGSRVGKKKKKKKKG motifs (SEQ ID NO: 6); (3) one or more RNAIAEIIKDI motifs (SEQ ID NO: 2); (4) an elastin domain comprising one or more [(GAGVP)₂(GYGVP)(GAGVP)₂]₁₂ motifs (SEQ ID NO: 4); (5) [(GAGVP)₂(GYGVP)(GAGVP)₂]₂₄ motifs (SEQ ID NO: 5); (6) SEQ ID NOS: 1 and 4, (7) SEQ ID NOS: 1 and 5; (8) SEQ ID NOS: 3 and 4; (9) SEQ ID NOS: 3 and 5; (10) SEQ ID NOS: 2 and 4, or (6) SEQ ID NOS: 2 and 5; (11) any combination of SEQ ID NOS: 1, 2, 3, 4, or 5, wherein the polypeptide has the sequence selected from at least one of [(X₁X₂GXP)(X₃X₄GXP)]ₙ₁, [(X₁X₂GXP)ₙ₁(X₃X₄GXP)ₙ₂]; (12) [(X₁X₂GXP)ₙ₁(X₃X₄GXP)ₙ₂(X₁X₂GXP)ₙ₁], wherein X₁, X₂, X₃, X₄, and X are any amino acid except proline; (13) [(X₁X₂GXP)(X₃X₄GXP)]ₙ₁, [(X₁X₂GXP)ₙ₁(X₃X₄GXP)ₙ₂]; or (14) [(X₁X₂GXP)ₙ₁(X₃X₄GXP)ₙ₂(X₁X₂GXP)ₙ₁], wherein X₁, X₂, X₃, X₄ is any amino acid and X is an aliphatic amino acid.

11. A host cell that comprises a nucleic acid vector that encodes a polypeptide that comprises one or more repeats of a sequence n₁-(X₁X₂GXP)-n₂ (SEQ ID NO:8), wherein X, X₁, X₂ are any amino acid, wherein X, X₁, and X₂ can be the same or different amino acid, wherein n₁ and n₂ are equal to or greater than one, and optionally the host cell expresses or secretes the polypeptide.

12. A method of making a fusion protein comprising:
providing a host cell with a nucleic acid vector that expresses a polypeptide that comprises one or more repeats of a sequence n₁-(X₁X₂GXP)-n₂ (SEQ ID NO:8), wherein X, X₁, X₂ are any amino acid, wherein X, X₁, and X₂ can be the same or different amino acid, wherein n₁ and n₂ are equal to or greater than one; and
isolating the polypeptide.

13. The method of claim 12, wherein the polypeptide has the sequence selected from at least one of [(X₁X₂GXP)(X₃X₄GXP)]ₙ₁, [(X₁X₂GXP)ₙ₁(X₃X₄GXP)ₙ₂], or [(X₁X₂GXP)ₙ₁(X₃X₄GXP)ₙ₂(X₁X₂GXP)ₙ₁], wherein X, X₁, X₂, X₃, and X₄ are any amino acid, and n₁ and n₂ are greater than or equal to one; wherein X₁ and X₂ can be the same or different from each other, and X₃ and X₄ can be the same or different from each other; or wherein at least one of X₁ or X₂ is different from X₃ or X₄, or wherein X is valine, or X₁=G, X₂=Y and A (in 1:4 ratio) and X=V.

14. The method of claim 12, further comprising attaching to, or forming a fusion protein with, the polypeptide and at least a portion of an extracellular matrix component selected from at least one of: glycosaminoglycans (GAGs), proteoglycans, and/or proteins such as but not limited to laminin, fibronectin, vitronectin, collagen, elastin, fibrillin, fibulin, tenascin, perlecan, versican, aggrecan, neurocan, brevican, keratan, hyaluronic acid, heparan, or chondroitin, and wherein the fusion protein can be at an amino, a carboxy, or both the amino and carboxy ends of the polypeptide;
attaching to, or forming a fusion protein with, the polypeptide and at least a portion of a growth factor or cytokine selected from at least one of: leukemia inhibitory factor, insulin, insulin like growth factors, epidermal growth factor, fibroblast growth factors including basic fibroblast growth factor, vascular endothelial growth factor, transforming growth factor-β, platelet-derived growth factor, neurotrophic factors, interleukin-2, stem cell factor, Fms-like tyrosine kinase 3/fetal liver kinase-2, granulocyte-macrophage colony-stimulating factor, interleukin 1 alpha, or granulocyte colony-stimulating factor, and wherein the fusion protein can be at an amino, a carboxy, or both the amino and carboxy ends of the polypeptide;
attaching to, or forming a fusion protein with, the polypeptide and at least a portion of a growth factor or cytokine and an extracellular matrix component, wherein the fusion proteins can be at an amino, a carboxy, or both the amino and carboxy ends of the polypeptide; or
the polypeptide is provided in solution, attached to a substrate, or both.

15. The method of claim 12, wherein the polypeptide comprises at least one of: (1) a laminin domain comprising one or more VGKKKKKKKKG motifs (SEQ ID NO: 3); (2) one or more YIGSRVGKKKKKKKKG motifs (SEQ ID NO: 6); (3) one or more RNAIAEIIKDI motifs (SEQ ID NO: 2); (4) an elastin domain comprising one or more [(GAGVP)₂(GYGVP)(GAGVP)₂]₁₂ motifs (SEQ ID NO: 4); (5) [(GAGVP)₂(GYGVP)(GAGVP)₂]₂₄ motifs(SEQ ID NO: 5); (6) SEQ ID NOS: 1 and 4, (7) SEQ ID NOS: 1 and 5; (8) SEQ ID NOS: 3 and 4; (9) SEQ ID NOS: 3 and 5; (10) SEQ ID NOS: 2 and 4, or (6) SEQ ID NOS: 2 and 5; (11) any combination of SEQ ID NOS: 1, 2, 3, 4, or 5, wherein the polypeptide has the sequence selected from at least one of [(X₁X₂GXP)(X₃X₄GXP)]ₙ₁, [(X₁X₂GXP)ₙ₁(X₃X₄GXP)ₙ₂]; (12) [(X₁X₂GXP)ₙ₁(X₃X₄GXP)ₙ₂(X₁X₂GXP)ₙ₁], wherein X₁, X₂, X₃, X₄, and X are any amino acid except proline; (13) [(X₁X₂GXP)(X₃X₄GXP)]ₙ₁, [(X₁X₂GXP)ₙ₁(X₃X₄GXP)ₙ₂]; or (14) [(X₁X₂GXP)ₙ₁(X₃X₄GXP)ₙ₂(X₁X₂GXP)ₙ₁], wherein X₁, X₂, X₃, X₄ is any amino acid and X is an aliphatic amino acid; or the polypeptide is dissolved at a temperature below Tₜ before use; or the polypeptide is a recycled polypeptide prepared by: cycling the temperature of the polypeptide above and below Tₜ such that the polypeptide is at least one of (i) precipitated, (ii) washed, (iii) redissolved, and optionally steps (i) to (iii) can be repeated to remove impurities; or
further comprising the step of forming a 3D cell culture system, wherein the polypeptide creates a 3D scaffold for cell growth.

16. A method of making cardiomyocytes comprising:
seeding stem cells and incubating in a media that comprise a polypeptide that comprises one or more repeats of a sequence n₁-(X₁X₂GXP)-n₂ (SEQ ID NO:8), wherein X, X₁, X₂ are any amino acid, wherein X₁, X₂ and X can be the same or different amino acid, wherein n₁ and n₂ are equal to or greater than one;
culturing the stem cells without an anti-differentiation factor;
changing the media to cardiac differentiation media; and
isolating beating cardiomyocytes.

17. The method of claim 16, further comprising attaching to, or forming a fusion protein with, the polypeptide and at least a portion of an extracellular matrix component selected from at least one of: glycosaminoglycans (GAGs), proteoglycans, and/or proteins such as but not limited to laminin, fibronectin, vitronectin, collagen, elastin, fibrillin, fibulin, tenascin, perlecan, versican, aggrecan, neurocan, brevican, keratan, hyaluronic acid, heparan, or chondroitin, and wherein the fusion protein can be at an amino, a carboxy, or both the amino and carboxy ends of the polypeptide;
attaching to, or forming a fusion protein with, the polypeptide and at least a portion of a growth factor or cytokine selected from at least one of: leukemia inhibitory factor, insulin, insulin like growth factors, epidermal growth factor, fibroblast growth factors including basic fibroblast growth factor, vascular endothelial growth factor, transforming growth factor-β, platelet-derived growth factor, neurotrophic factors, interleukin-2, stem cell factor, Fms-like tyrosine kinase 3/fetal liver kinase-2, granulocyte-macrophage colony-stimulating factor, interleukin 1 alpha, or granulocyte colony-stimulating factor, and wherein the fusion protein can be at an amino, a carboxy, or both the amino and carboxy ends of the polypeptide;
attaching to, or forming a fusion protein with, the polypeptide and at least a portion of a growth factor or cytokine and an extracellular matrix component, wherein the fusion proteins can be at an amino, a carboxy, or both the amino and carboxy ends of the polypeptide; or
the polypeptide is provided in solution, attached to a substrate, or both.

18. The method of claim 16, wherein the polypeptide comprises at least one of: (1) a laminin domain comprising one or more VGKKKKKKKKG motifs (SEQ ID NO: 3); (2) one or more YIGSRVGKKKKKKKKG motifs (SEQ ID NO: 6); (3) one or more RNAIAEIIKDI motifs (SEQ ID NO: 2); (4) an elastin domain comprising one or more [(GAGVP)₂(GYGVP)(GAGVP)₂]₁₂ motifs (SEQ ID NO: 4); (5) [(GAGVP)₂(GYGVP)(GAGVP)₂]₂₄ motifs(SEQ ID NO: 5); (6) SEQ ID NOS: 1 and 4, (7) SEQ ID NOS: 1 and 5; (8) SEQ ID NOS: 3 and 4; (9) SEQ ID NOS: 3 and 5; (10) SEQ ID NOS: 2 and 4, or (6) SEQ ID NOS: 2 and 5; (11) any combination of SEQ ID NOS: 1, 2, 3, 4, or 5, wherein the polypeptide has the sequence selected from at least one of [(X₁X₂GXP)(X₃X₄GXP)]ₙ₁, [(X₁X₂GXP)ₙ₁(X₃X₄GXP)ₙ₂]; (12) [(X₁X₂GXP)ₙ₁(X₃X₄GXP)ₙ₂(X₁X₂GXP)ₙ₁], wherein X₁, X₂, X₃, X₄, and X are any amino acid except proline; (13) [(X₁X₂GXP)(X₃X₄GXP)]ₙ₁, [(X₁X₂GXP)ₙ₁(X₃X₄GXP)ₙ₂]; or (14) [(X₁X₂GXP)ₙ₁(X₃X₄GXP)ₙ₂(X₁X₂GXP)ₙ₁], wherein X₁, X₂, X₃, X₄ is any amino acid and X is an aliphatic amino acid.

19. The method of claim 16, wherein at least one of: the cardiac differentiation media does not include differentiation factors; the polypeptide is provided in a media at the same time as cells to be grown in the media or on a substrate; the cells for growth in a 3D culture system are primary cells, cell clones, cell lines, immortal cells, totipotent cells, multipotent cells, pluripotent cells, unipotent cells, stem cells, differentiated cells, or terminally differentiated cells; the cells are human cells; a substrate is a cell culture plate that comprises 1, 2, 4, 6, 8, 12, 16, 24, 32, 36, 48, 96, 192, or 384-well plates; or the cardiac differentiation media comprises at least one of: RA (retinoic acid); AA (Ascorbic acid); FGF8 (Fibroblast growth factor 8); SHH (Sonic hedgehog); bFGF (basic Fibroblast growth factor); BDNF (Brain-derived neurotrophic factor); GDNF (Glial cell-derived neurotrophic factor; CHIR99021 (Glycogen synthase kinase 3(GSK-3) Inhibitor); or cAMP (Cyclic adenosine monophosphate).

20. A beating cardiomyocyte made by a method comprising:
seeding stem cells in a media comprising a polypeptide that comprises one or more repeats of a sequence n₁-(X₁X₂GXP)-n₂ (SEQ ID NO:8), wherein X, X₁, X₂ are any amino acid, wherein X₁, X₂ and X can be the same or different amino acid, wherein n₁ and n₂ are equal to or greater than one;
culturing the stem cells without an anti-differentiation factor;
changing the media to cardiac differentiation media; and
isolating beating cardiomyocytes.

21. The method of claim 26, further comprising making a 3D cell culture comprising:
seeding cells and incubating in a media that comprises a polypeptide that comprises one or more repeats of a sequence n₁-(X₁X₂GXP)-n₂ (SEQ ID NO:8), wherein X, X₁, X₂ are any amino acid, wherein X₁, X₂ and X can be the same or different amino acid, wherein n₁ and n₂ are equal to or greater than one;
culturing cells;
changing the media; and
isolating the cells.

22. The method of claim 21, wherein the cells are stem cells for growth in the 3D system are differentiated into osteoblasts, osteoclasts, chondrocytes, adipocytes, fibroblasts, muscle cells, endothelial cells, epithelial cells, hematopoietic cells, sensory cells, endocrine and exocrine glandular cells, glia cells, neuronal cells, oligodendrocytes, blood cells, intestinal cells, cardiac cells, lung cells, liver cells, kidney cells, or pancreatic cells;
the cells for growth in a 3D system are primary cells, cell clones, cell lines, immortal cells, cancer cells, totipotent cells, multipotent cells, pluripotent cells, unipotent cells, stem cells, differentiated cells, or terminally differentiated cells;
the cells are human cells; or
the cells are bacterial cells, fungal cells, mammalian cells, insect cells, or plant cells.

23. The method of claim 21, wherein the polypeptide comprising a sequence (X₁X₂GVP)ₙ as a building block, where X₁ and X₂ are any amino acids except proline, and wherein X₁ and X₂ can be the same or different amino acids and wherein n is equal to or greater than one, wherein the polypeptide promotes cell growth in three dimensions;
attaching to, or forming a fusion protein with, the polypeptide and at least a portion of an extracellular matrix component selected from at least one of: glycosaminoglycans (GAGs), proteoglycans, and/or proteins such as but not limited to laminin, fibronectin, vitronectin, collagen, elastin, fibrillin, fibulin, tenascin, perlecan, versican, aggrecan, neurocan, brevican, keratan, hyaluronic acid, heparan, or chondroitin, and wherein the fusion protein can be at an amino, a carboxy, or both the amino and carboxy ends of the polypeptide;
attaching to, or forming a fusion protein with, the polypeptide and at least a portion of a growth factor or cytokine selected from at least one of: leukemia inhibitory factor, insulin, insulin like growth factors, epidermal growth factor, fibroblast growth factors including basic fibroblast growth factor, vascular endothelial growth factor, transforming growth factor-β, platelet-derived growth factor, neurotrophic factors, interleukin-2, stem cell factor, Fms-like tyrosine kinase 3/fetal liver kinase-2, granulocyte-macrophage colony-stimulating factor, interleukin 1 alpha, or granulocyte colony-stimulating factor, and wherein the fusion protein can be at an amino, a carboxy, or both the amino and carboxy ends of the polypeptide;
attaching to, or forming a fusion protein with, the polypeptide and at least a portion of a growth factor/cytokine and an extracellular matrix component, wherein the fusion proteins can be at an amino, a carboxy, or both the amino and carboxy ends of the polypeptide.; or
wherein the one or more growth factors are selected from at least one of: RA (retinoic acid); BMP4 (Bone morphogenetic protein; Activin A; bFGF (basic Fibroblast growth factor); VEGF (Vascular endothelial growth factor); AA (Ascorbic acid); CHIR99021 (Glycogen synthase kinase 3(GSK-3) Inhibitor); or DKK1 (Dickkopf-related protein 1).

24. The method of claim 21, wherein a 3D cell culture system comprises:
a substrate; and
a polypeptide that comprises one or more repeats of a sequence n₁-(X₁X₂GXP)-n₂ (SEQ ID NO:8), wherein X, X₁, X₂ are any amino acid, wherein X₁, X₂ and X can be the same or different amino acid, wherein n₁ and n₂ are equal to or greater than one, wherein the polypeptide promotes cell growth in three dimensions.

25. The system of claim 24, wherein at least one of: the polypeptide comprises a sequence (X₁X₂GVP)ₙ as a building block, where X₁ and X₂ are any amino acids except proline, and wherein X₁ and X₂ can be the same or different amino acids and wherein n is equal to or greater than 1;
the polypeptide is mixed in a media or attached or adhered to the substrate;
the polypeptide promotes totipotency, pluripotency, multipotency, or unipotency;
the substrate is a gelatin-coated dish;
the polypeptide is provided in a media at the same time as cells to be grown in the system;
the one or more cells for growth in the 3D system are primary cells, cell clones, cell lines, immortal cells, cancer cells, totipotent cells, multipotent cells, pluripotent cells, unipotent cells, stem cells, differentiated cells, or terminally differentiated cells;
the cells grown in three dimensions are human cells;
the substrate is a cell culture plate that comprises 1, 2, 4, 6, 8, 12, 16, 24, 32, 36, 48, 96, 192, or 384-well plates;
the substrate is charged with a positive or negative charge;
the substrate is selected from at least one of polystyrene, polypropylene, polymethyl methacrylate, polyvinyl chloride, polymethyl pentene, polyethylene, polycarbonate, polysulfone, polystyrene, fluoropolymers, polyamides, or silicones;
further comprises a thixotropic agent; or
wherein a single building block sequence is used, that is the sequence of polypeptide is (X₁X₂GVP)ₙ, and n is greater than or equal to zero.

26. The system of claim 25, wherein the more than one different type of building block is joined in any order to construct the polypeptide comprising [(X₁X₂GVP)(X₃X₄GVP)]ₙ₁, [(X₁X₂GVP)ₙ₁(X₃X₄GVP)ₙ₂], or [(X₁X₂GVP)ₙ₁(X₃X₄GVP)ₙ₂(X₁X₂GVP)ₙ₁], wherein X₁, X₂, X₃, and X₄ are any amino acid except proline, and n₁ and n₂ are greater than or equal to one, or X₁=G, X₂=Y and A (in 1:4 ratio) and X=V.

27. The system of claim 24, wherein X₁ and X₂ can be the same or different from each other, and X₃ and X₄ can be the same or different from each other, however, at least one of X₁ or X₂ is different from X₃ or X₄ to obtain different building blocks.

28. The system of claim 24, wherein the polypeptide is attached to or a fusion protein with an extracellular matrix component selected from at least one of: glycosaminoglycans (GAGs), proteoglycans, or proteins;
attaching to, or forming a fusion protein with, the polypeptide and at least a portion of an extracellular matrix component selected from at least one of: glycosaminoglycans (GAGs), proteoglycans, and/or proteins such as but not limited to laminin, fibronectin, vitronectin, collagen, elastin, fibrillin, fibulin, tenascin, perlecan, versican, aggrecan, neurocan, brevican, keratan, hyaluronic acid, heparan, or chondroitin, and wherein the fusion protein can be at an amino, a carboxy, or both the amino and carboxy ends of the polypeptide;
attaching to, or forming a fusion protein with, the polypeptide and at least a portion of a growth factor or cytokine selected from at least one of: leukemia inhibitory factor, insulin, insulin like growth factors, epidermal growth factor, fibroblast growth factors including basic fibroblast growth factor, vascular endothelial growth factor, transforming growth factor-β, platelet-derived growth factor, neurotrophic factors, interleukin-2, stem cell factor, Fms-like tyrosine kinase 3/fetal liver kinase-2, granulocyte-macrophage colony-stimulating factor, interleukin 1 alpha, or granulocyte colony-stimulating factor, and wherein the fusion protein can be at an amino, a carboxy, or both the amino and carboxy ends of the polypeptide; or
attaching to, or forming a fusion protein with, the polypeptide and at least a portion of a growth factor/cytokine and an extracellular matrix component, wherein the fusion proteins can be at an amino, a carboxy, or both the amino and carboxy ends of the polypeptide.
